(19) United States
(12) Patent Application Publication
(10) Pub. No.: US 2024/0084309 A1
(43) Pub. Date: Mar. 14, 2024

(54) COMPOSITIONS AND METHODS FOR INHIBITING PLP1 EXPRESSION

(71) Applicant: Dicerna Pharmaceuticals, Inc., Lexington, MA (US)

(72) Inventors: Bob Dale BROWN, Littleton, MA (US); Maire JUNG, Lincoln, MA (US); Armen CHANGELIAN, Cambridge, MA (US); Chunyang ZHANG, Burlington, MA (US)

(21) Appl. No.: 18/019,531

(22) PCT Filed: Aug. 4, 2021
(86) PCT No.: PCT/US2021/044541

§ 371 Date: Feb. 3, 2023

Related U.S. Application Data
(60) Provisional application No. 63/151,445, filed on Feb. 19, 2021.
(60) Provisional application No. 63/061,040, filed on Aug. 4, 2020.

(51) Int. Cl. C12N 15/113; A61K 47/26; A61K 47/54; A61P 25/00; C12Q 1/6883

(52) U.S. Cl. CPC C12N 15/1138; A61K 47/26; A61K 47/555; A61P 25/00; C12Q 1/6883

(57) ABSTRACT

Oligonucleotides are provided herein that inhibit PLP1 expression. Also provided are compositions including the same and uses thereof, particularly uses relating to treating diseases, disorders and/or conditions associated with PLP1 expression.

### CROSS-RELATED APPLICATIONS

This application claims the benefit of U.S. Provisional Patent Application Ser. No. 63,061,040 filed Aug. 4, 2020, and U.S. Provisional Patent Application Ser. No. 63/151,445, filed Feb. 19, 2021. The entire contents of which is incorporated herein by this reference.

### BACKGROUND

Myelin proteolipid protein (PLP) is the most abundant protein found in myelin from the central nervous system (CNS). The tetraspan protein plays a major role in the structure and function of myelin and is important for communication between oligodendrocytes and axons (Gruenenfelder et al., J ANAT, 219: 33-43 (2011)). The protein serves a neuroprotective role; it is required for proper sequestration of proteins into the myelin compartment, allegedly important for axo-glial metabolism and long-term support of axons (Werner et al., J NEUROSCI, 27: 7717-30 (2007)).

Expression of the PLP1 gene is regulated in a spatio-temporal manner (Wight and Dobretsova, CELL MOL LIFE SCI, 61: 810-21 (2004)). High levels are produced in oligodendrocytes concurrent with the active myelination period of development. Its expression must be tightly regulated as evidenced by X-linked genetic diseases associated with the expression of PLP1.

In humans, both segmental deletion (Raskind et al., AM J HUM GENET, 49: 1355-60 (1991); Inoue et al., AM J HUM GENET, 71: 838-853 (2002)) and duplication (Sistermans et al., NEUROLOGY, 50: 1749-54 (1998); Inoue et al., ANN NEUROL, 45: 624-32 (1999); Mimault et al., AM J HUM GENET, 65: 360-69 (1999); Hodes et al., AM J HUM GENET, 67: 14-22 (2000)) or higher copy number (Wolf et al., BRAIN 128: 743-51 (2005)) of the chromosomal region containing the PLP1 gene can lead to the dysmyelinating disorder Pelizaeus-Merzbacher disease (PMD) or the related allelic disorder, spastic paraplegia type 2 (SPG2).

Strategies for targeting the PLP1 gene to prevent such diseases are needed.

### SUMMARY OF DISCLOSURE

The disclosure is based, at least in part, on the discovery that small interfering RNA (siRNA), including synthetic RNAi oligonucleotides as described herein, are useful to silence mRNA encoding proteins associated with neurological disease or disorder in brain tissue, such as mRNA expressed in neurons and glial cells, including oligodendrocytes, microglia and astrocytes. It was surprisingly discovered that such RNAi oligonucleotides effectively silence mRNA expressed in glial cells such as oligodendrocytes in the absence of a cell- or tissue-specific targeting ligand or delivery vehicle such as a viral vector, liposome or lipid nanoparticle. It was also discovered that such RNAi oligonucleotides, when formulated with a pharmaceutically acceptable carrier, provide compositions for direct administration to the cerebral spinal fluid (CSF) of a subject, such as by intrathecal, intracerebroventricular, or intracisternal magna administration. Unexpectedly, such RNAi oligonucleotides demonstrated durable knockdown of expression of a target mRNA (PLP1) in various brain regions up to 84 days following a single or repeated administration of the RNAi oligonucleotide into the CFS of non-human primates.

The disclosure is based in part on the discovery that double-stranded oligonucleotides (e.g., RNAi oligonucleotides) reduce PLP1 expression in the central nervous system (CNS). Accordingly, target sequences within PLP1 mRNA were identified and RNAi oligonucleotides that bind to these target sequences and inhibit PLP1 mRNA expression were generated. As demonstrated herein, the RNAi oligonucleotides inhibited murine Plp1 expression, and/or monkey and human PLP1 expression in different regions of the CNS. Without being bound by theory, the RNAi oligonucleotides described herein are useful for treating a disease, disorder or condition associated with PLP1 expression (e.g., Pelizaeus-Merzbacher disease (PMD) and/or spastic paraplegia type 2 (SPG2)). In some embodiments, the RNAi oligonucleotides described herein are useful for treating a disease, disorder or condition associated with aberrant PLP1 expression (e.g, PLP1 overexpression resulting from PLP1 gene duplication or expression of a deleterious PLP1 mutant allele). In some embodiments, the RNAi oligonucleotides described herein are useful for treating a disease, disorder or condition associated with mutations in the PLP1 gene.

As PLP1 duplication is a common mutation associated with PMD, a mouse model having duplication of the murine Plp1 gene was utilized to demonstrate the efficacy of the RNAi oligonucleotides described herein. Not only did an RNAi oligonucleotide targeting murine Plp1 reduce levels of both Plp1 mRNA and protein, but also reversed astrogliosis and dysmyelination induced in the duplication model. Notably, as PLP1 is necessary for normal brain function, it was demonstrated that the RNAi oligonucleotide did not completely ablate Plp1 expression, but reduced levels of Plp1 similar to those expressed in wild-type mice. Without wishing to be bound by theory, these results indicate the RNAi oligonucleotides described herein are not only useful for treating a disease, disorder or condition associated with PLP1 expression but is useful to maintain sufficient levels of PLP1 expression to support beneficial brain function and avoid unwanted side effects.

It has also been demonstrated that RNAi oligonucleotides targeting PLP1 reduce expression of glial fibrillary acidic protein (GFAP), a marker of astrogliosis. Astrogliosis occurs when astrocytes respond to damage and disease in the CNS. As described herein, increases in GFAP expression occur in a mouse model having duplication of Plp1, indicating damage in the CNS. As demonstrated herein, RNAi oligonucleotides targeting PLP1 not only reduced PLP1 expression in this mouse model, but also reduced GFAP. Without wishing to be bound by theory, monitoring expression level of GFAP in subjects who have received or are receiving treatment with an RNAi oligonucleotide of the disclosure is useful for monitoring treatment outcomes, monitoring progression of disease, condition or disorder associated with PLP1 expression and/or for determining responsiveness to treatment with an RNAi oligonucleotide of the disclosure.

Accordingly, in some aspects, the present disclosure provides an RNAi oligonucleotide for reducing PLP1 expression, the oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

In some aspects, the disclosure provides an RNAi oligonucleotide for reducing PLP1 expression, the oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 212-231, and wherein the region of complementarity is at least 15 contiguous nucleotides in length.

In any of the foregoing or related aspects, the sense strand is 15 to 50 nucleotides in length. In some aspects, the sense strand is 18 to 36 nucleotides in length.

In any of the foregoing or related aspects, the antisense strand is 15 to 30 nucleotides in length.

In any of the foregoing or related aspects, the anti sense strand is 22 nucleotides in length and wherein antisense strand and the sense strand form a duplex region of at least 19 nucleotides in length, optionally at least 20 nucleotides in length.

In any of the foregoing or related aspects, the region of complementarity is at least 19 contiguous nucleotides in length, optionally at least 20 nucleotides in length.

In any of the foregoing or related aspects, the 3′ end of the sense strand comprises a stem-loop set forth as S1-L-S2, wherein S1 is complementary to S2, and wherein L forms a loop between S1 and S2 of 3-5 nucleotides in length.

In some aspects, the disclosure provides an RNAi oligonucleotide for reducing PLP1 expression, the oligonucleotide comprising a sense strand of 15 to 50 nucleotides in length and an antisense strand, wherein the sense strand and the antisense strand form a duplex region, wherein the wherein the antisense strand comprises a region of complementarity to a PLP1 mRNA target sequence of any one of SEQ ID NOs: 171-188, and wherein the region of complementarity is at least 15 contiguous nucleotides in length.

In other aspects, the disclosure provides an RNAi oligonucleotide for reducing PLP1 expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

In yet other aspects, the disclosure provides an RNAi oligonucleotide for reducing PLP1 expression, the oligonucleotide comprising a sense strand of 15 to 50 nucleotides in length and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In further aspects, the disclosure provides an RNAi oligonucleotide for reducing PLP1 expression, the oligonucleotide comprising a sense strand of 18 to 36 nucleotides in length and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In other aspects, the disclosure provides an RNAi oligonucleotide for reducing PLP1 expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In some aspects, the disclosure provides an RNAi oligonucleotide for reducing PLP1 expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between 51 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In other aspects, the disclosure provides an RNAi oligonucleotide for reducing PLP1 expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In yet other aspects, the disclosure provides an RNAi oligonucleotide for reducing PLP1 expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

A double stranded RNAi oligonucleotide for reducing PLP1 expression, the oligonucleotide comprising:
(i) an antisense strand of 19-30 nucleotides in length, wherein the antisense strand comprises a nucleotide sequence comprising a region of complementarity to a PLP1 mRNA target sequence, wherein the region of complementarity is selected from SEQ ID NOs: 235-254, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand. In some aspects, the sense strand comprises a nucleotide sequence selected from SEQ ID Nos: 212-231.

In any of the foregoing or related aspects, the target sequence comprises any one of SEQ ID Nos: 212-231.

In any of the foregoing or related aspects, the region of complementarity differs by no more than 3 nucleotides in length to the PLP1 mRNA target sequence. In other aspects, the region of complementarity is fully complementary to the PLP1 mRNA target sequence.

In any of the foregoing or related aspects, L is a triloop or a tetraloop. In some aspects, L is a tetraloop. In some aspects, the tetraloop comprises the sequence 5′-GAAA-3′. In some aspects, one or more of the nucleotides of L comprise a 2′-O-methyl modification. In some aspects, each nucleotide of L comprises a 2′-O-methyl modification.

In any of the foregoing or related aspects, the S1 and S2 are 1-10 nucleotides in length and have the same length. In some aspects, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some aspects, S1 and S2 are 6 nucleotides in length. In some aspects, the stem-loop comprises the sequence 5′-GCAGCCGAAAGGCUGC-3′ (SEQ ID NO: 190).

In any of the foregoing or related aspects, the antisense strand comprises a 3′ overhang sequence of one or more nucleotides in length. In some aspects, the 3′ overhang sequence is 2 nucleotides in length, optionally wherein the 3′ overhang sequence is GG.

In any of the foregoing or related aspects, the oligonucleotide comprises at least one modified nucleotide. In some aspects, the modified nucleotide comprises a 2′-modification. In some aspects, the 2′-modification is a modification selected from T-aminoethyl, 2′-fluoro, 2′-O-methyl, 2′-O-methoxyethyl, and 2′-deoxy-2′-fluoro-β-d-arabinonucleic acid. In some aspects, all nucleotides comprising the oligonucleotide are modified, optionally wherein the modification is a 2′-modification selected from 2′-fluoro and 2′-O-methyl.

In any of the foregoing or related aspects, about 10-15%, 10%, 11%, 12%, 13%, 14% or 15% of the nucleotides of the sense strand comprise a 2′-fluoro modification. In some aspects, about 25-35%, 25%, 26%, 27%, 28%, 29%, 30%, 31%, 32%, 33%, 34% or 35% of the nucleotides of the antisense strand comprise a 2′-fluoro modification. In some aspects, about 15-25%, 15%, 16%, 17%, 18%, 19%, 20%, 21%, 22%, 23%, 24%, or 25% of the nucleotides of the oligonucleotide comprise a 2′-fluoro modification.

In any of the foregoing or related aspects, the sense strand comprises 36 nucleotides with positions numbered 1-36 from 5′ to 3′, wherein positions 8-11 comprise a 2′-fluoro modification. In some aspects, the antisense strand comprises 22 nucleotides with positions numbered 1-22 from 5′ to 3′, and wherein positions 2, 3, 4, 5, 7, 10 and 14 comprise a 2′-fluoro modification. In some aspects, the remaining nucleotides of the oligonucleotide comprise a 2′-O-methyl modification.

In any of the foregoing or related aspects, the oligonucleotide comprises at least one modified internucleotide linkage. In some aspects, the at least one modified internucleotide linkage is a phosphorothioate linkage.

In any of the foregoing or related aspects, the 4′-carbon of the sugar of the 5′-nucleotide of the antisense strand comprises a phosphate analog. In some aspects, the phosphate analog is oxymethylphosphonate, vinylphosphonate or malonylphosphonate, optionally wherein the phosphate analog is a 4′-phosphate analog comprising 5′-methoxyphosphonate-4′-oxy. In some aspects, the phosphate analog is a 4′-oxymethylphosphonate.

In any of the foregoing or related aspects, at least one nucleotide of the oligonucleotide is conjugated to one or more targeting ligands. In some aspects, each targeting ligand comprises a carbohydrate, amino sugar, cholesterol, polypeptide or lipid. In some aspects, each targeting ligand comprises a N-acetylgalactosamine (GalNAc) moiety. In some aspects, the GalNac moiety is a monovalent GalNAc moiety, a bivalent GalNAc moiety, a trivalent GalNAc moiety or a tetravalent GalNAc moiety. In some aspects, up to 4 nucleotides of L of the stem-loop are each conjugated to a monovalent GalNAc moiety.

In some aspects, the oligonucleotide does not comprise a targeting ligand.

In any of the foregoing or related aspects, the sense strand comprises a nucleotide sequence of any one of SEQ ID NOs: 76, 78, 80, 82, 84, 86, 88, 90, 92, 94, 96, 98, 100, 102, 104, 106, 108, and 110.

In any of the foregoing or related aspects, the antisense strand comprises a nucleotide sequence of any one of SEQ ID NOs: 77, 79, 81, 83, 85, 87, 89, 91, 93, 95, 97, 99, 101, 103, 105, 107, 109, and 111.

In any of the foregoing or related aspects, the sense strand and antisense strands comprise nucleotide sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

In some aspects, the sense strand comprises a nucleotide sequence as set forth in SEQ ID NO: 76, wherein the antisense strand comprises a nucleotide sequence as set forth in SEQ ID NO: 77. In other aspects, the sense strand comprises a nucleotide sequence as set forth in SEQ ID NO: 78, wherein the antisense strand comprises a nucleotide sequence as set forth in SEQ ID NO: 79. In other aspects, the sense strand comprises a nucleotide sequence as set forth in SEQ ID NO: 80, wherein the antisense strand comprises a nucleotide sequence as set forth in SEQ ID NO: 81. In some aspects, the sense strand comprises a nucleotide sequence as set forth in SEQ ID NO: 82, wherein the antisense strand comprises a nucleotide sequence as set forth in SEQ ID NO: 83. In other aspects, the sense strand comprises a nucleotide sequence as set forth in SEQ ID NO: 84, wherein the antisense strand comprises a nucleotide sequence as set forth in SEQ ID NO: 85. In yet other aspects, the sense strand comprises a nucleotide sequence as set forth in SEQ ID NO: 86, wherein the antisense strand comprises a nucleotide sequence as set forth in SEQ ID NO: 87. In some aspects, the sense strand comprises a nucleotide sequence as set forth in SEQ ID NO: 88, wherein the antisense strand comprises a nucleotide sequence as set forth in SEQ ID NO: 89. In other aspects, the sense strand comprises a nucleotide sequence as set forth in SEQ ID NO: 90, wherein the antisense strand comprises a nucleotide sequence as set forth in SEQ ID NO: 91. In yet other aspects, the sense strand comprises a nucleotide sequence as set forth in SEQ ID NO: 92, wherein the antisense strand comprises a nucleotide sequence as set forth in SEQ ID NO: 93. In some aspects, the sense strand comprises a nucleotide sequence as set forth in SEQ ID NO: 94, wherein the antisense strand comprises a nucleotide sequence as set forth in SEQ ID NO: 95. In other aspects, the sense strand comprises a nucleotide sequence as set forth in SEQ ID NO: 96, wherein the antisense strand comprises a nucleotide sequence as set forth in SEQ ID NO: 97. In yet other aspects, the sense strand comprises a nucleotide sequence as set forth in SEQ ID NO: 98, wherein the antisense strand comprises a nucleotide sequence as set forth in SEQ ID NO: 99. In some aspects, the sense strand comprises a nucleotide sequence as set forth in SEQ ID NO: 100, wherein the antisense strand comprises a nucleotide sequence as set forth in SEQ ID NO: 101. In other aspects, the sense strand comprises a nucleotide sequence as set forth in SEQ ID NO: 102, wherein the antisense strand comprises a nucleotide sequence as set forth in SEQ ID NO: 103. In yet other aspects, the sense strand comprises a nucleotide sequence as set forth in SEQ ID NO: 104, wherein the antisense strand comprises a nucleotide sequence as set forth in SEQ ID NO: 105. In some aspects, the sense strand comprises a nucleotide sequence as set forth in SEQ ID NO: 106, wherein the antisense strand comprises a nucleotide sequence as set forth in SEQ ID NO: 107. In other aspects, the sense strand comprises a nucleotide sequence as set forth in SEQ ID NO: 108, wherein the antisense strand comprises a nucleotide sequence as set forth in SEQ ID NO: 109. In yet other aspects, the sense strand comprises a nucleotide sequence as set forth in SEQ ID NO: 110, wherein the antisense strand comprises a nucleotide sequence as set forth in SEQ ID NO: 111.

In some aspects, the disclosure provides an RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the antisense strand comprises a region of complementarity to a PLP1 mRNA target sequence of any one of SEQ ID NOs: 171-188, and wherein the region of complementarity is at least 15 contiguous nucleotides in length.

In further aspects, the disclosure provides an RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

In other aspects, the disclosure provides an RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

In some aspects, the disclosure provides an RNAi oligonucleotide for reducing PLP1 expression, the oligonucleotide comprising a sense strand and an antisense strand, wherein the sense strand and the antisense strand form a duplex region, wherein all nucleotides comprising the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

In any of the foregoing or related aspects, the sense strand comprises of any one of SEQ ID NOs: 112, 114, 116, 118, 120, 122, 124, 126, 128, 130, 132, 134, 136, 138, 140, 142, 144, 146, and 191.

In any of the foregoing or related aspects, the antisense strand comprises of any one of SEQ ID NOs: 113, 115, 117, 119, 121, 123, 125, 127, 129, 131, 133, 135, 137, 139, 141, 143, 145, 147 and 192. In any of the foregoing or related aspects, the antisense strand comprises of any one of SEQ ID NOs: 113, 115, 117, 119, 121, 123, 125, 127, 129, 131, 133, 135, 137, 139, 141, 143, 145, 147 and 207.

In any of the foregoing or related aspects, the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 192, respectively.

In any of the foregoing or related aspects, the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 207, respectively.

In some aspects, the sense strand comprises SEQ ID NO: 112, and wherein the antisense strand comprises SEQ ID NO: 113. In other aspects, the sense strand comprises SEQ ID NO: 114, and wherein the antisense strand comprises SEQ ID NO: 115. In yet other aspects, the sense strand comprises SEQ ID NO: 116, and wherein the antisense strand comprises SEQ ID NO: 117. In some aspects, the sense strand comprises SEQ ID NO: 118, and wherein the antisense strand comprises SEQ ID NO: 119. In other aspects, the sense strand comprises SEQ ID NO: 120, and wherein the antisense strand comprises SEQ ID NO: 121. In yet other aspects, the sense strand comprises SEQ ID NO: 122, and wherein the antisense strand comprises SEQ ID NO: 123. In some aspects, the sense strand comprises SEQ ID NO: 124, and wherein the antisense strand comprises SEQ ID NO: 125. In other aspects, the sense strand comprises SEQ ID NO: 126, and wherein the antisense strand comprises SEQ ID NO: 127. In some aspects, the sense strand comprises SEQ ID NO: 128, and wherein the antisense strand comprises SEQ ID NO: 129. In other aspects the sense strand comprises SEQ ID NO: 130, and wherein the antisense strand comprises SEQ ID NO: 131. In yet other aspects, the sense strand comprises SEQ ID NO: 132, and wherein the antisense strand comprises SEQ ID NO: 133. In some aspects, the sense strand comprises SEQ ID NO: 134, and wherein the antisense strand comprises SEQ ID NO: 135. In other aspects, the sense strand comprises SEQ ID NO: 136, and wherein the antisense strand comprises SEQ ID NO: 137. In yet other aspects, the sense strand comprises SEQ ID NO: 138, and wherein the antisense strand comprises SEQ ID NO: 139. In some aspects, the sense strand comprises SEQ ID NO: 140, and wherein the antisense strand comprises SEQ ID NO: 141. In other aspects, the sense strand comprises SEQ ID NO: 142, and wherein the antisense strand comprises SEQ ID NO: 143. In some aspects, the sense strand comprises SEQ ID NO: 144, and wherein the antisense strand comprises SEQ ID NO: 145. In other aspects, the sense strand comprises SEQ ID NO: 146, and wherein the antisense strand comprises SEQ ID NO: 147. In other aspects, the sense strand comprises SEQ ID NO: 191, and wherein the antisense strand comprises SEQ ID NO: 192. In other aspects, the sense strand comprises SEQ ID NO: 191, and wherein the antisense strand comprises SEQ ID NO: 207.

In some aspects, the disclosure provides a method for treating a subject having a disease, disorder or condition associated with PLP1 expression, the method comprising administering to the subject a therapeutically effective amount of an RNAi oligonucleotide described herein, or pharmaceutical composition thereof, thereby treating the subject. In some aspects, the RNAi oligonucleotide is administered to the central nervous system. In some aspects, the RNAi oligonucleotide is administered to the cerebral spinal fluid. In some aspects, the RNAi oligonucleotide is administered intrathecally, intracerebroventricularly, or by intracisternal magna injection. In some aspects, a single dose of the RNAi oligonucleotide is administered. In other aspects, more than one dose of the RNAi oligonucleotide is administered.

In any of the foregoing or related aspects, PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks. In some aspects, PLP1 expression is reduced for about 1 month, 2 months, 3 months, 4 months, 5 months or 6 months. In some aspects, PLP1 expression is reduced for about 7 days, 14 days, 21 days, 28 days, 35 days, 42 days, 49 days, 56 days, 63 days, 70 days, 77 days, 84 days, or 91 days.

In some aspects, the disclosure provides a pharmaceutical composition comprising a RNAi oligonucleotide described herein, and a pharmaceutically acceptable carrier, delivery agent or excipient.

In other aspects, the disclosure provides a method of delivering an oligonucleotide to a subject, the method comprising administering a pharmaceutical composition described herein to the subject.

In another aspect, the disclosure provides a method for reducing PLP1 expression in a cell, a population of cells or a subject, the method comprising the step of:
i. contacting the cell or the population of cells with a RNAi oligonucleotide or pharmaceutical composition described herein; or
ii. administering to the subject a RNAi oligonucleotide or pharmaceutical composition described herein. In some aspects, reducing PLP1 expression comprises reducing an amount or level of PLP1 mRNA, an amount or level of PLP1 protein, or both. In some aspects, PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks. In some aspects, PLP1 expression is reduced for about 1 month, 2 months, 3 months, 4 months, 5 months or 6 months. In some aspects, PLP1 expression is reduced for about 7 days, 14 days, 21 days, 28 days, 35 days, 42 days, 49 days, 56 days, 63 days, 70 days, 77 days, 84 days, or 91 days. In some aspects, the subject has a disease, disorder or condition associated with PLP1 expression. In some aspects, the disease, disorder or condition associated with PLP1 expression is Pelizaeus-Merzbacher disease (PMD) or spastic paraplegia type 2 (SPG2). In some aspects, the RNAi oligonucleotide, or pharmaceutical composition, is administered in combination with a second composition or therapeutic agent.

In other aspects, the disclosure provides a method for treating a subject having a disease, disorder or condition associated with PLP1 expression, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

In another aspect, the disclosure provides a method for treating a subject having a disease, disorder or condition associated with PLP1 expression, the method comprising administering to the subject a therapeutically effective amount of an RNAi oligonucleotide comprising a sense strand and an antisense strand selected from a row set forth in Table 5, or pharmaceutical composition thereof, thereby treating the subject.

In other aspects, the disclosure provides a method for treating a subject having a disease, disorder or condition associated with PLP1 expression, the method comprising administering to the subject a therapeutically effective amount of an RNAi oligonucleotide comprising a sense strand and an antisense strand, wherein the sense strand and antisense strands comprise nucleotide sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

In other aspects, the disclosure provides a method for treating a subject having a disease, disorder or condition associated with PLP1 expression, the method comprising administering to the subject a therapeutically effective amount of an RNAi oligonucleotide comprising a sense strand and an antisense strand, wherein the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 192, respectively.

In other aspects, the disclosure provides a method for treating a subject having a disease, disorder or condition associated with PLP1 expression, the method comprising administering to the subject a therapeutically effective amount of an RNAi oligonucleotide comprising a sense strand and an antisense strand, wherein the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 207, respectively.

In some aspects, the sense strand comprises SEQ ID NO: 112, and wherein the antisense strand comprises SEQ ID NO: 113. In other aspects, the sense strand comprises SEQ ID NO: 114, and wherein the antisense strand comprises SEQ ID NO: 115, In yet other aspects, the sense strand comprises SEQ ID NO: 116, and wherein the antisense strand comprises SEQ ID NO: 117. In some aspects, the sense strand comprises SEQ ID NO: 118, and wherein the antisense strand comprises SEQ ID NO: 119. In other aspects, the sense strand comprises SEQ ID NO: 120, and wherein the antisense strand comprises SEQ ID NO: 121. In yet other aspects, the sense strand comprises SEQ ID NO: 122, and wherein the antisense strand comprises SEQ ID NO: 123. In some aspects, the sense strand comprises SEQ ID NO: 124, and wherein the antisense strand comprises SEQ ID NO: 125. In other aspects, the sense strand comprises SEQ ID NO: 126, and wherein the antisense strand comprises SEQ ID NO: 127. In some aspects, the sense strand comprises SEQ ID NO: 128, and wherein the antisense strand comprises SEQ ID NO: 129. In other aspects the sense strand comprises SEQ ID NO: 130, and wherein the antisense strand comprises SEQ ID NO: 131. In yet other aspects, the sense strand comprises SEQ ID NO: 132, and wherein the antisense strand comprises SEQ ID NO: 133. In some aspects, the sense strand comprises SEQ ID NO: 134, and wherein the antisense strand comprises SEQ ID NO: 135. In other aspects, the sense strand comprises SEQ ID NO: 136, and wherein the antisense strand comprises SEQ ID NO: 137. In yet other aspects, the sense strand comprises SEQ ID NO: 138, and wherein the antisense strand comprises SEQ ID NO: 139. In some aspects, the sense strand comprises SEQ ID NO: 140, and wherein the antisense strand comprises SEQ ID NO: 141. In other aspects, the sense strand comprises SEQ ID NO: 142, and wherein the antisense strand comprises SEQ ID NO: 143. In some aspects, the sense strand comprises SEQ ID NO: 144, and wherein the antisense strand comprises SEQ ID NO: 145. In other aspects, the sense strand comprises SEQ ID NO: 146, and wherein the antisense strand comprises SEQ ID NO: 147. In other aspects, the sense strand comprises SEQ ID NO: 191, and wherein the antisense strand comprises SEQ ID NO: 192. In other aspects, the sense strand comprises SEQ ID NO: 191, and wherein the antisense strand comprises SEQ ID NO: 207.

In some aspects, the disease, disorder or condition associated with PLP1 expression is Pelizaeus-Merzbacher disease (PMD) or spastic paraplegia type 2 (SPG2).

In any of the foregoing or related aspects, the RNAi oligonucleotide is administered to the central nervous system. In some aspects, the RNAi oligonucleotide is administered to the cerebral spinal fluid. In some aspects, the RNAi oligonucleotide is administered intrathecally, intracerebroventricularly, or by intracisternal magna injection. In some aspects, a single dose of the RNAi oligonucleotide is administered. In other aspects, more than one dose of the RNAi oligonucleotide is administered.

In any of the foregoing or related aspects, PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks. In some aspects, PLP1 expression is reduced for about 1 month, 2 months, 3 months, 4 months, 5 months or 6 months. In some aspects, PLP1 expression is reduced for about 7 days, 14 days, 21 days, 28 days, 35 days, 42 days, 49 days, 56 days, 63 days, 70 days, 77 days, 84 days, or 91 days.

In some aspects, the disclosure provides use of an RNAi oligonucleotide or pharmaceutical composition described herein, in the manufacture of a medicament for the treatment of a disease, disorder or condition associated with PLP1 expression, optionally for the treatment of Pelizaeus-Merzbacher disease (PMD) or spastic paraplegia type 2 (SPG2).

In some aspects, the disclosure provides use of an RNAi oligonucleotide or pharmaceutical composition described herein, for use, or adaptable for use, in the treatment of a disease, disorder or condition associated with PLP1 expression, optionally for the treatment of Pelizaeus-Merzbacher disease (PMD) or spastic paraplegia type 2 (SPG2).

In other aspects, the disclosure provides a kit comprising an RNAi oligonucleotide described herein, an optional pharmaceutically acceptable carrier, and a package insert comprising instructions for administration to a subject having a disease, disorder or condition associated with PLP1 expression.

In any of the foregoing or related aspects, the disease, disorder or condition associated with PLP1 expression is Pelizaeus-Merzbacher disease (PMD) or spastic paraplegia type 2 (SPG2).

In some aspects, the disclosure provides a composition comprising an RNAi oligonucleotide for reducing PLP1 expression and a pharmaceutically acceptable carrier, wherein the oligonucleotide comprises a sense strand and an antisense strand that form a duplex region, wherein the antisense strand comprises a region of complementarity to a PLP1 mRNA target sequence, wherein the region of complementarity is at least 15 contiguous nucleotides in length, and wherein the composition is formulated for administration to the cerebral spinal fluid (CSF) of a subject. In some aspects, the RNAi oligonucleotide is an RNAi oligonucleotide described herein. In some aspects, the composition is formulated for intrathecal, intracerebroventricular, or intracisternal magna administration. In some aspects, the oligonucleotide does not comprise a targeting ligand. In some aspects, the oligonucleotide is not formulated in a lipid, liposome or lipid nanoparticle delivery vehicle. In some aspects, the pharmaceutically acceptable carrier comprises phosphate buffered saline.

In other aspects, the disclosure provides a method for reducing expression of PLP1 in the central nervous system of a subject, comprising administering a composition comprising an RNAi oligonucleotide and a pharmaceutically acceptable carrier, wherein the RNAi oligonucleotide comprises a sense strand and an antisense strand that form a duplex region, wherein the antisense strand comprises a region of complementarity to a PLP1 mRNA, wherein the region of complementarity is at least 15 contiguous nucleotides in length, and wherein the composition is formulated for administration to the cerebral spinal fluid (CSF), thereby reducing PLP1 expression in the central nervous system. In some aspects, the RNAi oligonucleotide is an RNAi oligonucleotide described herein. In some aspects, a single dose or more than one dose of RNAi oligonucleotide is administered. In some aspects, PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks. In some aspects, PLP1 expression is reduced for about 1 month, 2 months, 3 months, 4 months, 5 months or 6 months. In some aspects, PLP1 expression is reduced for about 7 days, 14 days, 21 days, 28 days, 35 days, 42 days, 49 days, 56 days, 63 days, 70 days, 77 days, 84 days, or 91 days. In some aspects, PLP1 expression is reduced in at least one region of the brain. In some aspects, the at least one region of the brain is selected from: frontal cortex, parietal cortex, temporal cortex, occipital cortex and cerebellum. In some aspects, PLP1 expression is reduced in the cervical spinal cord, thoracic spinal cord, lumbar spinal cord, and/or lumbar dorsal root ganglion. In some aspects, the composition and/or the oligonucleotide does not comprise a targeting ligand. In some aspects, the oligonucleotide is not formulated in a lipid, liposome or lipid nanoparticle delivery vehicle. In some aspects, the pharmaceutically acceptable carrier comprises phosphate buffered saline.

In some aspects, the disclosure provides a method for reducing GFAP expression in the central nervous system of a subject, comprising administering an RNAi oligonucleotide described herein. In some aspects, GFAP mRNA expression, GFAP protein expression, or both, are reduced in the subject. In some aspects, the subject has astrogliosis and the reduction of GFAP expression reduces astrogliosis in the subject. In other aspects, the disclosure provides a method of reducing astrogliosis in a subject, comprising administering an RNAi oligonucleotide described herein. In some aspects, the disclosure provides a method of reducing demyelination in a subject, comprising administering an RNAi oligonucleotide described herein. In other aspects, the disclosure provides a method of reducing dysmyelination in a subject, comprising administering an RNAi oligonucleotide described herein.

In some aspects, the disclosure provides a method of determining responsiveness to treatment in a patient that has received or is receiving an RNA oligonucleotide treatment targeting PLP1, comprising: determining a level of GFAP expression in a sample from the patient, wherein reduction in the level of GFAP expression indicates responsiveness to treatment in the patient.

In other aspects, the disclosure provides a method of determining responsiveness to treatment in a patient with a disease, disorder or condition associated with PLP1 expression, comprising:
(i) administering an RNAi oligonucleotide treatment targeting PLP1 to the patient; and
(ii) determining a level of GFAP expression in a sample from the patient, wherein reduction in the level of GFAP expression indicates responsiveness to treatment in the patient.

In further aspects, the disclosure provides a method of determining responsiveness to treatment in a patient having astrogliosis, comprising:
(i) administering an RNAi oligonucleotide treatment targeting PLP1 to the patient; and
(ii) determining a level of GFAP expression in a sample from the patient, wherein reduction in the level of GFAP expression indicates responsiveness to treatment in the patient.

### BRIEF DESCRIPTION OF FIGURES

FIGS. 1A-1B provide graphs depicting the efficacy of oligonucleotides designed to inhibit murine PLP1 mRNA expression. The percent (%) of PLP1 mRNA remaining in CNS tissue was measured in C57BL/6 mice 7-days following intrathecal injection with 250 μg of a PLP1 oligonucleotide formulated in PBS relative to the % of PLP1 mRNA in PBS treated mice. FIG. 1A depicts the % of PLP1 mRNA in the lumbar spinal cord. FIG. 1B depicts the % of PLP1 mRNA in the frontal cortex.

FIGS. 2A-2D provide graphs depicting the dose response of 3 oligonucleotides selected based on inhibitory efficacy shown in FIGS. 1A-1B. The percent (%) of murine PLP1 mRNA remaining in CNS tissue was measured in C57BL/6 mice 7-days following intrathecal injection with 100 μg or 250 μg of indicated PLP1 oligonucleotides (PLP1-2339, PLP1-2398, and PLP1-2340; with the modification pattern described in Example 2, having a fully phosphothiolated loop) formulated in PBS relative to the % of PLP1 mRNA in PBS treated mice. % mRNA was determined in the lumbar spinal cord (FIG. 2A), brainstem (FIG. 2B), hippocampus (FIG. 2C), and frontal cortex (FIG. 2D).

FIG. 3 provides a schematic depicting the general structure and chemical modification pattern of N-Acetylgalactosamine (GalNAc)-conjugated double stranded RNAi (dsRNAi) oligonucleotides. 2′-OMe=2′-O-methyl; 2′-F=2′-fluoro.

FIGS. 4A-4D provide graphs depicting the dose response of 3 oligonucleotides selected based on inhibitory efficacy shown in FIGS. 1A-1B. The percent (%) of murine PLP1 mRNA remaining in CNS tissue was measured in C57BL/6 mice 7-days following intrathecal injection with 30 μg, 100 82 g, or 300 μg of indicated PLP1 oligonucleotides (PLP1-2339, PLP1-2398, and PLP1-2340; with the modification pattern depicted in FIG. 3, having a GalNAc-conjugated loop) formulated in PBS relative to the % of PLP1 mRNA in PBS treated mice. % mRNA was determined in the frontal cortex (FIG. 4A), cerebellum (FIG. 4B), brainstem (FIG. 4C), and lumbar spinal cord (FIG. 4D).

FIG. 5 provides a graph depicting the efficacy of oligonucleotides designed to inhibit human and/or non-human primate PLP1 mRNA expression using a hydrodynamic injection (HDI) model in CD-1 mice. 3 days after subcutaneous dosing of 3 mg/kg of PLP1 oligonucleotides conjugated to N-Acetylgalactosamine (GalNAc), designed to target exons 3-8, and formulated in PBS, a plasmid encoding human PLP1 mRNA was injected into the mice via HDI and the percent (%) of human PLP1 mRNA was measured 1 day later in liver samples from the mice relative to mice treated with PBS.

FIG. 6 provides a graph depicting the dose response of 6 oligonucleotides selected (targeting exons 3-5) based on inhibitory efficacy shown in FIG. 3. The same HDI model described in FIG. 5 was used, except doses of 0.3 mg/kg or 1 mg/kg were administered to the mice. The percent (%) of human PLP1 mRNA was measured in liver samples from the mice relative to mice treated with PBS.

FIG. 7 provides a schematic depicting the structure and modification pattern of an oligonucleotide having 2′-Fluoro and 2′-O-methyl modifications, including a 2′-O-methyl Tetraloop. 2′-OMe=2′-O-methyl, 2′-F=2′-fluoro.

FIGS. 8A-8C provide graphs depicting the percent (%) PLP1 mRNA remaining in mouse lumbar spinal cord after a single intrathecal 300 μg bolus dose of the indicated PLP1 oligonucleotides modified with a GalNAc tetraloop (as depicted in FIG. 3) or a 2′-O-methyl tetraloop (as depicted in FIG. 7). Tissue was collected at day 7 for PLP1-2340 (8A), day 28 for PLP1-2398 (8B), and day 56 for PLP1-2339 (8C) for PLP1 mRNA measurement. Artificial cerebral spinal fluid (aCSF), having no oligonucleotide, was used as a control.

FIG. 9A provides a graph depicting the percent (%) PLP1 mRNA remaining in non-human primates after a single dose (45 mg on day 0) or multidose (45 mg on day 0 and day 7) of PLP1-436 with the modification pattern depicted in FIG. 7. Animals were monitored throughout the study and tissue was collected at day 28 and day 84 for PLP1 mRNA measurement. Artificial cerebral spinal fluid (aCSF), having no oligonucleotide, was used as a control.

FIG. 9B provides images of whole brain in situ hybridization measuring PLP1 mRNA in non-human primates (treated as described in FIG. 9A) after a single dose (45 mg on day or multidose of PLP1-436 with the modification pattern depicted in FIG. 7. Artificial cerebral spinal fluid (aCSF), having no oligonucleotide, was used as a control.

FIG. 10 provides graphs depicting the dose response of PLP1-2340 (with the modification pattern depicted in FIG. 7). The percent (%) of murine PLP1 mRNA remaining in CNS tissue was measured in C57BL/6 mice 7-days following intracerebroventricular (i.c.v) injection with 10 μg, 30 μg, 100 μg, or 300 μg of oligonucleotide formulated in aCSF. Percent (%) remaining mRNA was determined in the frontal cortex, hippocampus, brain stem, and lumbar spinal cord. Artificial cerebral spinal fluid (aCSF), having no oligonucleotide, was used as a control.

FIGS. 11A-11B provide graphs depicting the dose response of PLP1-2340 (with the modification pattern depicted in FIG. 7) The percent (%) Plp1 mRNA remaining in CNS tissue was measured in C57BL/6 and Plp1-dup mice 7-days following i.c.v, injection with 30 μg, 100 μg, 300 μg, or 500 μg of oligonucleotide formulated in PBS. Percent (%) remaining mRNA was determined in the frontal cortex, somatosensory cortex, hippocampus (FIG. 11A), cerebellum, brain stem, and lumbar spinal cord (FIG. 11B). Artificial cerebral spinal fluid (aCSF), having no oligonucleotide, was used as a control.

FIGS. 12A-12E provide graphs depicting the percent (%) Plp1 mRNA remaining in C57BL/6 and Plp1-dup mice after a single 500 μg i.c.v. injection of PLP1-2340 (with the modification pattern depicted in FIG. 7). Tissue was collected at day 7, 14, 28, 56, and 84 for Plp1 mRNA measurement. Percent (%) remaining mRNA was determined in the frontal cortex (12A), hippocampus (12B), cerebellum (12C), brain stem (12D), and lumbar spinal cord (12E). Artificial cerebral spinal fluid (aCSF), having no oligonucleotide, was used as a control.

FIG. 13 provides immunofluorescent images of PLP1 protein expression in corpus callosum 56 days after i.c.v. administration of 500 μg of PLP1-2340 or aCSF in Plp1-dup mice. C57Bl/6 mice treated with aCSF were used as a control.

FIGS. 14A-14E provide graphs depicting the percent (%) Gfap mRNA remaining in C57BL/6 and Plp1-dup mice after a single 500 μg i.c.v. injection of PLP1-2340 (with the modification pattern depicted in FIG. 7). Tissue was collected at day 7, 14, 28, 56, and 84 for Gfap mRNA measurement. Percent (%) remaining mRNA was determined in the frontal cortex (14A), hippocampus (14B), cerebellum (14C), brain stem (14D), and lumbar spinal cord (14E). Artificial cerebral spinal fluid (aCSF), having no oligonucleotide, was used as a control.

FIG. 15 provides immunofluorescent images of GFAP protein expression in whole brain 56 days after i.c.v. administration of 500 μg of PLP1-2340 or aCSF in Plp1-dup mice. C57Bl/6 mice treated with aCSF were used as a control. *Black circle in C57Bl/6 (aCSF) is an artifact of DAPI staining.

FIGS. 16A-16C provide graphs depicting the dose response of PLP1-2340 (with the modification pattern depicted in FIG. 7) in neonatal (P4) mice. The percent (%) of murine Plp1 (FIG. 16A), Mbp (FIG. 16B), and Gfap (FIG. 16C) mRNA remaining in CNS tissue was measured in C57BL/6 mice 7-days following i.c.v injection with 10 μg, 30 μg, 100 μg, or 250 μg of oligonucleotide formulated in PBS. Percent (%) remaining mRNA was determined in the left hemisphere, right hemisphere, and spinal cord. Artificial cerebral spinal fluid (aCSF), having no oligonucleotide, was used as a control.

FIGS. 17A-17E provide graphs depicting the percent (%) Plp1 mRNA remaining in C57BL/6 and Plp1-dup mice after a single 250 μg i.c.v. injection of PLP1-2340 (with the modification pattern depicted in FIG. 7). Mice were injected at age P4 and tissue was collected at P28 for Plp1 mRNA measurement. Percent (%) remaining mRNA was determined in the frontal cortex (17A), hippocampus (17B), cerebellum (17C), brain stem (17D), and lumbar spinal cord (17E). Artificial cerebral spinal fluid (aCSF), having no oligonucleotide, was used as a control.

### DETAILED DESCRIPTION

According to some aspects, the disclosure provides oligonucleotides that reduce PLP1 expression in the central nervous system. In some embodiments, the oligonucleotides provided herein are designed to treat diseases associated with PLP expression in the CNS. In some respects, the disclosure provides methods of treating a disease associated with PLP expression by reducing PLP1 gene expression in cells (e.g., cells of the CNS).

### Oligonucleotide Inhibitors of PLP1 Expression

The disclosure provides, inter alia, oligonucleotides that inhibit PLP1 expression (e.g., RNAi oligonucleotides). In some embodiments, an oligonucleotide that inhibits PLP expression is targeted to a PLP1 mRNA.

### PLP1 Target Sequences

In some embodiments, the oligonucleotide is targeted to a target sequence comprising a PLP1 mRNA. In some embodiments, the oligonucleotide, or a portion, fragment or strand thereof (e.g., an antisense strand or a guide strand of a double-stranded oligonucleotide) binds or anneals to a target sequence comprising a PLP1 mRNA, thereby inhibiting PLP1 expression. In some embodiments, the oligonucleotide is targeted to a PLP1 target sequence for the purpose of inhibiting PLP1 expression in vivo. In some embodiments, the amount or extent of inhibition of PLP1 expression by an oligonucleotide targeted to a PLP1 target sequence correlates with the potency of the oligonucleotide. In some embodiments, the amount or extent of inhibition of PLP1 expression by an oligonucleotide targeted to a PLP1 target sequence correlates with the amount or extent of therapeutic benefit in a subject or patient having a disease, disorder or condition associated with PLP1 expression treated with the oligonucleotide.

Through examination of the nucleotide sequence of mRNAs encoding PLP1, including mRNAs of multiple different species (e.g., human, cynomolgus monkey, mouse, and rat, see e.g., Example 1) and as a result of in vitro and in vivo testing (see. e.g., Examples 2-5), it has been discovered that certain nucleotide sequences of PLP1 mRNA are more amenable than others to oligonucleotide-based-inhibition and are thus useful as target sequences for the oligonucleotides herein. In some embodiments, a sense strand of an oligonucleotide (e.g., a double-stranded oligonucleotide) described herein comprises a PLP1 target sequence. In some embodiments, a portion or region of the sense strand of a double-stranded oligonucleotide described herein comprises a PLP1 target sequence. In some embodiments, a PLP1 target sequence comprises, or consists of, a nucleotide sequence of any one of SEQ ID NOs: 171-188. In some embodiments, a PLP1 target sequence comprises, or consists of, a nucleotide sequence of any one of SEQ ID Nos: 212-231. In some embodiments, a PLP1 target sequence comprises the nucleotide sequence set forth in SEQ ID NO: 171. In some embodiments, a PLP1 target sequence comprises the nucleotide sequence set forth in SEQ ID NO: 212. In some embodiments, a PLP1 target sequence comprises the nucleotide sequence set forth in SEQ ID NO: 219. In some embodiments, a PLP1 target sequence comprises the nucleotide sequence set forth in SEQ ID NO: 224. In some embodiments, a PLP1 target sequence comprises the nucleotide sequence set forth in SEQ ID NO: 215. In some embodiments, a PLP1 target sequence comprises the nucleotide sequence set forth in SEQ ID NO: 213. In some embodiments, a PLP1 target sequence comprises the nucleotide sequence set forth in SEQ ID NO: 220.

### PLP1-Targeting Sequences

In some embodiments, the oligonucleotides herein have regions of complementarity to PLP1 mRNA (e.g., within a target sequence of PLP1 mRNA) for purposes of targeting the mRNA in cells and inhibiting its expression. In some embodiments, the oligonucleotides herein comprise a PLP1 targeting sequence (e.g., an antisense strand or a guide strand of a double-stranded oligonucleotide) having a region of complementarity that binds or anneals to a PLP1 target sequence by complementary (Watson-Crick) base pairing. The targeting sequence or region of complementarity is generally of suitable length and base content to enable binding or annealing of the oligonucleotide (or a strand thereof) to a PLP1 mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is at least 12, at least 13, at least 14, at least 15, at least 16, at least 17, at least 18, at least 19 or at least 20 nucleotides. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, an oligonucleotide comprises a target sequence or region of complementarity complementary to a sequence of any one of SEQ ID NOs: 212-231, and the targeting sequence or region of complementarity is 18 nucleotides in length. In some embodiments, an oligonucleotide comprises a target sequence or region of complementarity complementary to a sequence of any one of SEQ ID NOs: 212-231, and the targeting sequence or region of complementarity is 19 nucleotides in length.

In some embodiments, an oligonucleotide herein comprises a targeting sequence or a region of complementarity (e.g., an antisense strand or a guide strand of a double-stranded oligonucleotide) that is fully complementary to a PLP1 target sequence. In some embodiments, the targeting sequence or region of complementarity is partially complementary to a PLP1 target sequence. In some embodiments, the oligonucleotide comprises a targeting sequence or region of complementarity that is fully complementary to a sequence of any one of SEQ ID NOs: 76, 78, 82, 84, 86, 88, 90, 92, 94, 96, 98, 100, 102, 104, 106, 108 and 110. In some embodiments, the oligonucleotide comprises a targeting sequence or region of complementarity that is partially complementary to a sequence of any one of SEQ ID NOs: 76, 78, 80, 82, 84, 86, 88, 90, 92, 94, 96, 98, 100, 102, 104, 106, 108 and 110. In some embodiments, the oligonucleotide comprises a targeting sequence or region of complementarity that is fully complementary to a sequence of any one of SEQ ID NOs: 212-231. In some embodiments, the oligonucleotide comprises a targeting sequence or region of complementarity that is fully complementary to the sequence set forth in SEQ ID NO: 212. In some embodiments, the oligonucleotide comprises a targeting sequence or region of complementarity that is fully complementary to the sequence set forth in SEQ ID NO: 212, 219, 224, 215, 213 or 220. In some embodiments, the oligonucleotide comprises a targeting sequence or region of complementarity that is partially complementary to a sequence of any one of SEQ ID NOs: 212-231. In some embodiments, the oligonucleotide comprises a targeting sequence or region of complementarity that is partially complementary to the sequence of SEQ ID NO: 212. In some embodiments, the oligonucleotide comprises a targeting sequence or region of complementarity that is partially complementary to the sequence of SEQ ID NO: 212, 219, 224, 215, 213 or 220.

In some embodiments, the oligonucleotide herein comprises a targeting sequence or region of complementarity that is complementary to a contiguous sequence of nucleotides comprising an PLP1 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising an PLP1 mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising an PLP1 mRNA, wherein the contiguous sequence of nucleotides is 19 nucleotides in length. In some embodiments, the oligonucleotide comprises a targeting sequence or region of complementarity that is complementary to a contiguous sequence of nucleotides comprising an PLP1 mRNA, wherein the contiguous sequence of nucleotides is 20 nucleotides in length. In some embodiments, the oligonucleotide comprises a targeting sequence or a region of complementarity that is complementary to a contiguous sequence of nucleotides of any one of SEQ ID NOs: 76, 78, 80, 82, 84, 86, 88, 90, 92, 94, 96, 98, 100, 102, 104, 106, 108 and 110, optionally wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 212-231, optionally wherein the contiguous sequence of nucleotides is 19 nucleotides in length. In some embodiments, the oligonucleotide comprises a targeting sequence or a region of complementarity that is complementary to a contiguous sequence of nucleotides of SEQ ID NO: 212, optionally wherein the contiguous sequence of nucleotides is 19 nucleotides in length. In some embodiments, the oligonucleotide comprises a targeting sequence or a region of complementarity that is complementary to a contiguous sequence of nucleotides of SEQ ID NO: 212, 219, 224, 215, 213 or 220, optionally wherein the contiguous sequence of nucleotides is 19 nucleotides in length.

In some embodiments, a targeting sequence or region of complementarity of an oligonucleotide is complementary to contiguous nucleotides of a sequence as set forth in any one of SEQ ID NOs: 76, 78, 80, 82, 84, 86, 88, 90, 92, 94, 96, 98, 100, 102, 104, 106, 108 and 110 and spans the entire length of an antisense strand. In some embodiments, a region of complementarity of an oligonucleotide is complementary to contiguous nucleotides of a sequence as set forth in any one of SEQ ID NOs: 76, 78, 80, 82, 84, 86, 88, 90, 92, 94, 96, 98, 100, 102, 104, 106, 108 and 110 and spans a portion of the entire length of an antisense strand. In some embodiments, an oligonucleotide herein comprises a region of complementarity (e.g., on an antisense strand of a double-stranded oligonucleotide) that is at least partially (e.g., fully) complementary to a contiguous stretch of nucleotides spanning nucleotides 1-20 of a sequence as set forth in any one of SEQ ID NOs: 76, 78, 80, 82, 84, 86, 88, 90, 92, 94, 96, 98, 100, 102, 104, 106, 108 and 110. In some embodiments, a targeting sequence or region of complementarity of an oligonucleotide is complementary to contiguous nucleotides of a sequence as set forth in any one of SEQ ID NOs: 212-23 land spans the entire length of an antisense strand. In some embodiments, a region of complementarity of an oligonucleotide is complementary to contiguous nucleotides of a sequence as set forth in any one of SEQ ID NOs: 212-231 and spans a portion of the entire length of an antisense strand. In some embodiments, an oligonucleotide herein comprises a region of complementarity (e.g., on an antisense strand of a double-stranded oligonucleotide) that is at least partially (e.g., fully) complementary to a contiguous stretch of nucleotides spanning nucleotides 1-19 of a sequence as set forth in any one of SEQ ID NOs: 212-231.

In some embodiments, an oligonucleotide herein comprises a targeting sequence or region of complementarity having one or more base pair (bp) mismatches with the corresponding PLP1 target sequence. In some embodiments, the targeting sequence or region of complementarity may have up to about 1, up to about 2, up to about 3, up to about 4, up to about 5, etc. mismatches with the corresponding PLP1 target sequence provided that the ability of the targeting sequence or region of complementarity to bind or anneal to the PLP1 mRNA under appropriate hybridization conditions and/or the ability of the oligonucleotide to inhibit PLP1 expression is maintained. Alternatively, in some embodiments, the targeting sequence or region of complementarity comprises no more than 1, no more than 2, no more than 3, no more than 4, or no more than 5 mismatches with the corresponding PLP1 target sequence provided that the ability of the targeting sequence or region of complementarity to bind or anneal to the PLP1 mRNA under appropriate hybridization conditions and/or the ability of the oligonucleotide to inhibit PLP1 expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence or region of complementarity. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

In some embodiments, the oligonucleotide comprises a targeting sequence or a region of complementary that is complementary to a contiguous sequence of nucleotides of any one of SEQ ID NOs: 212-231, wherein the targeting sequence or region of complementarity may have up to about 1, up to about 2, up to about 3, up to about 4, up to about 5, etc. mismatches with the corresponding PLP1 target sequence. In some embodiments, the oligonucleotide comprises a targeting sequence or a region of complementary that is complementary to a contiguous sequence of nucleotides of any one of SEQ ID NOs: 212-231, wherein the targeting sequence or region of complementarity may have no more than 1, no more than 2, no more than 3, no more than 4, or no more than 5 mismatches with the corresponding PLP1 target sequence. In some embodiments, the oligonucleotide comprises a targeting sequence or a region of complementary that is complementary to a contiguous sequence of nucleotides of SEQ ID NO: 212, wherein the targeting sequence or region of complementarity may have up to about 1, up to about 2, up to about 3, up to about 4, up to about 5, etc. mismatches with the corresponding PLP1 target sequence. In some embodiments, the oligonucleotide comprises a targeting sequence or a region of complementary that is complementary to a contiguous sequence of nucleotides of SEQ ID NO: 212, wherein the targeting sequence or region of complementarity may have no more than 1, no more than 2, no more than 3, no more than 4, or no more than 5 mismatches with the corresponding PLP1 target sequence.

### Types of Oligonucleotides

A variety of oligonucleotide types and/or structures are useful for targeting PLP1 mRNA in the methods herein including, but not limited to, RNAi oligonucleotides, antisense oligonucleotides, miRNAs, etc. Any of the oligonucleotide types described herein or elsewhere are contemplated for use as a framework to incorporate an PLP1 mRNA targeting sequence herein for the purposes of inhibiting PLP1 expression.

In some embodiments, the oligonucleotides herein inhibit PLP1 expression by engaging with RNA interference (RNAi) pathways upstream or downstream of Dicer involvement (e.g., an RNAi oligonucleotide). For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include single-stranded extensions (on one or both sides of the molecule) as well as double-stranded extensions.

In some embodiments, the oligonucleotides herein engage with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target mRNA (e.g., PLP1 mRNA) and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides disclosed herein comprise sense and antisense strands that are both in the range of about 17 to 26 (e.g., 17 to 26, 20 to 25 or 21-23) nucleotides in length. In some embodiments, an oligonucleotide disclosed herein comprises a sense and antisense strand that are both in the range of about 19-22 nucleotides in length. In some embodiments, the sense and antisense strands are of equal length. In some embodiments, an oligonucleotide disclosed herein comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY, Blackburn (ed.), Royal Society of Chemistry, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-732), fork siRNAs (see, e.g., Hohjoh (2004) FEBS LETT. 557:193-98), single-stranded siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structure that may be used in some embodiments to reduce or inhibit the expression of PLP1 are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, an oligonucleotide for reducing or inhibiting PLP1 expression herein is single-stranded (ss). Such structures may include but are not limited to single-stranded RNAi molecules. Recent efforts have demonstrated the activity of single-stranded RNAi molecules (see, e.g., Matsui et al. (2016) Mol. Ther. 24:946-955). However, in some embodiments, oligonucleotides herein are antisense oligonucleotides (ASOs). An antisense oligonucleotide is a single-stranded oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) Annu. Rev. Pharmacol. 57:81-105).

### Double-Stranded RNAi Oligonucleotides

In some aspects, the disclosure provides double-stranded (ds) RNAi oligonucleotides for targeting PLP1 mRNA and inhibiting PLP1 expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a tetraloop or triloop (L), and a second subregion (S2), wherein L is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 19 nucleotides in length. In some embodiments, D1 is 20 nucleotides in length. In some embodiments, D1 comprising the sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, a dsRNAi oligonucleotide provided herein comprises a sense strand having a sequence of any one of SEQ ID NOs: 76, 78, 80, 82, 84, 86, 88, 90, 92, 94, 96, 98, 100, 102, 104, 106, 108 and 110 and an antisense strand comprising a complementary sequence of any one of SEQ ID NOs: 77, 79, 81, 83, 85, 87, 89, 91, 93, 95, 97, 99, 101, 103, 105, 107, 109 and 111, as arranged in Table 5. In some embodiments, the sense strand comprises the sequence of SEQ ID NO: 76 and the antisense strand comprises the sequence of SEQ ID NO: 77.

In some embodiments, a dsRNAi oligonucleotide comprises a sense strand and an antisense strand comprising sequence selected from:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

It should be appreciated that, in some embodiments, sequences presented in the Sequence Listing may be referred to in describing the structure of an oligonucleotide (e.g., a dsRNAi oligonucleotide) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a dsRNAi oligonucleotide herein comprises a 25-nucleotide sense strand and a 27-nucleotide antisense strand that when acted upon by a Dicer enzyme results in an antisense strand that is incorporated into the mature RISC. In some embodiments, the sense strand of the dsRNAi oligonucleotide is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the dsRNAi oligonucleotide is longer than 25 nucleotides (e.g., 26, 27, 28, 29 or 30 nucleotides).

In some embodiments, the dsRNAi oligonucleotides herein have one 5′ end that is thermodynamically less stable when compared to the other 5′ end. In some embodiments, an asymmetric dsRNAi oligonucleotide is provided that comprises a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, a dsRNAi oligonucleotide has a two-nucleotide overhang on the 3′ end of the antisense (guide) strand. However, other overhangs are possible. In some embodiments, an overhang is a 3 ′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., PLP1 mRNA). In some embodiments, the two terminal nucleotides on the 3′ end of the antisense strand are not complementary with the target mRNA. In some embodiments, the two terminal nucleotides on the 3′ end of the antisense strand of a dsRNAi oligonucleotide herein are unpaired. In some embodiments, the two terminal nucleotides on the 3′ end of the antisense strand of a dsRNAi oligonucleotide herein comprise an unpaired GG. In some embodiments, the two terminal nucleotides on the 3′ end of the antisense strand of a dsRNAi oligonucleotide herein are not complementary to the target mRNA. In some embodiments, two terminal nucleotides on each 3′ end of a dsRNAi oligonucleotide are GG. Typically, one or both of the two terminal GG nucleotides on each 3′ end of a double-stranded oligonucleotide is not complementary with the target mRNA.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of the dsRNAi oligonucleotide improves or increases the potency of the dsRNAi oligonucleotide.

### Antisense Strands

In some embodiments, an antisense strand of a dsRNAi oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, a dsRNAi oligonucleotide herein comprises an antisense strand of up to about 50 nucleotides in length (e.g., up to 50, up to 40, up to 35, up to 30, up to 27, up to 25, up to 21, up to 19, up to 17, up to 15, or up to 12 nucleotides in length). In some embodiments, a dsRNAi oligonucleotide comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, a dsRNAi oligonucleotide comprises an antisense strand in a range of about 12 to about 40 (e.g., 12 to 40, 12 to 36, 12 to 32, 12 to 28, 15 to 40, 15 to 36, 15 to 32, 15 to 30, 15 to 28, 17 to 22, 17 to 19 to 27, 19 to 30, 20 to 40, 22 to 40, 25 to 40 or 32 to 40) nucleotides in length. In some embodiments, a dsRNAi oligonucleotide comprises an antisense of 15 to 30 nucleotides in length. In some embodiments, an antisense strand of any one of the dsRNAi oligonucleotides disclosed herein is of 12, 13, 14, 15, 16, 17, 18, 19, 20, 21, 22, 23, 24, 25, 26, 27, 28, 29, 30, 31, 32, 33, 34, 36, 37, 38, 39 or 40 nucleotides in length. In some embodiments, an dsRNAi oligonucleotide comprises an antisense strand of 22 nucleotides in length.

In some embodiments, a dsRNAi oligonucleotide disclosed herein for targeting PLP1 mRNA and inhibiting PLP1 expression comprises an antisense strand comprising a sequence as set forth in any one of SEQ ID NOs: 77, 79, 81, 83, 85, 87, 89, 91, 93, 95, 97, 99, 101, 103, 105, 107, 109 and 111. In some embodiments, a dsRNAi oligonucleotide herein comprises an antisense strand comprising at least about 12 (e.g., at least 12, at least 13, at least 14, at least at least 16, at least 17, at least 18, at least 19, at least 20, at least 21, at least 22 or at least 23) contiguous nucleotides of a sequence as set forth in any one of SEQ ID NOs: 77, 79, 81, 83, 85, 87, 89, 91, 93, 95, 97, 99, 101, 103, 105, 107, 109 and 111.

### Sense Strands

In some embodiments, a dsRNAi oligonucleotide disclosed herein for targeting PLP1 mRNA and inhibiting PLP1 expression comprises a sense strand sequence as set forth in in any one of SEQ ID NOs: 76, 78, 80, 82, 84, 86, 88, 90, 92, 94, 96, 98, 100, 102, 104, 106, 108 and 110. In some embodiments, a dsRNAi oligonucleotide has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 76, 78, 80, 82, 84, 86, 88, 90, 92, 94, 96, 98, 100, 102, 104, 106, 108 and 110.

In some embodiments, a dsRNAi oligonucleotide herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, a dsRNAi oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, to 40 or 32 to 40) nucleotides in length. In some embodiments, a dsRNAi oligonucleotide comprises a sense strand 15 to 50 nucleotides in length. In some embodiments, a dsRNAi oligonucleotide comprises a sense strand 18 to 36 nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a dsRNAi oligonucleotide comprises a sense strand of 36 nucleotides in length.

In some embodiments, a sense strand comprises a stem-loop structure at its 3′ end. In some embodiments, the stem-loop is formed by intrastrand base pairing. In some embodiments, a sense strand comprises a stem-loop structure at its 5′ end. In some embodiments, a stem is a duplex of 2, 3, 4, 5, 6, 7, 8, 9, 10, 11, 12, 13 or 14 nucleotides in length. In some embodiments, a stem-loop provides the dsRNAi oligonucleotide protection against degradation (e.g., enzymatic degradation), facilitates or improves targeting and/or delivery to a target cell, tissue, or organ (e.g., the liver), or both. For example, in some embodiments, the loop of a stem-loop provides nucleotides comprising one or more modifications that facilitate, improve, or increase targeting to a target mRNA (e.g., an PLP1 mRNA), inhibition of target gene expression (e.g., PLP1 expression), and/or delivery to a target cell, tissue, or organ (e.g., the CNS), or a combination thereof. In some embodiments, the stem-loop itself or modification(s) to the stem-loop do not substantially affect the inherent gene expression inhibition activity of the dsRNAi oligonucleotide, but facilitates, improves, or increases stability (e.g., provides protection against degradation) and/or delivery of the oligonucleotide to a target cell, tissue, or organ (e.g., the CNS). In certain embodiments, a dsRNAi oligonucleotide comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length.

In some embodiments, a loop (L) of a stem-loop having the structure S1-L-S2 as described above is a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Ends

In some embodiments, a dsRNAi oligonucleotide herein comprises sense and antisense strands, such that there is a 3′-overhang on either the sense strand or the antisense strand, or both the sense and antisense strand. In some embodiments, a dsRNAi oligonucleotide provided herein has one 5′ end that is thermodynamically less stable compared to the other 5′ end. In some embodiments, an asymmetric dsRNAi oligonucleotide is provided that includes a blunt end at the 3′ end of a sense strand and overhang at the 3′ end of the antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two (2) nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides. In some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides. In some embodiments, the 3′ overhang comprises purine nucleotides. In some embodiments, the 3′ overhang is selected from AA, GG, AG and GA. In some embodiments, the 3′ overhang is GG or AA. In some embodiments, the 3′ overhang is GG.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 2′ end of an antisense strand are complementary with the target. In some embodiments, the last one or two nucleotides at the 3′ end of the antisense strand are not complementary with the target.

In some embodiments, a dsRNAi oligonucleotide herein comprises a step-loop structure at the 3′ end of the sense strand and comprises two terminal overhang nucleotides at the 3′ end of the antisense strand. In some embodiments, a dsRNAi oligonucleotide herein comprises a nicked tetraloop structure, wherein the 3′ end sense strand comprises a stem-tetraloop structure and comprises two terminal overhang nucleotides at the 3′ end of antisense strand. In some embodiments, the two terminal overhang nucleotides are GG. Typically, one or both of the two terminal GG nucleotides of the antisense strand are not complementary with the target.

In some embodiments, the 5′ end and/or the 3′ end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 2′ end or 5′ end of a sense and/or antisense strand.

### Oligonucleotide Modifications

In some embodiments, a dsRNAi oligonucleotide described herein comprises a modification. Oligonucleotides (e.g., dsRNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, the modification is a reversible modification. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, at least one modified base, and at least one reversible modification.

The number of modifications on an oligonucleotide (e.g., a dsRNAi oligonucleotide) and the position of those nucleotide modifications may influence the properties of an oligonucleotide. For example, oligonucleotides may be delivered in vivo by conjugating them to encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

In some embodiments, a sense strand described here is 36 nucleotides in length and positions are numbered 1-36 from 5′ to 3′. In some embodiments, an antisense strand described herein is 22 nucleotides in length and positions are numbered 1-22 from 5′ to 3′. In some embodiments, position numbers described herein adhere to this numbering format.

### Sugar Modifications

In some embodiments, a dsRNAi oligonucleotide described herein comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a dsRNAi oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the dsRNAi oligonucleotide comprises at least about 1 modified nucleotide (e.g., at least 1, at least 5, at least 10, at least 15, at least 20, at least 25, at least 30, at least 35, or more). In some embodiments, the antisense strand of the dsRNAi oligonucleotide comprises at least about 1 modified nucleotide (e.g., at least 1, at least 5, at least 10, at least 15, at least 20, or more).

In some embodiments, all the nucleotides of the sense strand of the dsRNAi oligonucleotide are modified. In some embodiments, all the nucleotides of the antisense strand of the dsRNAi oligonucleotide are modified. In some embodiments, all the nucleotides of the dsRNAi oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid). In some embodiments, the modified nucleotide comprises a 2′-modification (e.g., a 2′-F or 2′-OMe)

In some embodiments, the disclosure provides dsRNAi oligonucleotides having different modification patterns. In some embodiments, the modified dsRNAi oligonucleotides comprise a sense strand sequence having a modification pattern as set forth in the Examples and Sequence Listing and an antisense strand having a modification pattern as set forth in the Examples and Sequence Listing.

In some embodiments, a dsRNAi oligonucleotide disclosed herein comprises an antisense strand having nucleotides that are modified with 2′-F. In some embodiments, a dsRNAi oligonucleotide disclosed herein comprises an antisense strand comprises nucleotides that are modified with 2′-F and 2′-OMe. In some embodiments, a dsRNAi oligonucleotide disclosed herein comprises a sense strand having nucleotides that are modified with 2′-F. In some embodiments, a dsRNAi oligonucleotide disclosed herein comprises a sense strand comprises nucleotides that are modified with 2′-F and 2′-OMe.

In some embodiments, a dsRNAi oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2-fluoro modification. In some embodiments, a dsRNAi oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the dsRNAi oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the dsRNAi oligonucleotide comprise a 2′-fluoro modification.

In some embodiments, for these oligonucleotides, one or more of positions 8, 9, 10 or 11 of the sense strand is modified with a T-F group. In some embodiments, for these oligonucleotides, the sugar moiety at each of nucleotides at positions 1-7 and 12-20 in the sense strand is modified with a 2′-OMe. In some embodiments, for these oligonucleotides, the sugar moiety at each of nucleotides at positions 1-7 and 12-36 in the sense strand is modified with a 2′-OMe.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7 and 10 of the antisense strand are modified with a 2′-F. In some embodiments, the sugar moiety at positions 2, 5 and 14 and optionally up to 3 of the nucleotides at positions 3, 4, 7 and 10 of the antisense strand are modified with a 2′-F. In other embodiments, the sugar moiety at each of the positions at positions 2, 5 and 14 of the antisense strand is modified with the 2′-F. In other embodiments, the sugar moiety at each of the positions at positions 1, 2, 5 and 14 of the antisense strand is modified with the 2′-F. In other embodiments, the sugar moiety at each of the positions at positions 2, 4, 5 and 14 of the antisense strand is modified with the 2′-F. In still other embodiments, the sugar moiety at each of the positions at positions 1, 2, 3, 5, 7 and 14 of the antisense strand is modified with the 2′-F. In other embodiments, the sugar moiety at each of the positions at positions 2, 3, 4, 5, 7 and 14 of the antisense strand is modified with the 2′-F. In yet another embodiment, the sugar moiety at each of the positions at positions 1, 2, 3, 5, 10 and 14 of the antisense strand is modified with the 2′-F. In other embodiments, the sugar moiety at each of the positions at positions 2, 3, 4, 5, 10 and 14 of the antisense strand is modified with the 2′-F. In another embodiment, the sugar moiety at each of the positions at positions 2, 3, 5, 7, 10 and 14 of the antisense strand is modified with the 2′-F. In other embodiments, the sugar moiety at each of the positions at positions 2, 3, 4, 5, 7, and 14 of the antisense strand is modified with the 2′-F.

In some embodiments, an oligonucleotide provided herein comprises an antisense strand having the sugar moiety at positions 2 and 14 modified with 2′-F. In some embodiments, an oligonucleotide provided herein comprises an antisense strand having the sugar moiety at positions 2, 5, and 14 modified with 2′-F. In some embodiments, an oligonucleotide provided herein comprises an antisense strand having the sugar moiety at positions 1, 2, 5, and 14 modified with 2′-F. In some embodiments, an oligonucleotide provided herein comprises an antisense strand having the sugar moiety at positions 2, 4, 5, and 14 modified with 2′-F. In some embodiments, an oligonucleotide provided herein comprises an antisense strand having the sugar moiety at positions 1, 2, 3, 5, 7, and 14 modified with 2′-F. In some embodiments, an oligonucleotide provided herein comprises an antisense strand having the sugar moiety at positions 2, 3, 4, 5, 7, and 14 modified with 2′-F. In some embodiments, an oligonucleotide provided herein comprises an antisense strand having the sugar moiety at positions 1, 2, 3, 5, 10, and 14 modified with 2′-F. In some embodiments, an oligonucleotide provided herein comprises an antisense strand having the sugar moiety at positions 2, 3, 4, 5, 10, and 14 modified with 2′-F. In some embodiments, an oligonucleotide provided herein comprises an antisense strand having the sugar moiety at positions 2, 3, 4, 5, 7, 10, and 14 modified with 2′-F.

In some embodiments, an oligonucleotide provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 4, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at positions 8-11 modified with 2′-F. In some embodiments, an oligonucleotide provided herein comprises a sense strand having the sugar moiety at positions 1-7 and 12-17 or 12-20 modified with 2′ OMe. In some embodiments, an oligonucleotide provided herein comprises a sense strand having the sugar moiety at positions 1-7 and 12-17, 12-20 or 12-22 modified with 2′OMe. In some embodiments, an oligonucleotide provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, an oligonucleotide provided herein comprises a sense strand having the sugar moiety of each of the nucleotides at positions 1-7 and 12-17, 12-20 or 12-22 of the sense strand modified with a modification selected from the group consisting of 2′-O-propargyl, 2′-O-propylamin, 2′-amino, 2′-ethyl, 2′-aminoethyl (EA), 2′-(2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position position 31, position 32, position 33, position 34, position 35, or position 36 modified with 2′-F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position position 31, position 32, position 33, position 34, position 35, or position 36 modified with 2′-OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 4, 5, 7, 10 and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA); and a sense strand having the sugar moiety at each of the nucleotides at positions 8-11 of the sense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, the sense and antisense strands of an oligonucleotide comprise nucleotides sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively,
   wherein one or more of positions 8, 9, 10 or 11 of the sense strand is modified with a 2′-F group.

### 5′-Terminal Phosphate

In some embodiments, an oligonucleotide described herein comprises a 5′-terminal phosphate. In some embodiments, 5′-terminal phosphate groups of an RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, an oligonucleotide (e.g., a double-stranded oligonucleotide) herein includes analogs of 5′ phosphates that are resistant to such degradation. In some embodiments, the phosphate analog is oxymethylphosphonate, vinylphosphonate or malonylphosphonate, or a combination thereof. In certain embodiments, the 5′ end of an oligonucleotide strand is attached to chemical moiety that mimics the electrostatic and steric properties of a natural 5′-phosphate group (“phosphate mimic”).

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si (CH₃)₃ or a protecting group. In certain embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy. In some embodiments, the 4′-phosphate analog is 4′-oxymethylphoshonate. In some embodiments, the modified nucleotide having the 4′-phosphonate analog is a uridine. In some embodiments, the modified nucleotide is 4′-O-monomethylphosphonate-2′-O-methyl uridine.

In some embodiments, the sense and antisense strands of an oligonucleotide comprise nucleotides sequences selected from the group consisting of
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively,
   wherein the oligonucleotide comprises a 5′-terminal phosphate, optionally a 5′-terminal phosphate analog.

In some embodiments, a dsRNAi oligonucleotide provided herein comprises an antisense strand comprising a 4′-phosphate analog at the 5′-terminal nucleotide, wherein 5′-terminal nucleotide comprises the following structure:

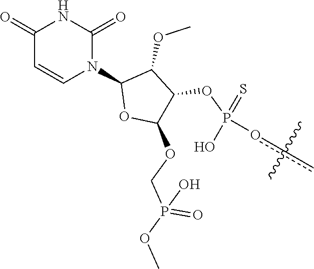

4′-monomethylphosphonate-2′-O-methyluridine phosphorothioate

### [MePhosphonate-40-mUs]

### Modified Internucleoside Linkage

In some embodiments, an oligonucleotide (e.g., a dsRNAi oligonucleotide) herein comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an oligonucleotide provided herein (e.g., a dsRNAi oligonucleotide) has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of (i) positions 1 and 2 of the sense strand; and (ii) positions 1 and 2, positions 2 and 3, positions 3 and 4, positions and 21, and positions 21 and 22 of the antisense strand.

In some embodiments, the sense and antisense strands of an oligonucleotide comprise nucleotides sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively, wherein the oligonucleotide comprises a modified internucleotide linkage.

### Base Modifications

In some embodiments, oligonucleotides herein (e.g., dsRNAi oligonucleotides) have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1 position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrol e (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target the oligonucleotides of the disclosure (e.g., dsRNAi oligonucleotides) to one or more cells or one or more organs. Such a strategy can help to avoid undesirable effects in other organs or avoid undue loss of the oligonucleotide to cells, tissue or organs that would not benefit from the oligonucleotide. Accordingly, in some embodiments, oligonucleotides disclosed herein (e.g., dsRNAi oligonucleotides) are modified to facilitate targeting and/or delivery to a particular tissue, cell, or organ (e.g., to facilitate delivery of the oligonucleotide to the CNS). In some embodiments, an oligonucleotide comprises at least one nucleotide (e.g., 1, 2, 3, 4, 5, 6 or more nucleotides) conjugated to one or more targeting ligand(s).

In some embodiments, the targeting ligand comprises a carbohydrate, amino sugar, cholesterol, peptide, polypeptide, protein or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a ds oligonucleotide) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g. human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (L) has a monovalent GalNAc moiety attached to any one or more guanine nucleotides of the tetraloop via any linker described herein, as depicted below (X=heteroatom):

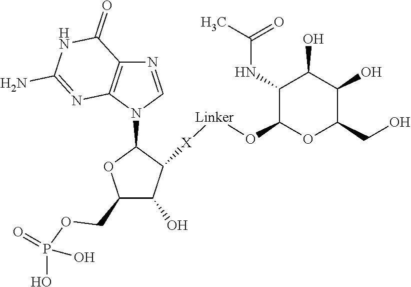

In some embodiments, the tetraloop (L) has a monovalent GalNAc attached to any one or more adenine nucleotides of the tetraloop via any linker described herein, as depicted below (X=heteroatom):

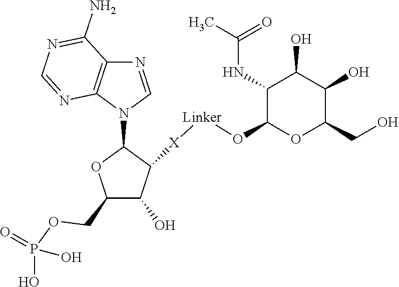

In some embodiments, an oligonucleotide herein comprises a monovalent GalNAc attached to a guanine nucleotide referred to as [ademG-GalNAc] or 2′-aminodiethoxymethanol-Guanine-GalNAc, as depicted below:

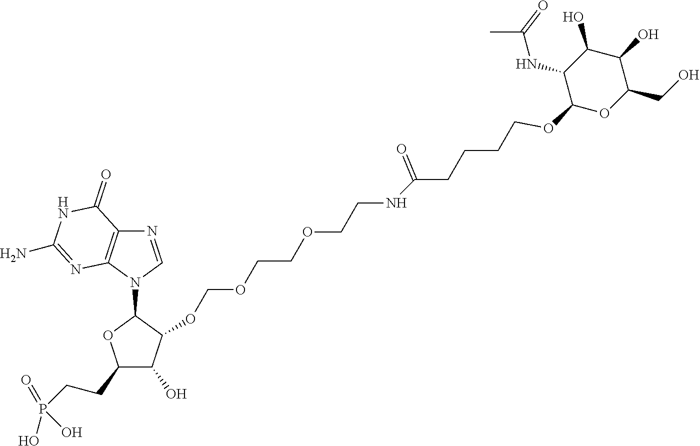 [SMILES: CC(=O)N[C@H]1[C@H](OCCCCC(=O)NCCOCCOCO[C@@H]2[C@H](O)[C@@H](CCP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

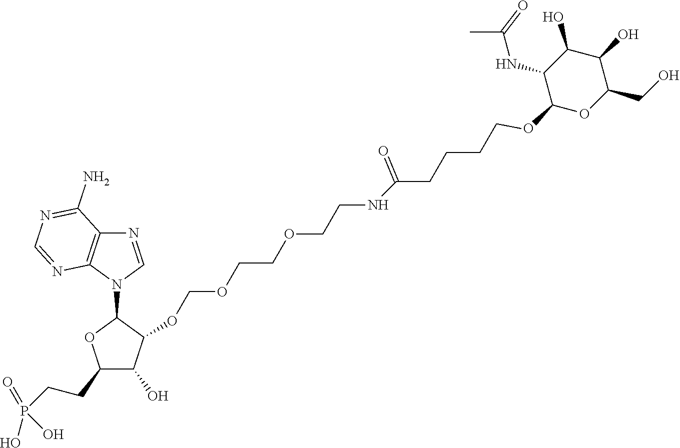

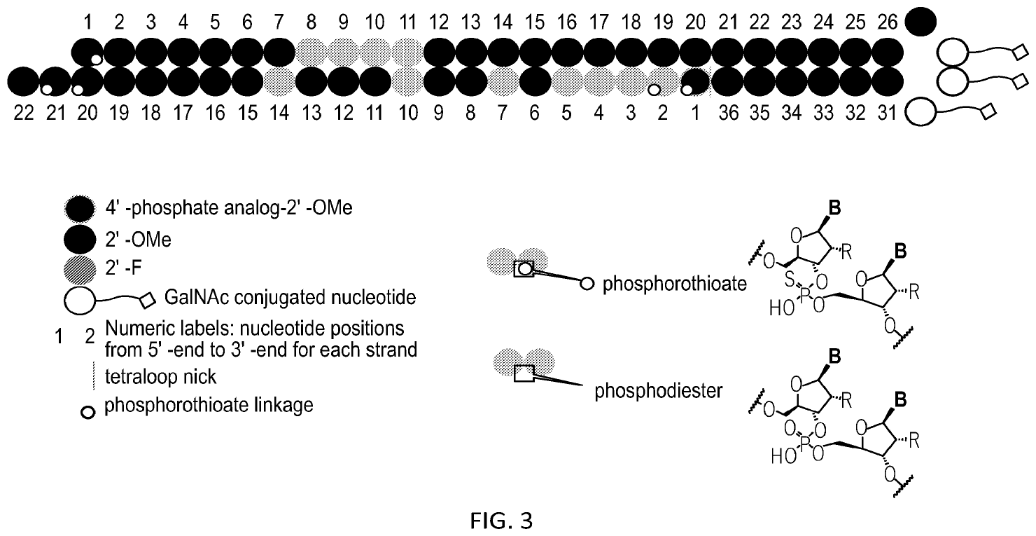

An example of such conjugation is shown below for a loop comprising from 5′ to 3′ the nucleotide sequence GAAA (L=linker, X=heteroatom) stem attachment points are shown. Such a loop may be present, for example, at positions 27-30 of the sense strand listed in Table 5 and as shown in FIG. 3. In the chemical formula,

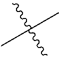

is used to describe an attachment point to the oligonucleotide strand.

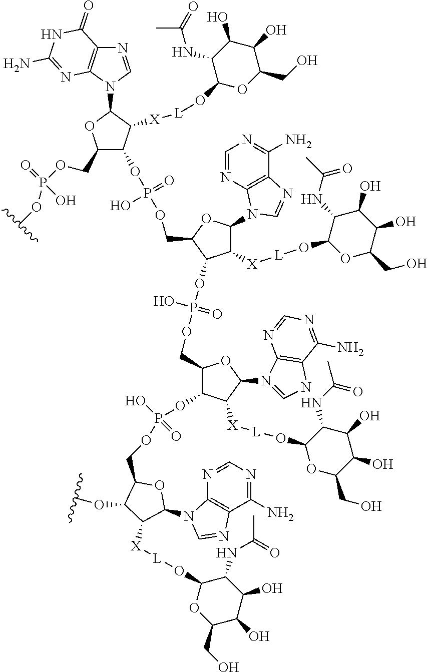

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to 3 or 4 nucleotides of the loop using an acetal linker. Such a loop may be present, for example, at positions 27-30 of the any one of the sense strand listed in Table 5 and as shown in FIG. 3. In the chemical formula,

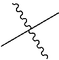

is an attachment point to the oligonucleotide strand.

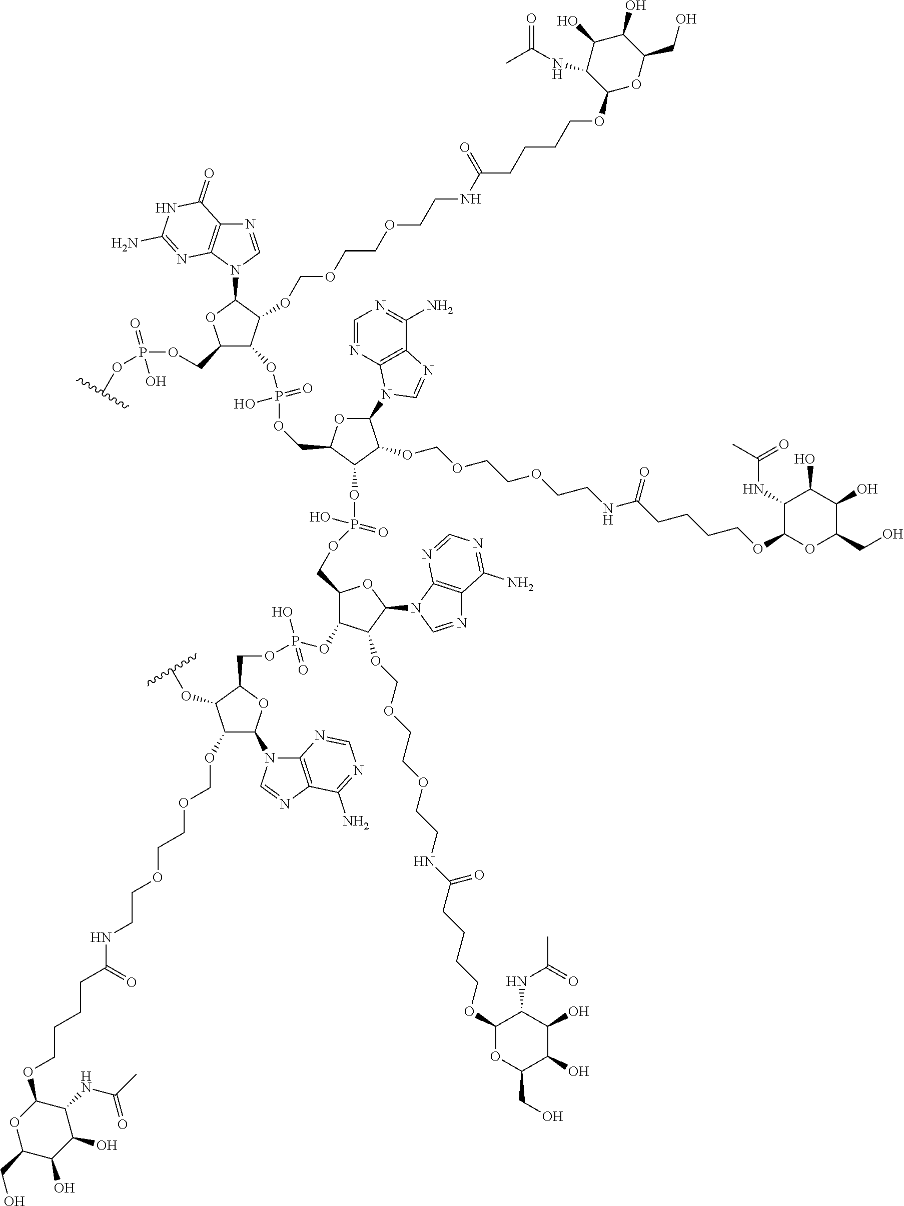 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

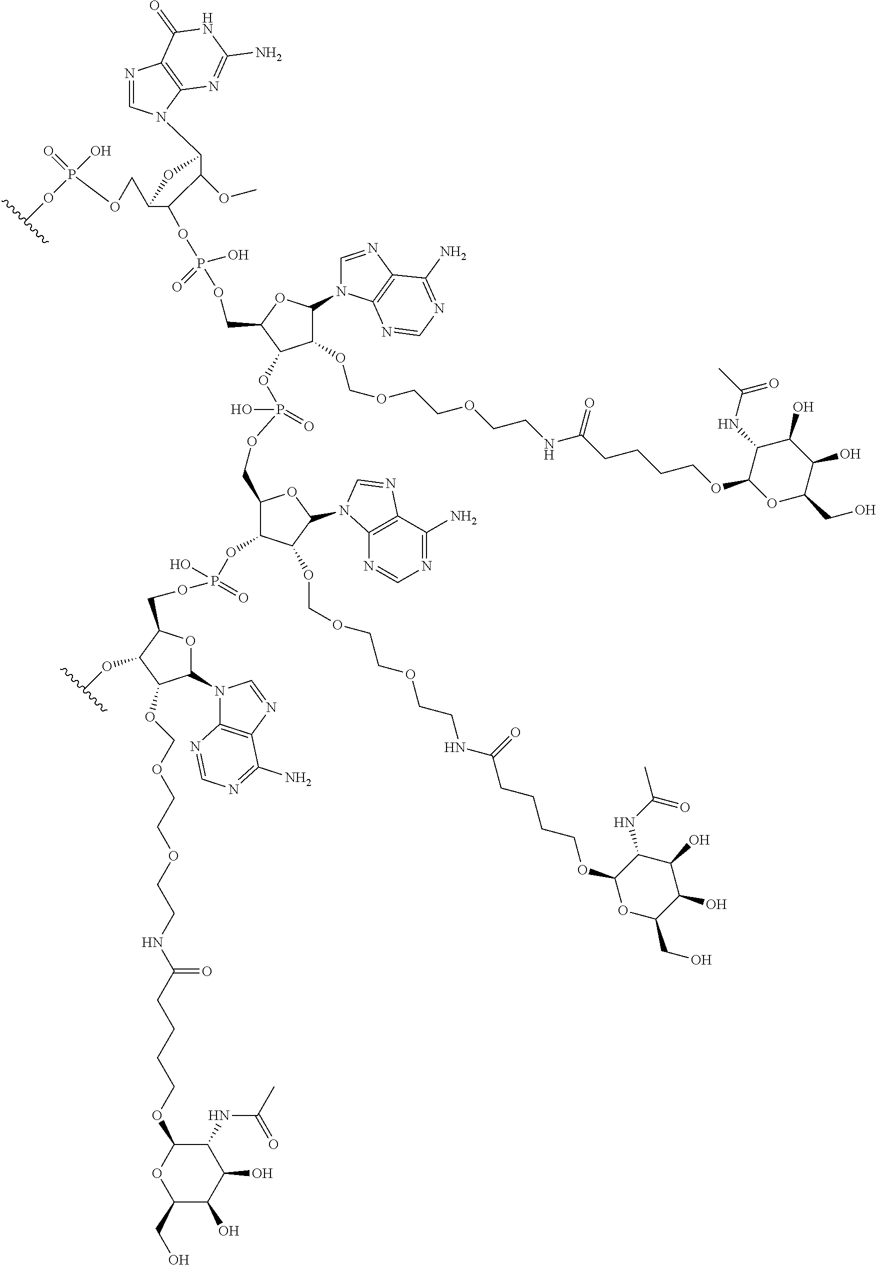

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a dsRNAi oligonucleotide. In some embodiments, the oligonucleotides herein (e.g., dsRNAi oligonucleotides) do not have a GalNAc conjugated thereto.

Exemplary PLP1-Targeting dsRNAi Oligonucleotides

In some embodiments, the disclosure provides dsRNAi oligonucleotides that target PLP1 mRNA and reduce PLP1 expression (referred to herein as PLP1-targeting dsRNAi oligonucleotides), wherein the oligonucleotides comprise sense strand and an antisense strand that form a duplex region, and wherein the antisense strand comprises a region of complementarity to a PLP1 mRNA target sequence of any one of SEQ ID NOs: 171-188, and wherein the region of complementarity is at least 15 contiguous nucleotides in length. In some embodiments, the region of complementarity is 15-20 nucleotides in length. In some embodiments, the region of complementarity is 15 nucleotides, 16 nucleotides, 17 nucleotides, 18 nucleotides, 19 nucleotides, or 20 nucleotides in length. In some embodiments, the region of complementarity is at least 19 contiguous nucleotides in length. In some embodiments, the region of complementary is at least 20 nucleotides in length. In some embodiments, the region of complementarity is 19 nucleotides in length. In some embodiments, the region of complementarity is 20 nucleotides in length. In some embodiments, the PLP1 mRNA target sequence comprises any one of SEQ ID Nos: 212-231.

In some embodiments, the sense strand is 15 to 50 nucleotides in length. In some embodiments, the sense strand is 18 to 36 nucleotides in length. In some embodiments, the sense strand is 36 nucleotides in length. In some embodiments, the antisense strand is 15 to 30 nucleotides in length. In some embodiments, the antisense strand is 22 nucleotides in length. In some embodiments, the sense strand is 36 nucleotides in length and the antisense strand is 22 nucleotides in length and the sense and antisense strand form a duplex region that is at least 19 nucleotides in length. In some embodiments, the duplex region is 20 nucleotides in length.

In some embodiments, the PLP1-targeting dsRNAi oligonucleotides for reducing PLP1 expression provided by the disclosure comprises a stem-loop set forth as S1-L-S2, wherein S1 is complementary to S2, and wherein L forms a loop between S1 and S2 of 3-5 nucleotides in length. In some embodiments, S1 and S2 are 1-10 nucleotides in length and are the same length. In some embodiments, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some embodiments, S1 and S2 are 6 nucleotides in length. In some embodiments the loop is 3 nucleotides in length. In some embodiments, the loop is 4 nucleotides in length. In some embodiments, the loop is 5 nucleotides in length. In some embodiments, L is a triloop or a tetraloop. In some embodiments, L is a triloop. In some embodiments, L is a tetraloop. In some embodiments, the tetraloop comprises the sequence 5′-GAAA-3′. In some embodiments, the stem loop comprises the sequence 5′-GCAGCCGAAAGGCUGC-3′ (SEQ ID NO: 190). In some embodiments, up to 4 nucleotides comprising L are each conjugated to a targeting ligand. In some embodiments, 1 nucleotide, 2 nucleotides, 3 nucleotides, or 4 nucleotides comprising L are each conjugated to a targeting ligand. In some embodiments, 3 nucleotides comprising L are each conjugated to a targeting ligand. In some embodiments, L is a tetraloop comprising the sequence 5′-GAAA-3′, wherein each adenosine (A) nucleoside comprising the tetraloop is conjugated to a targeting ligand comprising a monovalent N-acetylgalactosamine (GalNAc) moiety.

In some embodiments, each nucleotide in the tetraloop is a 2′-O-methyl modified nucleotide. In some embodiments, the tetraloop comprises the sequence 5′-GAAA-3′ with each nucleotide comprising a 2′-O-methyl modification.

In some embodiments, each nucleotide in the tetraloop of an oligonucleotide is a 2′-O-methyl modified nucleotide, and the oligonucleotide does not comprise a targeting ligand and/or is not formulated in a delivery vehicle (e.g., lipid nanoparticle). In some embodiments, the oligonucleotide comprises a tetraloop comprising the sequence 5′-GAAA-3′ with each nucleotide comprising a 2′-O-methyl modification, and the oligonucleotide does not comprise a targeting ligand and/or is not formulated in a delivery vehicle (e.g., lipid nanoparticle).

In some embodiments, the antisense strand comprises a 3′ overhang sequence of one or more nucleotides in length. In some embodiments, the 3′ overhang sequence is two (2) nucleotides in length. In some embodiments, the 3′ overhang comprises purine nucleotides. In some embodiments, the sequence of the 3′ over hang is 5′-AA-3′, 5′-GG-3′, 5′-AG-3′ or 5′-GA-3′. In some embodiments, the sequence of the 3′ overhang is 5′-GG-3′.

In some embodiments, the PLP1-targeting dsRNAi oligonucleotides for reducing PLP1 expression provided by the disclosure comprise sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

In some embodiments, the PLP1-targeting dsRNAi oligonucleotides for reducing PLP1 expression provided by the disclosure comprises at least one modified nucleotide. In some embodiments, the modified nucleotide comprises a five (5) carbon sugar (e.g., ribose) with a 2′-modification. In some embodiments, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the 2′-modification is 2′-fluoro or 2′-O-methyl. In some embodiments, all nucleotides comprising the PLP1-targeting dsRNAi oligonucleotides are modified. In some embodiments, all nucleotides comprising the PLP1-targeting dsRNAi oligonucleotides are modified with a 2′-modification selected from 2′-fluoro and 2′-O-methyl.

In some embodiments, the PLP1-targeting dsRNAi oligonucleotides comprises at least one modified internucleotide linkage. In some embodiments, the at least one modified internucleotide linkage is a phosphorothioate linkage.

In some embodiments, the PLP1-targeting dsRNAi oligonucleotides comprise an antisense strand wherein the 4′-carbon of the sugar of the 5′-terminal nucleotide of the antisense strand comprises a phosphate analog. In some embodiments, the phosphate analog is oxymethylphosphonate, vinylphosphonate or malonylphosphonate. In some embodiments, the phosphate analog is a 4′-phosphate analog comprising 5′-methoxyphosphonate-4′-oxy. In some embodiments, the phosphate analog is a 4′-phosphate analog comprising 4′-oxymethylphosphonate.

In some embodiments, the PLP1-targeting dsRNAi oligonucleotide for reducing PLP1 expression provided by the disclosure comprise a sense strand and an antisense strand, wherein all nucleotides comprising the sense strand and antisense strand are modified, wherein the antisense strand comprises a region of complementarity to a PLP1 mRNA target sequence of any one of SEQ ID NOs: 171-188, and wherein the region of complementarity is at least 15 contiguous nucleotides in length. In some embodiments, the 5′-terminal nucleotide of the antisense strand comprises 5′-methoxyphosphonate-4′-oxy-2′-O-methyluridine [MePhosphonate-40-mU], as described herein. In some embodiments, the 5′-terminal nucleotide of the antisense strand comprises a phosphorothioate linkage. In some embodiments, the antisense strand and the sense strand comprise one or more 2′-fluoro (2′-F) and 2′-O-methyl (2′-OMe) modified nucleotides and at least one phosphorothioate linkage. In some embodiments, the antisense strand comprises four (4) phosphorothioate linkages and the sense strand comprises one (1) phosphorothioate linkage.

In some embodiments, the PLP1 targeting dsRNAi oligonucleotide comprises
(i) an antisense strand of 19-30 nucleotides in length, wherein the antisense strand comprises a nucleotide sequence comprising a region of complementarity to a PLP1 mRNA target sequence, wherein the region of complementarity is selected from SEQ ID NOs: 235-254, and
(ii) a sense strand of 19-50 nucleotides in length comprising a region of complementarity to the antisense strand, wherein the antisense and sense strands are separate strands which form an asymmetric duplex region having an overhang of 1-4 nucleotides at the 3′ terminus of the antisense strand.

In some embodiments, the PLP1 targeting dsRNAi oligonucleotide comprises
(i) an antisense strand of 19-30 nucleotides in length, wherein the antisense strand comprises a nucleotide sequence comprising a region of complementarity to a PLP1 mRNA target sequence, wherein the region of complementarity is selected from SEQ ID NOs: 235-254, and
(ii) a sense strand of 19-50 nucleotides in length comprising a region of complementarity to the antisense strand, wherein the sense strand comprises as its 3′ end a stem-loop set forth as: S1-L-S2, wherein S1 is complementary to S2, and wherein L forms a loop between S1 and S2 of 3 to 5 nucleotides in length, wherein the antisense and sense strands are separate strands which form an asymmetric duplex region having an overhang of 1-4 nucleotides at the 3′ terminus of the antisense strand.

In some embodiments, the PLP1 targeting dsRNAi oligonucleotide comprises
(i) an antisense strand of 19-30 nucleotides in length, wherein the antisense strand comprises a nucleotide sequence comprising a region of complementarity to a PLP1 mRNA target sequence, wherein the region of complementarity is selected from SEQ ID NOs: 235-254, and
(ii) a sense strand of 19-50 nucleotides in length comprising a region of complementarity to the antisense strand, wherein the sense strand comprises as its 3′ end a stem-loop set forth as: S1-L-S2, wherein S1 is complementary to S2, and wherein L forms a loop between S1 and S2 of 3 to 5 nucleotides in length, wherein L is comprised of 2′-OMe modified nucleotides, wherein the antisense and sense strands are separate strands which form an asymmetric duplex region having an overhang of 1-4 nucleotides at the 3′ terminus of the antisense strand.

In some embodiments, the PLP1-targeting dsRNAi oligonucleotides for reducing PLP1 expression comprise:
a sense strand comprising a 2′-F modified nucleotide at positions 8-11, a 2′-OMe modified nucleotide at positions 1-7, 12-26, and 31-36, a GalNAc-conjugated nucleotide at position 27, 28, and 29; and a phosphorothioate linkage between positions 1 and 2;
an antisense strand comprising a 2′-F modified nucleotide at positions 2, 3, 5, 7, 10 and 14, a 2′-OMe at positions 1, 4, 6, 8, 9, 11-13, and 15-22, a phosphorothioate linkage between positions 1 and 2, positions 2 and 3, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand for a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 comprise an overhang, and wherein the sense strand and antisense strands comprise nucleotide sequences selected from the group consisting of:
   (a) SEQ ID NOs: 76 and 77, respectively
   (b) SEQ ID NOs: 78 and 79, respectively;
   (c) SEQ ID NOs: 80 and 81, respectively;
   (d) SEQ ID NOs: 82 and 83, respectively;
   (e) SEQ ID NOs: 84 and 85, respectively;
   (f) SEQ ID NOs: 86 and 87, respectively;
   (g) SEQ ID NOs: 88 and 89, respectively;
   (h) SEQ ID NOs: 90 and 91, respectively;
   (i) SEQ ID NOs: 92 and 93, respectively;
   (j) SEQ ID NOs: 94 and 95, respectively;
   (k) SEQ ID NOs: 96 and 97, respectively;
   (l) SEQ ID NOs: 98 and 99, respectively;
   (m) SEQ ID NOs: 100 and 101, respectively;
   (n) SEQ ID NOs: 102 and 103, respectively;
   (o) SEQ ID NOs: 104 and 105, respectively;
   (p) SEQ ID NOs: 106 and 107, respectively;
   (q) SEQ ID NOs: 108 and 109, respectively; and
   (r) SEQ ID NOs: 110 and 111, respectively.

In some embodiments, the PLP1-targeting dsRNAi oligonucleotides for reducing PLP1 expression comprise:
a sense strand comprising a 2′-F modified nucleotide at positions 8-11, a 2′-OMe modified nucleotide at positions 1-7, 12-26, and 31-36, a GalNAc-conjugated nucleotide at position 27, 28, and 29; and a phosphorothioate linkage between positions 1 and 2;
an antisense strand comprising a 2′-F modified nucleotide at positions 2, 3, 4, 5, 7, and 14, a 2′-OMe at positions 1, 6, 8, 9, 11-13, and 15-22, a phosphorothioate linkage between positions 1 and 2, positions 2 and 3, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand for a duplex region with positions 1-20 of the sense strand, wherein positions 21-36 of the sense strand form a stem-loop, wherein positions 27-form the loop of the stem-loop, optionally wherein positions 27-30 comprise a tetraloop, wherein positions 21 and 22 comprise an overhang, and wherein the sense strand and antisense strands comprise nucleotide sequences selected from the group consisting of:
   (a) SEQ ID NOs: 76 and 77, respectively
   (b) SEQ ID NOs: 78 and 79, respectively;
   (c) SEQ ID NOs: 80 and 81, respectively;
   (d) SEQ ID NOs: 82 and 83, respectively;
   (e) SEQ ID NOs: 84 and 85, respectively;
   (f) SEQ ID NOs: 86 and 87, respectively;
   (g) SEQ ID NOs: 88 and 89, respectively;
   (h) SEQ ID NOs: 90 and 91, respectively;
   (i) SEQ ID NOs: 92 and 93, respectively;
   (j) SEQ ID NOs: 94 and 95, respectively;
   (k) SEQ ID NOs: 96 and 97, respectively;
   (l) SEQ ID NOs: 98 and 99, respectively;
   (m) SEQ ID NOs: 100 and 101, respectively;
   (n) SEQ ID NOs: 102 and 103, respectively;
   (o) SEQ ID NOs: 104 and 105, respectively;
   (p) SEQ ID NOs: 106 and 107, respectively;
   (q) SEQ ID NOs: 108 and 109, respectively; and
   (r) SEQ ID NOs: 110 and 111, respectively.

In some embodiments, the PLP1-targeting dsRNAi oligonucleotides for reducing PLP1 expression comprise:
a sense strand comprising a 2′-F modified nucleotide at positions 8-11, a 2′-OMe modified nucleotide at positions 1-7, and 12-36; and a phosphorothioate linkage between positions 1 and 2;
an antisense strand comprising a 2′-F modified nucleotide at positions 2, 3, 4, 5, 7, and 14, a 2′-OMe at positions 1, 6, 8, 9, 11-13, and 15-22, a phosphorothioate linkage between positions 1 and 2, positions 2 and 3, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand for a duplex region with positions 1-20 of the sense strand, wherein positions 21-36 of the sense strand form a stem-loop, wherein positions 27-form the loop of the stem-loop, optionally wherein positions 27-30 comprise a tetraloop, wherein positions 21 and 22 comprise an overhang, and wherein the sense strand and antisense strands comprise nucleotide sequences selected from the group consisting of:
   (a) SEQ ID NOs: 76 and 77, respectively
   (b) SEQ ID NOs: 78 and 79, respectively;
   (c) SEQ ID NOs: 80 and 81, respectively;
   (d) SEQ ID NOs: 82 and 83, respectively;
   (e) SEQ ID NOs: 84 and 85, respectively;
   (f) SEQ ID NOs: 86 and 87, respectively;
   (g) SEQ ID NOs: 88 and 89, respectively;
   (h) SEQ ID NOs: 90 and 91, respectively;
   (i) SEQ ID NOs: 92 and 93, respectively;
   (j) SEQ ID NOs: 94 and 95, respectively;
   (k) SEQ ID NOs: 96 and 97, respectively;
   (l) SEQ ID NOs: 98 and 99, respectively;
   (m) SEQ ID NOs: 100 and 101, respectively;
   (n) SEQ ID NOs: 102 and 103, respectively;
   (o) SEQ ID NOs: 104 and 105, respectively;
   (p) SEQ ID NOs: 106 and 107, respectively;
   (q) SEQ ID NOs: 108 and 109, respectively; and
   (r) SEQ ID NOs: 110 and 111, respectively.

In some embodiments, the PLP1-targeting dsRNAi oligonucleotides for reducing PLP1 expression comprise:
a sense strand comprising a 2′-F modified nucleotide at positions 8-11, a 2′-OMe modified nucleotide at positions 1-7, and 12-36; and a phosphorothioate linkage between positions 1 and 2;
an antisense strand comprising a 2′-F modified nucleotide at positions 2, 3, 4, 5, 7, and 14, a 2′-OMe at positions 1, 6, 8, 9, 11-13, and 15-22, a phosphorothioate linkage between positions 1 and 2, positions 2 and 3, positions 3 and 4, positions 20 and 21, and positions 21 and 22, and a 5′-terminal nucleotide at position 1 comprising a 4′-phosphate analog, optionally wherein the 5′-terminal nucleotide comprises 4′-O-monomethylphosphonate-2′-O-methyluridine [MePhosphonate-40-mU]; wherein positions 1-20 of the antisense strand for a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 comprise an overhang, and wherein the sense strand and antisense strands comprise nucleotide sequences selected from the group consisting of:
   (a) SEQ ID NOs: 76 and 77, respectively
   (b) SEQ ID NOs: 78 and 79, respectively;
   (c) SEQ ID NOs: 80 and 81, respectively;
   (d) SEQ ID NOs: 82 and 83, respectively;
   (e) SEQ ID NOs: 84 and 85, respectively;
   (f) SEQ ID NOs: 86 and 87, respectively;
   (g) SEQ ID NOs: 88 and 89, respectively;
   (h) SEQ ID NOs: 90 and 91, respectively;
   (i) SEQ ID NOs: 92 and 93, respectively;
   (j) SEQ ID NOs: 94 and 95, respectively;
   (k) SEQ ID NOs: 96 and 97, respectively;
   (l) SEQ ID NOs: 98 and 99, respectively;
   (m) SEQ ID NOs: 100 and 101, respectively;
   (n) SEQ ID NOs: 102 and 103, respectively;
   (o) SEQ ID NOs: 104 and 105, respectively;
   (p) SEQ ID NOs: 106 and 107, respectively;
   (q) SEQ ID NOs: 108 and 109, respectively; and
   (r) SEQ ID NOs: 110 and 111, respectively.

In some embodiments, a PLP1-targeting dsRNAi oligonucleotide for reducing PLP1 expression provided by the disclosure comprises a sense strand comprising the nucleotide sequence as set forth in SEQ ID NO: 76 and an antisense strand comprising the nucleotide sequence as set forth in SEQ ID NO: 77.

In some embodiments, a PLP1-targeting dsRNAi oligonucleotide for reducing PLP1 expression provided by the disclosure comprises a sense strand comprising the nucleotide sequence as set forth in SEQ ID NO: 88 and an antisense strand comprising the nucleotide sequence as set forth in SEQ ID NO: 89. In some embodiments, a PLP1-targeting dsRNAi oligonucleotide for reducing PLP1 expression provided by the disclosure comprises a sense strand comprising the nucleotide sequence as set forth in SEQ ID NO: 96 and an antisense strand comprising the nucleotide sequence as set forth in SEQ ID NO: 97. In some embodiments, a PLP1-targeting dsRNAi oligonucleotide for reducing PLP1 expression provided by the disclosure comprises a sense strand comprising the nucleotide sequence as set forth in SEQ ID NO: 80 and an antisense strand comprising the nucleotide sequence as set forth in SEQ ID NO: 81. In some embodiments, a PLP1-targeting dsRNAi oligonucleotide for reducing PLP1 expression provided by the disclosure comprises a sense strand comprising the nucleotide sequence as set forth in SEQ ID NO: 78 and an antisense strand comprising the nucleotide sequence as set forth in SEQ ID NO: 79. In some embodiments, a PLP1-targeting dsRNAi oligonucleotide for reducing PLP1 expression provided by the disclosure comprises a sense strand comprising the nucleotide sequence as set forth in SEQ ID NO: 90 and an antisense strand comprising the nucleotide sequence as set forth in SEQ ID NO: 91.

In some embodiments, PLP1-targeting dsRNAi oligonucleotide for reducing PLP1 expression comprises the following modification pattern:

Sense strand:
[mXs][mX][mX][mX][mX][mX][mX][fX][fX][fX][fX][mX][mX][mX][mX][mX][mX][mX][mX][mX]
[mX][mX][mX][mX][mX][mX][mX][mX][mX][mX][mX][mX][mX][mX][mX][mX]
Antisense strand:
[MePhosphonate-40-
mXs][fXs][fX][fX][fX][fX][fX][mX][mX][fX][mX][mX][mX][fX][mX][mX][mX][mX][mX][mXs]
[mXs][mX]
Modification key: Table 4
In some embodiments, PLP1-targeting dsRNAi oligonucleotide for reducing PLP1
expression comprises the following modification pattern:
Sense strand:
[mXs][mX][mX][mX][mX][mX][mX][fX][fX][fX][fX][mX][mX][mX][mX][mX][mX][mX][mX][mX]
[mX][mX][mX][mX][mX][mX][mX][mX][mX][mX][mX][mX][mX][mX][mX][mX]
Antisense strand:
[MePhosphonate-40-
mXs][fXs][fXs][fX][fX][mX][fX][mX][mX][mX][mX][mX][mX][mX][mX][mX][mX][mX][mX][mXs]
[mXs][mX]
Modification key: Table 4

In some embodiments, a PLP1-targeting dsRNAi oligonucleotide for reducing PLP1 expression provided by the disclosure comprising a sense strand selected from SEQ ID NOs: 112, 114, 116, 118, 120, 122, 124, 126, 128, 130, 132, 134, 136, 138, 140, 142, 144, 146, and 191 and an antisense strand selected from SEQ ID NOs: 113, 115, 117, 119, 121, 123, 125, 127, 129, 131, 133, 135, 137, 139, 141, 143, 145, 147, and 192. In some embodiments, a PLP1-targeting dsRNAi oligonucleotide for reducing PLP1 expression provided by the disclosure comprising a sense strand selected from SEQ ID NOs: 112, 114, 116, 118, 120, 122, 124, 126, 128, 130, 132, 134, 136, 138, 140, 142, 144, 146, and 191 and an antisense strand selected from SEQ ID NOs: 113, 115, 117, 119, 121, 123, 125, 127, 129, 131, 133, 135, 137, 139, 141, 143, 145, 147, and 207.

In some embodiments, a PLP1-targeting dsRNAi oligonucleotide for reducing PLP1 expression provided by the disclosure comprises a sense strand and an antisense strand, wherein the sense and antisense strand are selected from:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 132 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 192, respectively.

In some embodiments, a PLP1-targeting dsRNAi oligonucleotide for reducing PLP1 expression provided by the disclosure comprises a sense strand and an antisense strand, wherein the sense and antisense strand are selected from:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(l) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 132 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 207, respectively.

In some embodiments, PLP1-targeting dsRNAi oligonucleotide for reducing PLP1 expression comprises a sense strand comprising SEQ ID NO: 112, and an antisense strand comprising SEQ ID NO: 113.

In some embodiments, PLP1-targeting dsRNAi oligonucleotide for reducing PLP1 expression comprises a sense strand comprising SEQ ID NO: 191, and an antisense strand comprising SEQ ID NO: 192.

In some embodiments, PLP1-targeting dsRNAi oligonucleotide for reducing PLP1 expression comprises a sense strand comprising SEQ ID NO: 191, and an antisense strand comprising SEQ ID NO: 207.

In some embodiments, PLP1-targeting dsRNAi oligonucleotide for reducing PLP1 expression comprises a sense strand comprising SEQ ID NO: 124, and an antisense strand comprising SEQ ID NO: 125. In some embodiments, PLP1-targeting dsRNAi oligonucleotide for reducing PLP1 expression comprises a sense strand comprising SEQ ID NO: 132, and an antisense strand comprising SEQ ID NO: 133. In some embodiments, PLP1-targeting dsRNAi oligonucleotide for reducing PLP1 expression comprises a sense strand comprising SEQ ID NO: 116, and an antisense strand comprising SEQ ID NO: 117. In some embodiments, PLP1-targeting dsRNAi oligonucleotide for reducing PLP1 expression comprises a sense strand comprising SEQ ID NO: 114, and an antisense strand comprising SEQ ID NO: 115. In some embodiments, PLP1-targeting dsRNAi oligonucleotide for reducing PLP1 expression comprises a sense strand comprising SEQ ID NO: 126, and an antisense strand comprising SEQ ID NO: 127.

### Formulations

Various formulations have been developed to facilitate oligonucleotide use. For example, oligonucleotides (e.g., dsRNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., dsRNAi oligonucleotides) reduce the expression of PLP1. Such compositions can be suitably formulated such that when administered to a subject, either into the immediate environment of a target cell or systemically, a sufficient portion of the oligonucleotides enter the cell to reduce PLP1 expression. Any variety of suitable oligonucleotide formulations can be used to deliver oligonucleotides for the reduction of PLP1 as disclosed herein. In some embodiments, an oligonucleotide is formulated in buffer solutions such as phosphate buffered saline solutions, liposomes, micellar structures and capsids. In some embodiments, an oligonucleotide is formulated in buffer solutions such as phosphate buffered saline solutions.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions. In some embodiments, an oligonucleotide is not formulated with a component to facilitate transfection into cells.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

In some embodiments, a pharmaceutical composition is formulated for administration into the central nervous system. In some embodiments, a pharmaceutical composition is formulated for administration into the cerebral spinal fluid. In some embodiments, a pharmaceutical composition is formulated for administration to the spinal cord. In some embodiments, a pharmaceutical composition is formulated for intrathecal administration. In some embodiments, a pharmaceutical composition is formulated for administration to the brain. In some embodiments, a pharmaceutical composition is formulated for intracerebroventricular administration. In some embodiments, a pharmaceutical composition is formulated for the brain stem. In some embodiments, a pharmaceutical composition is formulated for intracisternal magna administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a dsRNAi oligonucleotide for reducing PLP1 expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing PLP1 Expression

In some embodiments, the disclosure provides methods for contacting or delivering to a cell or population of cells an effective amount of any of the oligonucleotides (e.g. dsRNAi oligonucleotides) herein to reduce PLP1 expression. In some embodiments, a reduction of PLP1 expression is determined by measuring a reduction in the amount or level of PLP1 mRNA, PLP1 protein, or PLP1 activity in a cell. The methods include those described herein and known to one of ordinary skill in the art.

In some embodiments, the disclosure provides methods for reducing PLP1 expression in the central nervous system. In some embodiments, the central nervous system comprises the brain and spinal cord. In some embodiments, PLP1 expression is reduced in at least one region of the brain. In some embodiments, regions of the brain include the frontal cortex, parietal cortex, temporal cortex, occipital cortex, and cerebellum. In some embodiments, regions of the brain include the frontal cortex, cerebellum, hippocampus, lumbar spinal cord, and brain stem. In some embodiments, regions of the brain include the frontal cortex, parietal cortex, temporal cortex, occipital cortex, cerebellum, hippocampus, lumbar spinal cord, and brain stem. In some embodiments, PLP1 expression is reduced in at least one region of the spinal cord. In some embodiments, regions of the spinal cord include the cervical spinal cord, thoracic spinal cord, lumbar spinal cord, and lumbar dorsal root ganglion. In some embodiments, PLP1 expression is reduced in at least one region of the brain and at least one region of the spinal cord. In some embodiments, PLP1 expression is reduced in at least one of the lumbar spinal cord, thoracic spinal cord, cervical spinal cord, brainstem, frontal cortex, parietal cortex occipital cortex. In some embodiments, PLP1 expression is reduced in at least one of the lumbar spinal cord, thoracic spinal cord, and cervical spinal cord.

In some embodiments, PLP1 expression is reduced for 1-12 weeks after administration of an oligonucleotide described herein. In some embodiments, PLP1 expression is reduced for 1, 2, 3, 4, 5, 6, 7, 8, 9, 10, 11 or 12 weeks after administration of an oligonucleotide described herein. In some embodiments, PLP1 expression is reduced for 1-4 months after administration of an oligonucleotide described herein. In some embodiments, PLP1 expression is reduced for 1-6 months after administration of an oligonucleotide described herein. In some embodiments, PLP1 expression is reduced for 1, 2, 3 or 4 months after administration of an oligonucleotide described herein. In some embodiments, PLP1 expression is reduced for 1, 2, 3 4, 5 or 6 months after administration of an oligonucleotide described herein. In some embodiments, PLP1 expression is reduced for 7-91 days after administration of an oligonucleotide described herein. In some embodiments, PLP1 expression is reduced for 7, 14, 21, 28, 35, 42, 49, 56, 63, 70, 77, 84 or 91 days after administration of an oligonucleotide described herein.

In some embodiments, PLP1 expression is reduced in at least one region of the brain and/or at least one region of the spinal cord for 1-12 weeks after administration of an oligonucleotide described herein. In some embodiments, PLP1 expression is reduced in at least one region of the brain and/or at least one region of the spinal cord for 1, 2, 3, 4, 5, 6, 7, 8, 9, 10, 11 or 12 weeks after administration of an oligonucleotide described herein. In some embodiments, PLP1 expression is reduced in at least one region of the brain and/or at least one region of the spinal cord for 1-4 months after administration of an oligonucleotide described herein. In some embodiments, PLP1 expression is reduced in at least one region of the brain and/or at least one region of the spinal cord for 1-6 months after administration of an oligonucleotide described herein. In some embodiments, PLP1 expression is reduced in at least one region of the brain and/or at least one region of the spinal cord for 1, 2, 3 or 4 months after administration of an oligonucleotide described herein. In some embodiments, PLP1 expression is reduced in at least one region of the brain and/or at least one region of the spinal cord for 1, 2, 3 4, 5 or 6 months after administration of an oligonucleotide described herein. In some embodiments, PLP1 expression is reduced in at least one region of the brain and/or at least one region of the spinal cord for 7-91 days after administration of an oligonucleotide described herein. In some embodiments, PLP1 expression is reduced in at least one region of the brain and/or at least one region of the spinal cord for 7, 14, 21, 28, 35, 42, 49, 56, 63, 70, 77, 84 or 91 days after administration of an oligonucleotide described herein.

Methods provided herein are useful in any appropriate cell type. In some embodiments, a cell is any cell that expresses PLP1 mRNA (e.g., oligodendrocyte). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides disclosed herein are delivered to a cell or population of cells using a nucleic acid delivery method known in the art including, but not limited to, injection of a solution or pharmaceutical composition containing the oligonucleotide, bombardment by particles covered by the oligonucleotide, exposing the cell or population of cells to a solution containing the oligonucleotide, or electroporation of cell membranes in the presence of the oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of PLP1 expression is determined by an assay or technique that evaluates one or more molecules, properties or characteristics of a cell or population of cells associated with PLP1 expression, or by an assay or technique that evaluates molecules that are directly indicative of PLP1 expression in a cell or population of cells (e.g., PLP1 mRNA or PLP1 protein). In some embodiments, the extent to which an oligonucleotide provided herein reduces PLP1 expression is evaluated by comparing PLP1 expression in a cell or population of cells contacted with the oligonucleotide to a control cell or population of cells (e.g., a cell or population of cells not contacted with the oligonucleotide or contacted with a control oligonucleotide). In some embodiments, a control amount or level of PLP1 expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide (e.g., a double-stranded oligonucleotide) described herein to a cell or a population of cells results in a reduction in PLP1 expression. In some embodiments, the reduction in PLP1 expression is relative to a control amount or level of PLP1 expression in cell or population of cells not contacted with the oligonucleotide or contacted with a control oligonucleotide. In some embodiments, the reduction in PLP1 expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of PLP1 expression. In some embodiments, the control amount or level of PLP1 expression is an amount or level of PLP1 mRNA and/or PLP1 protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, PLP1 expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about days, about 77 days, or about 84 days or more after contacting or delivering the oligonucleotide to the cell or population of cells. In some embodiments, PLP1 expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

### Reducing GFAP Expression

In some embodiments, the disclosure provides methods of reducing glial fibrillary acidic protein (GFAP) expression using an oligonucleotide described herein (i.e., a PLP1-targeting oligonucleotide). GFAP is a cytoskeletal protein found in mature astrocytes and is a marker for astrogliosis. Astrogliosis is a process generally defined by astrocytes responding to CNS damage and disease and encompasses molecular, cellular, and functional changes in astrocytes. Astrogliosis pathology ranges from mild to severe. Increased GFAP expression is observed for astrogliosis associated with, for example, Alzheimer's disease, Parkinson's disease, HIV-dementia and PMD.

In some embodiments, reducing GFAP expression comprises reducing an amount or level of GFAP mRNA, an amount or level of GFAP protein, or both. In some embodiments, GFAP expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks. In some embodiments, GFAP expression is reduced for about 1 month, 2 months, 3 months, 4 months, 5 months or 6 months. In some aspects, GFAP expression is reduced for about 7 days, 14 days, 21 days, 28 days, 35 days, 42 days, 49 days, 56 days, 63 days, 70 days, 77 days, 84 days, or 91 days.

In some embodiments, the disclosure provides methods for contacting or delivering to a cell or population of cells an effective amount of an oligonucleotide (e.g. PLP1-targeting dsRNAi oligonucleotide) herein to reduce GFAP expression. In some embodiments, a reduction of GFAP expression is determined by measuring a reduction in the amount or level of GFAP mRNA, GFAP protein, or GFAP activity in a cell. The methods include those described herein and known to one of ordinary skill in the art.

In some embodiments, the disclosure provides methods for reducing GFAP expression in the central nervous system (e.g., brain and spinal cord). In some embodiments, GFAP expression is reduced in at least one region of the brain. In some embodiments, GFAP expression is reduced in the frontal cortex, hippocampus, cerebellum, brain stem, lumbar spinal cord, or any combination thereof. In some embodiments, GFAP expression is reduced in at least one region of the spinal cord. In some embodiments, GFAP expression is reduced in the cervical spinal cord, thoracic spinal cord, lumbar spinal cord, lumbar dorsal root ganglion, or any combination thereof. In some embodiments, GFAP expression is reduced in at least one region of the brain and at least one region of the spinal cord. In some embodiments, GFAP expression is reduced in at least one of the lumbar spinal cord, frontal cortex, hippocampus, cerebellum, or brain stem.

In some embodiments, GFAP expression is reduced for 1-12 weeks after administration of an oligonucleotide described herein. In some embodiments, GFAP expression is reduced for 1, 2, 3, 4, 5, 6, 7, 8, 9, 10, 11 or 12 weeks after administration of an oligonucleotide described herein. In some embodiments, GFAP expression is reduced for 1-4 months after administration of an oligonucleotide described herein. In some embodiments, GFAP expression is reduced for 1-6 months after administration of an oligonucleotide described herein. In some embodiments, GFAP expression is reduced for 1, 2, 3 or 4 months after administration of an oligonucleotide described herein. In some embodiments, GFAP expression is reduced for 1, 2, 3 4, 5 or 6 months after administration of an oligonucleotide described herein. In some embodiments, GFAP expression is reduced for 7-91 days after administration of an oligonucleotide described herein. In some embodiments, GFAP expression is reduced for 7, 14, 21, 28, 35, 42, 49, 56, 63, 70, 77, or 84 or 91 days after administration of an oligonucleotide described herein.

In some embodiments, GFAP expression is reduced in at least one region of the brain and/or at least one region of the spinal cord for 1-12 weeks after administration of an oligonucleotide described herein. In some embodiments, GFAP expression is reduced in at least one region of the brain and/or at least one region of the spinal cord for 1, 2, 3, 4, 5, 6, 7, 8, 9, 10, 11 or 12 weeks after administration of an oligonucleotide described herein. In some embodiments, GFAP expression is reduced in at least one region of the brain and/or at least one region of the spinal cord for 1-4 months after administration of an oligonucleotide described herein. In some embodiments, GFAP expression is reduced in at least one region of the brain and/or at least one region of the spinal cord for 1-6 months after administration of an oligonucleotide described herein. In some embodiments, GFAP expression is reduced in at least one region of the brain and/or at least one region of the spinal cord for 1, 2, 3 or 4 months after administration of an oligonucleotide described herein. In some embodiments, GFAP expression is reduced in at least one region of the brain and/or at least one region of the spinal cord for 1, 2, 3 4, 5 or 6 months after administration of an oligonucleotide described herein. In some embodiments, GFAP expression is reduced in at least one region of the brain and/or at least one region of the spinal cord for 7-91 days after administration of an oligonucleotide described herein. In some embodiments, GFAP expression is reduced in at least one region of the brain and/or at least one region of the spinal cord for 7, 14, 21, 28, 35, 42, 49, 56, 63, 70, 77, 84 or 91 days after administration of an oligonucleotide described herein.

In some embodiments, contacting or delivering an oligonucleotide (e.g., a double-stranded oligonucleotide) described herein to a cell or a population of cells results in a reduction in GFAP expression. In some embodiments, the reduction in GFAP expression is relative to a control amount or level of GFAP expression in cell or population of cells not contacted with the oligonucleotide or contacted with a control oligonucleotide. In some embodiments, the reduction in GFAP expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, about 90% or lower, or about 99% or lower relative to a control amount or level of GFAP expression. In some embodiments, the control amount or level of GFAP expression is an amount or level of GFAP mRNA and/or GFAP protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, GFAP expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, GFAP expression is determined in a cell or population of cells at least about 1 month, about 2 months, about 3 months, about 4 months, about 5 months, or about 6 months or more after contacting or delivering the oligonucleotide to the cell or population of cells.

### Treatment Methods

The disclosure also provides oligonucleotides for use, or adaptable for use, to treat a subject (e.g., a human having a disease, disorder or condition associated with PLPJ expression) that would benefit from reducing PLP1 expression. In some aspects, the disclosure provides oligonucleotides for use, or adapted for use, to treat a subject having a disease, disorder or condition associated with expression of PLP1. The disclosure also provides oligonucleotides for use, or adaptable for use, in the manufacture of a medicament or pharmaceutical composition for treating a disease, disorder or condition associated with PLP1 expression. In some embodiments, the oligonucleotides for use, or adaptable for use, target PLP1 mRNA and reduce PLP1 expression (e.g., via the RNAi pathway). In some embodiments, the oligonucleotides for use, or adaptable for use, target PLP1 mRNA and reduce the amount or level of PLP1 mRNA, PLP1 protein and/or PLP1 activity.

In addition, in some embodiments of the methods herein, a subject having a disease, disorder or condition associated with PLP1 expression or is predisposed to the same is selected for treatment with an oligonucleotide (e.g., a double-stranded oligonucleotide) herein. In some embodiments, the method comprises selecting an individual having a marker (e.g., a biomarker) for a disease, disorder or condition associated with PLP1 expression, or predisposed to the same, such as, but not limited to, PLP1 mRNA, PLP1 protein, or a combination thereof. Likewise, and as detailed below, some embodiments of the methods provided by the disclosure include steps such as measuring or obtaining a baseline value for a marker of PLP1 expression (e.g., PLP1), and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with PLP1 expression with an oligonucleotide provided herein. In some aspects, the disclosure provides methods of treating or attenuating the onset or progression of a disease, disorder or condition associated with PLP1 expression using the oligonucleotides provided herein. In other aspects, the disclosure provides methods to achieve one or more therapeutic benefits in a subject having a disease, disorder or condition associated with PLP1 expression using the oligonucleotides provided herein. In some embodiments of the methods herein, the subject is treated by administering a therapeutically effective amount of any one or more of the oligonucleotides provided herein. In some embodiments, treatment comprises reducing PLP1 expression. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with PLP1 expression such that PLP1 expression is reduced in the subject, thereby treating the subject. In some embodiments, an amount or level of PLP1 mRNA is reduced in the subject. In some embodiments, an amount or level of PLP1 protein is reduced in the subject.

In some embodiments of the methods herein, an oligonucleotide provided herein, or a pharmaceutical composition comprising the oligonucleotide, is administered to a subject having a disease, disorder or condition associated with PLP1 expression such that PLP1 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to PLP1 expression prior to administration of the oligonucleotide or pharmaceutical composition. In some embodiments of the methods herein, an oligonucleotide provided herein, or a pharmaceutical composition comprising the oligonucleotide, is administered to a subject having a disease, disorder or condition associated with PLP1 expression such that PLP1 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to PLP1 expression prior to administration of the oligonucleotide or pharmaceutical composition. In some embodiments, PLP1 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to PLP1 expression in a subject (e.g., a reference or control subject) not receiving the oligonucleotide or pharmaceutical composition or receiving a control oligonucleotide, pharmaceutical composition or treatment. In some embodiments, PLP1 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to PLP1 expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with PLP1 expression such that an amount or level of PLP1 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of PLP1 mRNA prior to administration of the oligonucleotide or pharmaceutical composition. In some embodiments of the methods herein, an oligonucleotide herein, or a pharmaceutical composition comprising the oligonucleotide, is administered to a subject having a disease, disorder or condition associated with PLP1 expression such that an amount or level of PLP1 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to the amount or level of PLP1 mRNA prior to administration of the oligonucleotide or pharmaceutical composition. In some embodiments, an amount or level of PLP1 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of PLP1 mRNA in a subject (e.g., a reference or control subject) not receiving the oligonucleotide or pharmaceutical composition or receiving a control oligonucleotide, pharmaceutical composition or treatment. In some embodiments, an amount or level of PLP1 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to an amount or level of PLP1 mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with PLP1 expression such that an amount or level of PLP1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of PLP1 protein prior to administration of the oligonucleotide or pharmaceutical composition. In some embodiments of the methods herein, an oligonucleotide herein, or a pharmaceutical composition comprising the oligonucleotide, is administered to a subject having a disease, disorder or condition associated with PLP1 expression such that an amount or level of PLP1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to the amount or level of PLP1 protein prior to administration of the oligonucleotide or pharmaceutical composition. In some embodiments, an amount or level of PLP1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of PLP1 protein in a subject (e.g., a reference or control subject) not receiving the oligonucleotide or pharmaceutical composition or receiving a control oligonucleotide, pharmaceutical composition or treatment. In some embodiments, an amount or level of PLP1 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to an amount or level of PLP1 protein in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with PLP1 expression such that an amount or level of PLP1 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of PLP1 activity prior to administration of the oligonucleotide or pharmaceutical composition. In some embodiments of the methods herein, an oligonucleotide herein, or a pharmaceutical composition comprising the oligonucleotide, is administered to a subject having a disease, disorder or condition associated with PLP1 expression such that an amount or level of PLP1 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to the amount or level of PLP1 activity prior to administration of the oligonucleotide or pharmaceutical composition. In some embodiments, an amount or level of PLP1 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of PLP1 activity in a subject (e.g., a reference or control subject) not receiving the oligonucleotide or pharmaceutical composition or receiving a control oligonucleotide, pharmaceutical composition or treatment. In some embodiments, an amount or level of PLP1 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% for 1-12 weeks, 1-6 months, or 7-91 days when compared to an amount or level of PLP1 activity in a subject (e.g., a reference or control subject) not receiving the oligonucleotide or pharmaceutical composition or receiving a control oligonucleotide, pharmaceutical composition or treatment.

Suitable methods for determining PLP1 expression, an amount or level of PLP1 mRNA, an amount or level of PLP1 protein, and/or an amount or level of PLP1 activity, in the subject, or in a sample from the subject, are known in the art. Further, the Examples set forth herein illustrate exemplary methods for determining PLP1 expression.

In some embodiments, PLP1 expression, an amount or level of PLP1 mRNA, an amount or level of PLP1 protein, an amount or level of PLP1 activity, or any combination thereof, is reduced in a cell (e.g., an oligodendrocyte), a population or a group of cells (e.g., an organoid), an organ (e.g., frontal cortex), blood or a fraction thereof (e.g., plasma), a tissue (e.g., brain tissue), a sample (e.g., a brain biopsy sample), or any other biological material obtained or isolated from the subject. In some embodiments, PLP1 expression, an amount or level of PLP1 mRNA, an amount or level of PLP1 protein, an amount or level of PLP1 activity, or any combination thereof, is reduced in more than one type of cell (e.g., an oligodendrocyte and one or more other type(s) of cell), more than one groups of cells, more than one organ (e.g., brain and one or more other organ(s)), more than one fraction of blood (e.g., plasma and one or more other blood fraction(s)), more than one type of tissue (e.g., brain tissue and one or more other type(s) of tissue), more than one type of sample (e.g., a brain biopsy sample and one or more other type(s) of biopsy sample) obtained or isolated from the subject. In some embodiments, PLP1 expression, an amount or level of PLP1 mRNA, an amount or level of PLP1 protein, an amount or level of PLP1 activity, or any combination thereof is reduced in one or more of the frontal cortex, parietal cortex, temporal cortex, occipital cortex, cerebellum, brainstem, cervical spinal cord, thoracic spinal cord, lumbar spinal cord, or lumbar dorsal root ganglion.

Examples of a disease, disorder or condition associated with PLP1 expression include, Pelizaeus-Merzbacher disease (PMD) and spastic paraplegia 2 (SPG2). In a disease, disorder or condition associated with PLP1 expression (e.g., PMD, SPG2), PLP1 expression is aberrant and results in pathology. For examples, most mutations in the PLP1 gene that cause Pelizaeus-Merzbacher disease result in a duplication of the PLP1 gene. PLP1 gene duplication in PMD results in increased production of proteolipid protein 1 and DM20. Other mutations lead to production of abnormal proteins that are often misfolded. Excess or abnormal proteins become trapped within cell structures and cannot travel to the cell membrane. As a result, proteolipid protein 1 and DM20 are not available to form myelin. The accumulation of excess proteins leads to swelling and breakdown of nerve fibers. Other mutations delete the PLP1 gene, which prevents proteolipid protein 1 and DM20 protein production and results in a lack of these proteins in the cell membrane, which causes any myelin that is formed to be unstable and quickly broken down. All of these PLP1 gene mutations lead to hypomyelination, nerve fiber damage, and impairment of nervous system function, resulting in the signs and symptoms of Pelizaeus-Merzbacher disease (Garbern (2007) MOL LIFE SCI 64(1):50-65; Garbern (2005) J NEUROL SCI 228(2):201-03).

In some embodiments, an oligonucleotide provided herein, or a pharmaceutical composition comprising the oligonucleotide, is administered to a subject having a disease, disorder or condition associated with PLP1 expression such that GFAP expression is reduced in the subject, thereby treating the subject. In some embodiments, an oligonucleotide provided herein, or a pharmaceutical composition comprising the oligonucleotide, is administered to a subject having a disease, disorder or condition associated with PLP1 expression such that GFAP expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to GFAP expression prior to administration of the oligonucleotide or pharmaceutical composition.

In some embodiments, the disclosure provides methods of reducing astrogliosis associated with PLP1 expression in a subject. In some embodiments, astrogliosis is measured by, but not limited to, up-regulation of GFAP, cellular hypertrophy, and astrocyte proliferation. Methods of measuring GFAP expression, cellular hypertrophy, and astrocyte proliferation are known in the art. Examples include, but are not limited to immunostaining, qPCR, and western blot analysis. In some embodiments, an oligonucleotide herein, or a pharmaceutical composition comprising the oligonucleotide, is administered to a subject having astrogliosis associated with PLP1 expression such that astrogliosis is reduced as measured by reduced GFAP expression. In some embodiments, an oligonucleotide herein, or a pharmaceutical composition comprising the oligonucleotide, is administered to a subject having astrogliosis associated with PLP1 expression such that astrogliosis is reduced as measured by reduced cellular hypertrophy. In some embodiments, an oligonucleotide herein, or a pharmaceutical composition comprising the oligonucleotide, is administered to a subject having astrogliosis associated with PLP1 expression such that astrogliosis is reduced as measured by reduced cellular proliferation.

In some embodiments, the disclosure provides methods of reducing demyelination in a subject with a disease, disorder or condition associated with PLP1 expression. Demyelination is loss of myelin, a type of fatty tissue that surrounds and protects nerves throughout the body. Demyelination causes neurological deficits, such as vision changes, weakness, altered sensation and behavioral or cognitive problems. Methods for measuring demyelination are known to those of skill in the art and include, but are not limited to, measuring the level of biomarkers associated with demyelination such as MBP (myelin basic protein), and imaging of brains to identify demyelination. In some embodiments, methods for reducing demyelination comprise administering an RNAi oligonucleotide described herein to a subject in need thereof.

Because of their high specificity, the oligonucleotides herein (e.g., dsRNAi oligonucleotides) specifically target mRNAs of target genes of cells, tissue(s), or organ(s) (e.g., brain). In preventing disease, the target gene may be one which is required for initiation or maintenance of the disease or which has been identified as being associated with a higher risk of contracting the disease. In treating disease, the oligonucleotide can be brought into contact with the cells, tissue(s), or organ(s) (e.g., brain) exhibiting or responsible for mediating the disease. For example, an oligonucleotide substantially identical to all or part of a wild-type (i.e., native) or mutated gene associated with a disorder or condition associated with PLP1 expression may be brought into contact with or introduced into a cell or tissue type of interest such as an oligodendrocyte or other brain cell.

In some embodiments, the target gene may be a target gene from any mammal, such as a human. Any gene may be silenced according to the method described herein.

Methods described herein are typically involve administering to a subject a therapeutically effective amount of an oligonucleotide herein (e.g., a dsRNAi oligonucleotide), that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the brain of a subject). Typically, oligonucleotides herein (e.g., dsRNAi oligonucleotides) are administered intravenously or subcutaneously. In some embodiments, the oligonucleotides described herein are administered to the cerebral spinal fluid. In some embodiments, the oligonucleotides described herein are administered intrathecally. In some embodiments, the oligonucleotides described herein are administered intracerebroventricularly. In some embodiments, the oligonucleotides described herein are administered by intracisternal magna injection.

As a non-limiting set of examples, the oligonucleotides herein (e.g., dsRNAi oligonucleotides) would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Methods for Determining Responsiveness to Treatment

In some embodiments, a disease, disorder or condition associated with PLP1 expression is also associated with increased GFAP expression. Accordingly, in some embodiments, GFAP expression (protein or mRNA) is a biomarker for determining the response to treatment of a PLP1 targeting RNAi oligonucleotide in a patient. In some embodiments, GFAP expression is a biomarker for monitoring response to treatment with a PLP1 targeting RNAi oligonucleotide in a patient.

In some embodiments, the disclosure provides methods for determining treatment response in a patient that has received or is receiving an RNAi oligonucleotide treatment targeting PLP1, the method comprising determining a level of GFAP expression in the patient, wherein a reduction in the level of GFAP expression indicates response to treatment.

In some embodiments, the disclosure provides methods for determining treatment response in a patient with a disease, disorder or condition associated with PLP1 expression, the method comprising (i) administering an RNAi oligonucleotide treatment targeting PLP1, and (ii) determining a level of GFAP expression in the patient, wherein a reduction in the level of GFAP expression indicates response to treatment. In some embodiments, the disclosure provides methods for determining treatment response in a patient with a disease, disorder or condition associated with PLP1 expression, the method comprising (i) administering an RNAi oligonucleotide treatment targeting PLP1, wherein the RNAi oligonucleotide comprises a sense strand comprising the nucleotide sequence of SEQ ID NO: 76 and antisense strand comprising the nucleotide sequence of SEQ ID NO: 77, and (ii) determining a level of GFAP expression in the patient, wherein a reduction in the level of GFAP expression indicates response to treatment. In some embodiments, the disclosure provides methods for determining treatment response in a patient with a disease, disorder or condition associated with PLP1 expression, the method comprising (i) administering an RNAi oligonucleotide treatment targeting PLP1, wherein the RNAi oligonucleotide comprises a sense strand comprising the nucleotide sequence of SEQ ID NO: 112 and antisense strand comprising the nucleotide sequence of SEQ ID NO: 113, and (ii) determining a level of GFAP expression in the patient, wherein a reduction in the level of GFAP expression indicates response to treatment. In some embodiments, the disclosure provides methods for determining treatment response in a patient with a disease, disorder or condition associated with PLP1 expression, the method comprising (i) administering an RNAi oligonucleotide treatment targeting PLP1, wherein the RNAi oligonucleotide comprises a sense strand comprising the nucleotide sequence of SEQ ID NO: 191 and antisense strand comprising the nucleotide sequence of SEQ ID NO: 192, and (ii) determining a level of GFAP expression in the patient, wherein a reduction in the level of GFAP expression indicates response to treatment. In some embodiments, the disclosure provides methods for determining treatment response in a patient with a disease, disorder or condition associated with PLP1 expression, the method comprising (i) administering an RNAi oligonucleotide treatment targeting PLP1, wherein the RNAi oligonucleotide comprises a sense strand comprising the nucleotide sequence of SEQ ID NO: 191 and antisense strand comprising the nucleotide sequence of SEQ ID NO: 207, and (ii) determining a level of GFAP expression in the patient, wherein a reduction in the level of GFAP expression indicates response to treatment.

In some embodiments, the disclosure provides methods for determining treatment response in a patient with astrogliosis, the method comprising (i) administering an RNAi oligonucleotide treatment targeting PLP1, and (ii) determining a level of GFAP expression in the patient, wherein a reduction in the level of GFAP expression indicates response to treatment. In some embodiments, the disclosure provides methods for determining treatment response in a patient with astrogliosis, the method comprising (i) administering an RNAi oligonucleotide treatment targeting PLP1, wherein the RNAi oligonucleotide comprises a sense strand comprising the nucleotide sequence of SEQ ID NO: 76 and antisense strand comprising the nucleotide sequence of SEQ ID NO: 77, and (ii) determining a level of GFAP expression in the patient, wherein a reduction in the level of GFAP expression indicates response to treatment. In some embodiments, the disclosure provides methods for determining treatment response in a patient with astrogliosis, the method comprising (i) administering an RNAi oligonucleotide treatment targeting PLP1, wherein the RNAi oligonucleotide comprises a sense strand comprising the nucleotide sequence of SEQ ID NO: 112 and antisense strand comprising the nucleotide sequence of SEQ ID NO: 113, and (ii) determining a level of GFAP expression in the patient, wherein a reduction in the level of GFAP expression indicates response to treatment. In some embodiments, the disclosure provides methods for determining treatment response in a patient with astrogliosis, the method comprising (i) administering an RNAi oligonucleotide treatment targeting PLP1, wherein the RNAi oligonucleotide comprises a sense strand comprising the nucleotide sequence of SEQ ID NO: 191 and antisense strand comprising the nucleotide sequence of SEQ ID NO: 192, and (ii) determining a level of GFAP expression in the patient, wherein a reduction in the level of GFAP expression indicates response to treatment. In some embodiments, the disclosure provides methods for determining treatment response in a patient with astrogliosis, the method comprising (i) administering an RNAi oligonucleotide treatment targeting PLP1, wherein the RNAi oligonucleotide comprises a sense strand comprising the nucleotide sequence of SEQ ID NO: 191 and antisense strand comprising the nucleotide sequence of SEQ ID NO: 207, and (ii) determining a level of GFAP expression in the patient, wherein a reduction in the level of GFAP expression indicates response to treatment.

In some embodiments, the level of GFAP expression is compared to a pre-determined healthy range of GFAP expression. In some embodiments, the pre-determined healthy range is based on GFAP expression in a population of patients that were not experiencing brain damage or brain injury at the time of measuring GFAP expression. In some embodiments, the level of GFAP expression is compared to a pre-determined diseased range of GFAP expression. In some embodiments, the pre-determined diseased range of GFAP expression is based on GFAP expression in a population of patients that had brain damage or brain injury at the time of measuring GFAP expression. In some embodiments, the pre-determined diseased range of GFAP expression is based on GFAP expression in a population of patients that had astrogliosis at the time of measuring GFAP expression.

In some embodiments, after a patient is administered an RNAi oligonucleotide treatment targeting PLP1, the level of GFAP expression is reduced from a pre-determined diseased range to a pre-determined healthy range.

In some embodiments, a level of GFAP expression is determined before the patient receives a dose of the RNAi oligonucleotide treatment targeting PLP1. In some embodiments, a level of GFAP expression is determined before the patient receives an initial dose of the RNAi oligonucleotide treatment targeting PLP1 and throughout the course of treatment.

GFAP has previously been identified as a circulating biomarker of neuronal and glial injury. Accordingly, in some embodiments, a patient with a disease, disorder or condition associated with PLP1 has GFAP expressed in the circulatory system. In some embodiments, GFAP expression is determined in a sample from the patient. In some embodiments, the sample is selected from blood, serum, plasma and cerebral spinal fluid.

In some embodiments, the RNAi oligonucleotide treatment targeting PLP1 is any of the oligonucleotides described herein.

Methods for determining GFAP expression in a sample from a patient are known to those of skill in the art. Exemplary methods include, but are not limited to, immunoassay, an immunoblotting method, an immunoprecipitation assay, an immunostaining method, a quantitative assay, an immunofluorescent assay, or a chemiluminescence assay. In some embodiments, the GFAP level is measured by an immunoassay, for example, an enzyme linked immunosorbent assay (ELISA) using an antibody or an antigen binding fragment thereof that specifically binds GFAP.

### Kits

In some embodiments, the disclosure provides a kit comprising an oligonucleotide described herein, and instructions for use. In some embodiments, the kit comprises an oligonucleotide described herein, and a package insert containing instructions for use of the kit and/or any component thereof. In some embodiments, the kit comprises, in a suitable container, an oligonucleotide described herein, one or more controls, and various buffers, reagents, enzymes and other standard ingredients well known in the art. In some embodiments, the container comprises at least one vial, well, test tube, flask, bottle, syringe or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide described herein, and a pharmaceutically acceptable carrier, or a pharmaceutical composition comprising the oligonucleotide and instructions for treating or delaying progression of a disease, disorder or condition associated with PLP1 expression in a subject in need thereof.

In some embodiments, a kit comprises an oligonucleotide described herein and a pharmaceutically acceptable carrier or a pharmaceutical composition comprising the oligonucleotide, and instructions for administering the oligonucleotide or pharmaceutical composition to the cerebral spinal fluid to reduce PLP1 expression in at least one region of the brain and/or at least one region of the spinal cord in a subject in need thereof.

### Other Embodiments

The disclosure relates to the following embodiments. Throughout this section, the term embodiment is abbreviated as “E” followed by an ordinal. For example, E1 is equivalent to Embodiment 1.

E1. An RNAi oligonucleotide for reducing PLP1 expression, the oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

E2. The RNAi oligonucleotide of embodiment 1, wherein the sense strand is 15 to 50 nucleotides in length.

E3. The RNAi oligonucleotide of embodiments 1 or 2, wherein the sense strand is 18 to 36 nucleotides in length.

E4. The RNAi oligonucleotide of any one of embodiments 1 to 3, wherein the antisense strand is 15 to 30 nucleotides in length.

E5. The RNAi oligonucleotide of any one of embodiments 1 to 4, wherein the antisense strand is 22 nucleotides in length and wherein antisense strand and the sense strand form a duplex region of at least 19 nucleotides in length, optionally at least 20 nucleotides in length.

E6. The RNAi oligonucleotide of any one of embodiments 1 to 5, wherein the region of complementarity is at least 19 contiguous nucleotides in length, optionally at least 20 nucleotides in length.

E7. The RNAi oligonucleotide of any one of embodiments 1 to 6, wherein the 3′ end of the sense strand comprises a stem-loop set forth as S1-L-S2, wherein S1 is complementary to S2, and wherein L forms a loop between S1 and S2 of 3-5 nucleotides in length.

E8. An RNAi oligonucleotide for reducing PLP1 expression, the oligonucleotide comprising a sense strand of 15 to 50 nucleotides in length and an antisense strand, wherein the sense strand and the antisense strand form a duplex region, wherein the wherein the antisense strand comprises a region of complementarity to a PLP1 mRNA target sequence of any one of SEQ ID NOs: 171-188, and wherein the region of complementarity is at least 15 contiguous nucleotides in length.

E9. An RNAi oligonucleotide for reducing PLP1 expression, the oligonucleotide comprising a sense strand of 15 to 50 nucleotides in length and an antisense strand of 15 to 30 nucleotides in length, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

E10. An RNAi oligonucleotide for reducing PLP1 expression, the oligonucleotide comprising a sense strand of 15 to 50 nucleotides in length and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

E11. An RNAi oligonucleotide for reducing PLP1 expression, the oligonucleotide comprising a sense strand of 18 to 36 nucleotides in length and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally nucleotides in length.

E12. An RNAi oligonucleotide for reducing PLP1 expression, the oligonucleotide comprising a sense strand of 18 to 36 nucleotides in length and an antisense strand of 22 nucleotides in length, wherein the sense strand and the antisense strand form a duplex region, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

E13. An RNAi oligonucleotide for reducing PLP1 expression, the oligonucleotide comprising a sense strand of 18 to 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

E14. An RNAi oligonucleotide for reducing PLP1 expression, the oligonucleotide comprising a sense strand of 36 nucleotides in length and an antisense strand of 22 nucleotides in length, wherein the sense strand and the antisense strand form a duplex region, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

E15. An RNAi oligonucleotide for reducing PLP1 expression, the oligonucleotide comprising a sense strand of 36 nucleotides in length and an antisense strand of 22 nucleotides in length, wherein the sense strand and the antisense strand form a duplex region of at least 19 nucleotides in length, optionally 20 nucleotides in length, wherein the 3′ end of the sense strand comprises a stem-loop set forth as S1-L-S2, wherein S1 is complementary to S2, and wherein L forms a loop between S1 and S2 of 3-5 nucleotides in length, wherein the antisense strand comprises a region of complementarity to a PLP1 mRNA target sequence of any one of SEQ ID NOs: 171-188, and wherein the region of complementarity is 19 contiguous nucleotides in length, optionally 20 nucleotides in length.

E16. The RNAi oligonucleotide of any one of embodiments 1-15, wherein the region of complementarity differs by no more than 3 nucleotides in length to the PLP1 mRNA target sequence.

E17. The RNAi oligonucleotide of any one of embodiments 1-15, wherein the region of complementarity is fully complementary to the PLP1 mRNA target sequence.

E18. The RNAi oligonucleotide of any one of embodiments 7 and 13-17, wherein L is a triloop or a tetraloop.

E19. The RNAi oligonucleotide of embodiment 18, wherein L is a tetraloop.

E20. The RNAi oligonucleotide of embodiment 19, wherein the tetraloop comprises the sequence 5′-GAAA-3′.

E21. The RNAi oligonucleotide of any one of embodiments 7 and 13-20, wherein one or more of the nucleotides of L comprise a 2′-O-methyl modification.

E22. The RNAi oligonucleotide of embodiment 21, wherein each nucleotide of L comprises a 2′-O-methyl modification.

E23. The RNAi oligonucleotide of any one of embodiments 7 and 13-22, wherein the S1 and S2 are 1-10 nucleotides in length and have the same length.

E24. The RNAi oligonucleotide of embodiment 23, wherein S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length.

E25. The RNAi oligonucleotide of embodiment 24, wherein S1 and S2 are 6 nucleotides in length.

E26. The RNAi oligonucleotide of any one of embodiments 7 and 13 to 25, wherein the stem-loop comprises the sequence 5′-GCAGCCGAAAGGCUGC-3′ (SEQ ID NO: 190).

E27. The RNAi oligonucleotide of any one of embodiments 1 to 26, wherein the antisense strand comprises a 3′ overhang sequence of one or more nucleotides in length.

E28. The RNAi oligonucleotide of embodiment 27, wherein the 3′ overhang sequence is 2 nucleotides in length, optionally wherein the 3′ overhang sequence is GG.

E29. The RNAi oligonucleotide of any one of the preceding embodiments, wherein the oligonucleotide comprises at least one modified nucleotide.

E30. The RNAi oligonucleotide of embodiment 29, wherein the modified nucleotide comprises a 2′-modification.

E31. The RNAi oligonucleotide of embodiment 30, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.

E32. The RNAi oligonucleotide of any one of embodiments 29 to 31, wherein all nucleotides comprising the oligonucleotide are modified, optionally wherein the modification is a 2′-modification selected from 2′-fluoro and 2′-O-methyl.

E33. The RNAi oligonucleotide of embodiment 29, wherein about 10-15%, 10%, 11%, 12%, 13%, 14% or 15% of the nucleotides of the sense strand comprise a 2′-fluoro modification.

E34. The RNAi oligonucleotide of embodiment 29 or 33, wherein about 25-35%, 25%, 26%, 27%, 28%, 29%, 30%, 31%, 32%, 33%, 34% or 35% of the nucleotides of the antisense strand comprise a 2′-fluoro modification.

E35. The RNAi oligonucleotide of embodiment 29, wherein about 15-25%, 15%, 16%, 17%, 18%, 19%, 20%, 21%, 22%, 23%, 24%, or 25% of the nucleotides of the oligonucleotide comprise a 2′-fluoro modification.

E36. The RNAi oligonucleotide of embodiment 29, wherein the sense strand comprises 36 nucleotides with positions numbered 1-36 from 5′ to 3′, wherein positions 8-11 comprise a 2′-fluoro modification.

E37. The RNAi oligonucleotide of embodiment 29 or 36, wherein the antisense strand comprises 22 nucleotides with positions numbered 1-22 from 5′ to 3′, and wherein positions 2, 3, 4, 5, 7, 10 and 14 comprise a 2′-fluoro modification.

E38. The RNAi oligonucleotide of any one of embodiments 33-37, wherein the remaining nucleotides of the oligonucleotide comprise a 2′-O-methyl modification.

E39. The RNAi oligonucleotide of any one of the preceding embodiments, wherein the oligonucleotide comprises at least one modified internucleotide linkage.

E40. The RNAi oligonucleotide of embodiment 39, wherein the at least one modified internucleotide linkage is a phosphorothioate linkage.

E41. The RNAi oligonucleotide of any one of the preceding embodiments, wherein the 4′-carbon of the sugar of the 5′-nucleotide of the antisense strand comprises a phosphate analog.

E42. The RNAi oligonucleotide of embodiment 41, wherein the phosphate analog is oxymethylphosphonate, vinylphosphonate or malonylphosphonate, optionally wherein the phosphate analog is a 4′-phosphate analog comprising 5′-methoxyphosphonate-4′-oxy.

E43. The RNAi oligonucleotide of any one of the preceding embodiments, wherein at least one nucleotide of the oligonucleotide is conjugated to one or more targeting ligands.

E44. The RNAi oligonucleotide of embodiment 43, wherein each targeting ligand comprises a carbohydrate, amino sugar, cholesterol, polypeptide or lipid.

E45. The RNAi oligonucleotide of embodiment 43, wherein each targeting ligand comprises a N-acetylgalactosamine (GalNAc) moiety.

E46. The RNAi oligonucleotide of embodiment 45, wherein the GalNac moiety is a monovalent GalNAc moiety, a bivalent GalNAc moiety, a trivalent GalNAc moiety or a tetravalent GalNAc moiety.

E47. The RNAi oligonucleotide of any one of embodiments 13 to 46, wherein up to 4 nucleotides of L of the stem-loop are each conjugated to a monovalent GalNAc moiety.

E48. The RNAi oligonucleotide of any one of embodiments 1 to 47, wherein the sense strand comprises a nucleotide sequence of any one of SEQ ID NOs: 76, 78, 80, 82, 84, 86, 88, 90, 92, 94, 96, 98, 100, 102, 104, 106, 108, and 110.

E49. The RNAi oligonucleotide of any one of embodiments 1 to 48, wherein the antisense strand comprises a nucleotide sequence of any one of SEQ ID NOs: 77, 79, 81, 83, 85, 87, 89, 91, 93, 95, 97, 99, 101, 103, 105, 107, 109, and 111.

E50. The RNAi oligonucleotide of any one of embodiments 1 to 49, wherein the sense strand and antisense strands comprise nucleotide sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(l) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

E51. The RNAi oligonucleotide of any one of embodiments 1 to 49, wherein the sense strand comprises a nucleotide sequence as set forth in SEQ ID NO: 76, wherein the antisense strand comprises a nucleotide sequence as set forth in SEQ ID NO: 77.

E52. The RNAi oligonucleotide of any one of embodiments 1 to 49, wherein the sense strand comprises a nucleotide sequence as set forth in SEQ ID NO: 78, wherein the antisense strand comprises a nucleotide sequence as set forth in SEQ ID NO: 79.

E53. The RNAi oligonucleotide of any one of embodiments 1 to 49, wherein the sense strand comprises a nucleotide sequence as set forth in SEQ ID NO: 80, wherein the antisense strand comprises a nucleotide sequence as set forth in SEQ ID NO: 81.

E54. The RNAi oligonucleotide of any one of embodiments 1 to 49, wherein the sense strand comprises a nucleotide sequence as set forth in SEQ ID NO: 82, wherein the antisense strand comprises a nucleotide sequence as set forth in SEQ ID NO: 83.

E55. The RNAi oligonucleotide of any one of embodiments 1 to 49, wherein the sense strand comprises a nucleotide sequence as set forth in SEQ ID NO: 84, wherein the antisense strand comprises a nucleotide sequence as set forth in SEQ ID NO: 85.

E56. The RNAi oligonucleotide of any one of embodiments 1 to 49, wherein the sense strand comprises a nucleotide sequence as set forth in SEQ ID NO: 86, wherein the antisense strand comprises a nucleotide sequence as set forth in SEQ ID NO: 87.

E57. The RNAi oligonucleotide of any one of embodiments 1 to 49, wherein the sense strand comprises a nucleotide sequence as set forth in SEQ ID NO: 88, wherein the antisense strand comprises a nucleotide sequence as set forth in SEQ ID NO: 89.

E58. The RNAi oligonucleotide of any one of embodiments 1 to 49, wherein the sense strand comprises a nucleotide sequence as set forth in SEQ ID NO: 90, wherein the antisense strand comprises a nucleotide sequence as set forth in SEQ ID NO: 91.

E59. The RNAi oligonucleotide of any one of embodiments 1 to 49, wherein the sense strand comprises a nucleotide sequence as set forth in SEQ ID NO: 92, wherein the antisense strand comprises a nucleotide sequence as set forth in SEQ ID NO: 93.

E60. The RNAi oligonucleotide of any one of embodiments 1 to 49, wherein the sense strand comprises a nucleotide sequence as set forth in SEQ ID NO: 94, wherein the antisense strand comprises a nucleotide sequence as set forth in SEQ ID NO: 95.

E61. The RNAi oligonucleotide of any one of embodiments 1 to 49, wherein the sense strand comprises a nucleotide sequence as set forth in SEQ ID NO: 96, wherein the antisense strand comprises a nucleotide sequence as set forth in SEQ ID NO: 97.

E62. The RNAi oligonucleotide of any one of embodiments 1 to 49, wherein the sense strand comprises a nucleotide sequence as set forth in SEQ ID NO: 98, wherein the antisense strand comprises a nucleotide sequence as set forth in SEQ ID NO: 99.

E63. The RNAi oligonucleotide of any one of embodiments 1 to 49, wherein the sense strand comprises a nucleotide sequence as set forth in SEQ ID NO: 100, wherein the antisense strand comprises a nucleotide sequence as set forth in SEQ ID NO: 101.

E64. The RNAi oligonucleotide of any one of embodiments 1 to 49, wherein the sense strand comprises a nucleotide sequence as set forth in SEQ ID NO: 102, wherein the antisense strand comprises a nucleotide sequence as set forth in SEQ ID NO: 103.

E65. The RNAi oligonucleotide of any one of embodiments 1 to 49, wherein the sense strand comprises a nucleotide sequence as set forth in SEQ ID NO: 104, wherein the antisense strand comprises a nucleotide sequence as set forth in SEQ ID NO: 105.

E66. The RNAi oligonucleotide of any one of embodiments 1 to 49, wherein the sense strand comprises a nucleotide sequence as set forth in SEQ ID NO: 106, wherein the antisense strand comprises a nucleotide sequence as set forth in SEQ ID NO: 107.

E67. The RNAi oligonucleotide of any one of embodiments 1 to 49, wherein the sense strand comprises a nucleotide sequence as set forth in SEQ ID NO: 108, wherein the antisense strand comprises a nucleotide sequence as set forth in SEQ ID NO: 109.

E68. The RNAi oligonucleotide of any one of embodiments 1 to 49, wherein the sense strand comprises a nucleotide sequence as set forth in SEQ ID NO: 110, wherein the antisense strand comprises a nucleotide sequence as set forth in SEQ ID NO: 111.

E69. An RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the antisense strand comprises a region of complementarity to a PLP1 mRNA target sequence of any one of SEQ ID NOs: 171-188, and wherein the region of complementarity is at least 15 contiguous nucleotides in length.

E70. An RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

E71. An RNAi oligonucleotide for reducing PLP1 expression, the oligonucleotide comprising a sense strand and an antisense strand, wherein the sense strand and the antisense strand form a duplex region, wherein all nucleotides comprising the sense strand and antisense strand are modified, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

E72. An RNAi oligonucleotide for reducing PLP1 expression, the oligonucleotide comprising a sense strand and an antisense strand, wherein the sense strand and the antisense strand form a duplex region, wherein all nucleotides comprising the sense strand and the antisense strand are modified, wherein the antisense strand and the sense strand comprise one or more 2′-fluoro and 2′-O-methyl modified nucleotides and at least one phosphorothioate linkage, wherein the 4′-carbon of the sugar of the 5′-nucleotide of the antisense strand comprises a phosphate analog, wherein the antisense strand comprises a region of complementarity to a PLP1 mRNA target sequence of any one of SEQ ID NOs: 171-188, and wherein the region of complementarity is at least 15 contiguous nucleotides in length.

E73. The RNAi oligonucleotide of any one of embodiments 69-72, wherein the region of complementarity differs by no more than 3 nucleotides in length to the PLP1 mRNA target sequence.

E74. The RNAi oligonucleotide of any one of embodiments 69-72, wherein the region of complementarity is fully complementary to the PLP1 mRNA target sequence.

E75. The RNAi oligonucleotide of any one of embodiments 69 to 72, wherein the sense strand comprises of any one of SEQ ID NOs: 112, 114, 116, 118, 120, 122, 124, 126, 128, 130, 132, 134, 136, 138, 140, 142, 144, 146, and 191.

E76. The RNAi oligonucleotide of any one of embodiments 69 to 75, wherein the antisense strand comprises of any one of SEQ ID NOs: 113, 115, 117, 119, 121, 123, 125, 127, 129, 131, 133, 135, 137, 139, 141, 143, 145, 147, and 192.

E77. The RNAi oligonucleotide of any one of embodiments 69 to 76, wherein the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(l) SEQ ID NOs: 122 and 123, respectively;
(g) SEQ ID NOs: 124 and 125, respectively;
(h) SEQ ID NOs: 126 and 127, respectively
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 192, respectively.

E78. The RNAi oligonucleotide of any one of embodiments 69 to 77, wherein the sense strand comprises SEQ ID NO: 112, and wherein the antisense strand comprises SEQ ID NO: 113.

E79. The RNAi oligonucleotide of any one of embodiments 69 to 77, wherein the sense strand comprises SEQ ID NO: 114, and wherein the antisense strand comprises SEQ ID NO: 115.

E80. The RNAi oligonucleotide of any one of embodiments 69 to 77, wherein the sense strand comprises SEQ ID NO: 116, and wherein the antisense strand comprises SEQ ID NO: 117.

E81. The RNAi oligonucleotide of any one of embodiments 69 to 77, wherein the sense strand comprises SEQ ID NO: 118, and wherein the antisense strand comprises SEQ ID NO: 119.

E82. The RNAi oligonucleotide of any one of embodiments 69 to 77, wherein the sense strand comprises SEQ ID NO: 120, and wherein the antisense strand comprises SEQ ID NO: 121.

E83. The RNAi oligonucleotide of any one of embodiments 69 to 77, wherein the sense strand comprises SEQ ID NO: 122, and wherein the antisense strand comprises SEQ ID NO: 123.

E84. The RNAi oligonucleotide of any one of embodiments 69 to 77, wherein the sense strand comprises SEQ ID NO: 124, and wherein the antisense strand comprises SEQ ID NO: 125.

E85. The RNAi oligonucleotide of any one of embodiments 69 to 77, wherein the sense strand comprises SEQ ID NO: 126, and wherein the antisense strand comprises SEQ ID NO: 127.

E86. The RNAi oligonucleotide of any one of embodiments 69 to 77, wherein the sense strand comprises SEQ ID NO: 128, and wherein the antisense strand comprises SEQ ID NO: 129.

E87. The RNAi oligonucleotide of any one of embodiments 69 to 77, wherein the sense strand comprises SEQ ID NO: 130, and wherein the antisense strand comprises SEQ ID NO: 131.

E88. The RNAi oligonucleotide of any one of embodiments 69 to 77, wherein the sense strand comprises SEQ ID NO: 132, and wherein the antisense strand comprises SEQ ID NO: 133.

E89. The RNAi oligonucleotide of any one of embodiments 69 to 77, wherein the sense strand comprises SEQ ID NO: 134, and wherein the antisense strand comprises SEQ ID NO: 135.

E90. The RNAi oligonucleotide of any one of embodiments 69 to 77, wherein the sense strand comprises SEQ ID NO: 136, and wherein the antisense strand comprises SEQ ID NO: 137.

E91. The RNAi oligonucleotide of any one of embodiments 69 to 77, wherein the sense strand comprises SEQ ID NO: 138, and wherein the antisense strand comprises SEQ ID NO: 139.

E92. The RNAi oligonucleotide of any one of embodiments 69 to 77, wherein the sense strand comprises SEQ ID NO: 140, and wherein the antisense strand comprises SEQ ID NO: 141.

E93. The RNAi oligonucleotide of any one of embodiments 69 to 77, wherein the sense strand comprises SEQ ID NO: 142, and wherein the antisense strand comprises SEQ ID NO: 143.

E94. The RNAi oligonucleotide of any one of embodiments 69 to 77, wherein the sense strand comprises SEQ ID NO: 144, and wherein the antisense strand comprises SEQ ID NO: 145.

E95. The RNAi oligonucleotide of any one of embodiments 69 to 77, wherein the sense strand comprises SEQ ID NO: 146, and wherein the antisense strand comprises SEQ ID NO: 147.

E96. The RNAi oligonucleotide of any one of embodiments 69 to 77, wherein the sense strand comprises SEQ ID NO: 191, and wherein the antisense strand comprises SEQ ID NO: 192.

E97. A method for treating a subject having a disease, disorder or condition associated with PLP1 expression, the method comprising administering to the subject a therapeutically effective amount of the RNAi oligonucleotide of any one of the preceding embodiments, or pharmaceutical composition thereof, thereby treating the subject.

E98. The method of embodiment 97, wherein the RNAi oligonucleotide is administered intrathecally, intracerebroventricularly, or by intracisternal magna injection.

E99. The method of embodiment 97 or 98, wherein a single dose of the RNAi oligonucleotide is administered.

E100. The method of embodiment 97 or 98, wherein more than one dose of the RNAi oligonucleotide is administered.

E101. The method of any one of embodiments 97-100, wherein PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks.

E102. The method of any one of embodiments 97-100, wherein PLP1 expression is reduced for about 1 month, 2 months, 3 months, 4 months, 5 months or 6 months.

E103. The method of any one of embodiments 97-100, wherein PLP1 expression is reduced for about 7 days, 14 days, 21 days, 28 days, 35 days, 42 days, 49 days, 56 days, 63 days, days, 77 days, 84 days, or 91 days.

E104. A pharmaceutical composition comprising the RNAi oligonucleotide of any one of embodiments 1 to 96, and a pharmaceutically acceptable carrier, delivery agent or excipient.

E105. A method of delivering an oligonucleotide to a subject, the method comprising administering pharmaceutical composition of embodiment 104 to the subject.

E106. A method for reducing PLP1 expression in a cell, a population of cells or a subject, the method comprising the step of:
i. contacting the cell or the population of cells with the RNAi oligonucleotide of any one of embodiments 1 to 96, or the pharmaceutical composition of embodiment 104; or
ii. administering to the subject the RNAi oligonucleotide of any one of embodiments 1 to 83, or the pharmaceutical composition of embodiment 85.

E107. The method of embodiment 106, wherein reducing PLP1 expression comprises reducing an amount or level of PLP1 mRNA, an amount or level of PLP1 protein, or both.

E108. The method of embodiment 106, wherein PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks.

E109. The method of embodiment 106, wherein PLP1 expression is reduced for about 1 month, 2 months, 3 months, 4 months, 5 months or 6 months.

E110. The method of embodiment 106, wherein PLP1 expression is reduced for about 7 days, 14 days, 21 days, 28 days, 35 days, 42 days, 49 days, 56 days, 63 days, 70 days, 77 days, 84 days, or 91 days.

E111. The method of any one of embodiments 106-110, wherein the subject has a disease, disorder or condition associated with PLP1 expression.

E112. The method of embodiment 97 or 111, wherein the disease, disorder or condition associated with PLP1 expression is Pelizaeus-Merzbacher disease (PMD) or spastic paraplegia type 2 (SPG2).

E113. The method of any one of embodiments 97 and 105 to 112, wherein the RNAi oligonucleotide, or pharmaceutical composition, is administered in combination with a second composition or therapeutic agent.

E114. A method for treating a subject having a disease, disorder or condition associated with PLP1 expression, the method comprising administering to the subject a therapeutically effective amount of an RNAi oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 171-188, and wherein the region of complementarity is at least 15 contiguous nucleotides in length.

E115. The method of embodiment 114, wherein the region of complementarity differs by no more than 3 nucleotides in length to the PLP1 mRNA target sequence.

E116. The method of embodiment 114, wherein the region of complementarity is fully complementary to the PLP1 mRNA target sequence.

E117. A method for treating a subject having a disease, disorder or condition associated with PLP1 expression, the method comprising administering to the subject a therapeutically effective amount of an RNAi oligonucleotide comprising a sense strand and an antisense strand selected from a row set forth in Table 5, or pharmaceutical composition thereof, thereby treating the subject.

E118. A method for treating a subject having a disease, disorder or condition associated with PLP1 expression, the method comprising administering to the subject a therapeutically effective amount of an RNAi oligonucleotide comprising a sense strand and an antisense strand, wherein the sense strand and antisense strands comprise nucleotide sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

E119. A method for treating a subject having a disease, disorder or condition associated with PLP1 expression, the method comprising administering to the subject a therapeutically effective amount of an RNAi oligonucleotide comprising a sense strand and an antisense strand, wherein the sense strand and antisense strands are selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively;
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively; and
(s) SEQ ID NOs: 191 and 192, respectively.

E120. The method of embodiment 119, wherein the sense strand comprises SEQ ID NO: 112, and wherein the anti sense strand comprises SEQ ID NO: 113.

E121. The method of embodiment 119, wherein the sense strand comprises SEQ ID NO: 114, and wherein the anti sense strand comprises SEQ ID NO: 115.

E122. The method of embodiment 119, wherein the sense strand comprises SEQ ID NO: 116, and wherein the anti sense strand comprises SEQ ID NO: 117.

E123. The method of embodiment 119, wherein the sense strand comprises SEQ ID NO: 118, and wherein the anti sense strand comprises SEQ ID NO: 119.

E124. The method of embodiment 119, wherein the sense strand comprises SEQ ID NO: 120, and wherein the anti sense strand comprises SEQ ID NO: 121.

E125. The method of embodiment 119, wherein the sense strand comprises SEQ ID NO: 122, and wherein the antisense strand comprises SEQ ID NO: 123.

E126. The method of embodiment 119, wherein the sense strand comprises SEQ ID NO: 124, and wherein the antisense strand comprises SEQ ID NO: 125.

E127. The method of embodiment 119, wherein the sense strand comprises SEQ ID NO: 126, and wherein the antisense strand comprises SEQ ID NO: 127.

E128. The method of embodiment 119, wherein the sense strand comprises SEQ ID NO: 128, and wherein the antisense strand comprises SEQ ID NO: 129.

E129. The method of embodiment 119, wherein the sense strand comprises SEQ ID NO: 130, and wherein the anti sense strand comprises SEQ ID NO: 131.

E130. The method of embodiment 119, wherein the sense strand comprises SEQ ID NO: 132, and wherein the anti sense strand comprises SEQ ID NO: 133.

E131. The method of embodiment 119, wherein the sense strand comprises SEQ ID NO: 134, and wherein the anti sense strand comprises SEQ ID NO: 135.

E132. The method of embodiment 119, wherein the sense strand comprises SEQ ID NO: 136, and wherein the anti sense strand comprises SEQ ID NO: 137.

E133. The method of embodiment 119, wherein the sense strand comprises SEQ ID NO: 138, and wherein the anti sense strand comprises SEQ ID NO: 139.

E134. The method of embodiment 119, wherein the sense strand comprises SEQ ID NO: 140, and wherein the anti sense strand comprises SEQ ID NO: 141.

E135. The method of embodiment 119, wherein the sense strand comprises SEQ ID NO: 142, and wherein the antisense strand comprises SEQ ID NO: 143.

E136. The method of embodiment 119, wherein the sense strand comprises SEQ ID NO: 144, and wherein the antisense strand comprises SEQ ID NO: 145.

E137. The method of embodiment 119, wherein the sense strand comprises SEQ ID NO: 146, and wherein the antisense strand comprises SEQ ID NO: 147.

E138. The method of embodiment 119, wherein the sense strand comprises SEQ ID NO: 191, and wherein the anti sense strand comprises SEQ ID NO: 192.

E139. The method of any one of embodiments 114-138, wherein the disease, disorder or condition associated with PLP1 expression is Pelizaeus-Merzbacher disease (PMD) or spastic paraplegia type 2 (SPG2).

E140. The method of any one of embodiments 114-139, wherein the RNAi oligonucleotide is administered intrathecally, intracerebroventricularly, or by intracisternal magna injection.

E141. The method of any one of embodiments 114-140, wherein a single dose of the RNAi oligonucleotide is administered.

E142. The method of any one of embodiments 114-140, wherein more than one dose of the RNAi oligonucleotide is administered.

E143. The method of any one of embodiments 114-142, wherein PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks.

E144. The method of any one of embodiments 114-142, wherein PLP1 expression is reduced for about 1 month, 2 months, 3 months, 4 months, 5 months or 6 months.

E145. The method of any one of embodiments 114-142, wherein PLP1 expression is reduced for about 7 days, 14 days, 21 days, 28 days, 35 days, 42 days, 49 days, 56 days, 63 days, 70 days, 77 days, 84 days, or 91 days.

E146. Use of the RNAi oligonucleotide of any one of embodiments 1 to 96, or the pharmaceutical composition of embodiment 104, in the manufacture of a medicament for the treatment of a disease, disorder or condition associated with PLP1 expression, optionally for the treatment of Pelizaeus-Merzbacher disease (PMD) or spastic paraplegia type 2 (SPG2).

E147. The RNAi oligonucleotide of any one of embodiments 1 to 96, or the pharmaceutical composition of embodiment 104, for use, or adaptable for use, in the treatment of a disease, disorder or condition associated with PLP1 expression, optionally for the treatment of Pelizaeus-Merzbacher disease (PMD) or spastic paraplegia type 2 (SPG2).

E148. A kit comprising the RNAi oligonucleotide of any one of embodiments 1 to 96, an optional pharmaceutically acceptable carrier, and a package insert comprising instructions for administration to a subject having a disease, disorder or condition associated with PLP1 expression.

E149. The use of embodiment 146, the RNAi oligonucleotide or pharmaceutical composition for use, or adaptable for use, of embodiment 147, or the kit of embodiment 148, wherein the disease, disorder or condition associated with PLP1 expression is Pelizaeus-Merzbacher disease (PMD) or spastic paraplegia type 2 (SPG2).

E150. A composition comprising an RNAi oligonucleotide for reducing PLP1 expression and a pharmaceutically acceptable carrier, wherein the oligonucleotide comprises a sense strand and an antisense strand that form a duplex region, wherein the antisense strand comprises a region of complementarity to a PLP1 mRNA target sequence, wherein the region of complementarity is at least 15 contiguous nucleotides in length, and wherein the composition is formulated for administration to the cerebral spinal fluid (CSF) of a subject.

E151. The composition of embodiment 150 comprising the RNAi oligonucleotide of any one of embodiments 1-96.

E152. The composition of embodiment 150 or 151, wherein the composition is formulated for intrathecal, intracerebroventricular, or intracisternal magna administration.

E153. The composition of any one of embodiments 150-1152, wherein the oligonucleotide does not comprise a targeting ligand.

E154. The composition of any one of embodiments 150-153, wherein the oligonucleotide is not formulated in a lipid, liposome or lipid nanoparticle delivery vehicle.

E155. The composition of any one of embodiments 150-154, wherein the pharmaceutically acceptable carrier comprises phosphate buffered saline.

E156. A method for reducing expression of HY 1 in the central nervous system of a subject, comprising administering a composition comprising an RNAi oligonucleotide and a pharmaceutically acceptable carrier, wherein the RNAi oligonucleotide comprises a sense strand and an antisense strand that form a duplex region, wherein the antisense strand comprises a region of complementarity to a PLP1 mRNA, wherein the region of complementarity is at least 15 contiguous nucleotides in length, and wherein the composition is formulated for administration to the cerebral spinal fluid (CSF), thereby reducing PLP1 expression in the central nervous system.

E157. The method of embodiment 156, wherein the composition comprises a RNAi oligonucleotide of any one of embodiments 1-96.

E158. The method of any one of embodiments 156-157, wherein a single dose or more than one dose is administered.

E159. The method of any one of embodiments 156-158, wherein PLP1 expression is reduced for about 1 week, 2 weeks, 3 weeks, 4 weeks, 5 weeks, 6 weeks, 7 weeks, 8 weeks, 9 weeks, 10 weeks, 11 weeks, or 12 weeks.

E160. The method of any one of embodiments 156-158, wherein PLP1 expression is reduced for about 1 month, 2 months, 3 months, 4 months, 5 months or 6 months.

E161. The method of any one of embodiments 156-158, wherein PLP1 expression is reduced for about 7 days, 14 days, 21 days, 28 days, 35 days, 42 days, 49 days, 56 days, 63 days, 70 days, 77 days, 84 days, or 91 days.

E162. The method of any one of embodiments 156-161, wherein PLP1 expression is reduced in at least one region of the brain.

E163. The method of embodiment 162, wherein the at least one region of the brain is selected from: frontal cortex, parietal cortex, temporal cortex, occipital cortex and cerebellum.

E164. The method of any one of embodiments 156-163, wherein PLP1 expression is reduced in the cervical spinal cord, thoracic spinal cord, lumbar spinal cord, and/or lumbar dorsal root ganglion.

E165. The method of any one of embodiments 156-164, wherein the composition and/or the oligonucleotide does not comprise a targeting ligand.

E166. The method of any one of embodiments 156-165, wherein the oligonucleotide is not formulated in a lipid, liposome or lipid nanoparticle delivery vehicle.

E167. The method of any one of embodiments 156-166, wherein the pharmaceutically acceptable carrier comprises phosphate buffered saline.

### Definitions

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a condition in the subject).

As used herein, “asialoglycoprotein receptor” or “ASGPR” refers to a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of a disease associated with PLP expression (e.g., PMD) in a subject. This attenuation may be exemplified by, for example, a decrease in one or more aspects (e.g., symptoms, tissue characteristics, and cellular, inflammatory or immunological activity, etc.) of a disease associated with PLP expression (e.g., PMD), no detectable progression (worsening) of one or more aspects of the disease, or no detectable aspects of the disease in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “labile linker” refers to a linker that can be cleaved (e.g., by acidic pH). A “fairly stable linker” refers to a linker that cannot be cleaved.

As used herein, “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a dsRNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or single-stranded siRNA. In some embodiments, a double-stranded oligonucleotide is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotides.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “PLP” and “PLP1” are used interchangeably and refer to myelin proteolipid protein or proteolipid protein 1. The PLP1 gene encodes two protein isoforms (PLP and DM20) which represent the predominant protein portion in myelin of the central nervous system. The two products are generated from the same primary transcript by alternative splicing. PLP1 is found primarily in nerves in the central nervous system and DM20 is produced mainly in nerves that connect the brain and spinal cord to muscles (peripheral nervous system). These two proteins are found within the cell membrane of nerve cells, where they make up the majority of myelin and anchor it to the cells. The amino acid sequence for human PLP is set forth in SEQ ID NO: 189. The mRNA encoding wild-type human PLP is set forth in SEQ ID NO: 1.

As used herein, “reduced expression” of a gene (e.g., PLP1) refers to a decrease in the amount or level of RNA transcript (e.g., PLP1 mRNA) or protein encoded by the gene and/or a decrease in the amount or level of activity of the gene in a cell, a population of cells, a sample or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising PLP1 mRNA) may result in a decrease in the amount or level of PLP1 mRNA, PLP2 protein and/or PLP1 activity (e.g., via inactivation and/or degradation of PLP/mRNA by the RNAi pathway) when compared to a cell that is not treated with the double-stranded oligonucleotide. Similarly, and as used herein, “reducing expression” refers to an act that results in reduced expression of a gene (e.g., PLP1).

As used herein, “reduction of PLP1 expression” refers to a decrease in the amount or level of PLP1 mRNA, PLP1 protein and/or PLP1 activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., PLP1 mRNA) or (b) a single-stranded oligonucleotide having a single antisense strand, where that antisense strand (or part of that antisense strand) is used by the Ago2 endonuclease in the cleavage of a target mRNA (e.g., PLP1 mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) Nature 346:680-682; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In certain embodiments, a tetraloop comprises or consists of 3, 4, 5 or 6 nucleotides, which may or may not be modified (e.g., which may or may not be conjugated to a targeting ligand). In one embodiment, a tetraloop consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), T (thymine) or U (uracil) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:4281-92; Shinji et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

### Example 1: Generation of PLP1-Targeting Double-Stranded (DS) RNAi Oligonucleotides

Proteolipid protein 1 (PLP1) is a transmembrane myelin proteolipid that is the predominant myelin protein found in the CNS. Mutations in the PLP1 gene can lead to various diseases, including Pelizaeus-Merzbacher Disease (PMD). Oligonucleotides capable of inhibiting PLP1 mRNA expression were identified and generated.

Identification of PLP1 mRNA Target Sequences

Double-stranded RNAi oligonucleotides described herein that target murine Plp1, non-human primate (NHP or “monkey”) PLP1, and/or human PLP1 mRNA and inhibit mRNA expression via the RNAi pathway are referred to henceforth in the Examples as “PLP1 RNAi oligonucleotides”. The term “PLP1 expression” as used in the Examples refers to murine Plp1, non-human primate (NHP or “monkey”) PLP1, and/or human PLP1 mRNA expression. The term “PLP1 mRNA target sequence” as used in the Examples refers to a murine Plp1, a non-human primate (NHP or “monkey”) PLP1, and/or a human PLP1 mRNA target sequence. To generate PLP1 RNAi oligonucleotides, a computer-based algorithm was used to computationally identify PLP1 mRNA target sequences suitable for assaying inhibition of PLP1 expression by the RNAi pathway. The algorithm provided RNAi oligonucleotide guide (antisense) strand sequences each having a region of complementarity to a suitable PLP1 mRNA target sequence of human (Hs) or murine (Mm) mRNA (e.g., SEQ ID NOs: 1 and 2, respectively; Table 1). Due to sequence conservation across species, some of the PLP1 mRNA target sequences identified for human PLP1 mRNA are homologous to the corresponding PLP1 mRNA target sequence of murine (mM) Plp1 mRNA (SEQ ID NO: 2; Table 1) and/or monkey (Mf) PLP1 mRNA (SEQ ID NO: 3; Table 1). PLP1 RNAi oligonucleotides comprising a region of complementarity to homologous PLP1 mRNA target sequences with nucleotide sequence similarity are predicted to have the ability to target homologous PLP1 mRNAs (e.g., human PLP1 and monkey PLP1 mRNAs). Exemplary PLP1 mRNA target sequences are provided in Table 2.

TABLE 1
Exemplary Human PLP1, Monkey PLP1, and Mouse Plp1 mRNA Sequences

| Species | GenBank Ref Seq # | SEQ ID NO |
|---|---|---|
| Human (Hs) | NM_001128834.2 | 1 |
| Mouse (Mm) | NM_011123.4 | 2 |
| Cynomolgus monkey (Mf) | NM_001283166.1 | 3 |

TABLE 2
Exemplary PLP1 mRNA Target Sequences

| | | SEQ ID |
|---|---|---|
| Target Sequence | Species | NO |
| AUGAGUAUCUCAUUAAUGUAAUUCA | Mm | 148 |
| AGUAUCUCAUUAAUGUGAUACAUGC | Mm | 149 |
| AUUAAUGUGAUUCAUGCUUACCAGT | Mm | 150 |
| GAGCAUAGUUCUUUUUGAAAACAAG | Mm | 151 |
| AGCAUAGUUCUUUUUGAAAACAAGA | Mm | 152 |
| AGAAAGCAUCACAAAAAUAAUUGAA | Mm | 153 |
| GAAAGCAUCACAAAAAUAUAUGAAA | Mm | 154 |
| CAUCACAAAAAUAUUUGAAAUUGTA | Mm | 155 |
| ACAGAUGAUUUUACUUGCUAAUATT | Mm | 156 |
| CAGAUGAUUUUACUUGCUAAUAUTA | Mm | 157 |
| AUUUUACUUGCUAAUAUUAACUCAG | Mm | 158 |
| AAGUUACUGUCUCUUGGUAAAUATA | Mm | 159 |
| GGAAAAGUUAUUGUAGCUGAUUCAT | Mm | 160 |
| GAAAAGUUAUUGUAGCUGUAUCATT | Mm | 161 |
| AAAGUUAUUGUAGCUGUUUAAUUGT | Mm | 162 |
| AAGUUAUUGUAGCUGUUUCAUUGTA | Mm | 163 |
| GAAGGUGAAAUAAUCUAUAACUUTT | Mm | 164 |
| GUUUUGGUUUAAUAUAACAAAUAAC | Mm | 165 |
| GAUAGAGAAUUUUGAUUUUAACAAC | Mm | 166 |
| AUAGAGAAUUUUGAUUUUAACAACA | Mm | 167 |
| AGAAUUUUGAUUUUAACAAAAUAAA | Mm | 168 |
| AGUGAAUUGUUCUAUUUGAACUCAA | Mm | 169 |
| GUGAAUUGUUCUAUUUGACAUCAAT | Mm | 170 |
| ACAGAAAAGCUAAUUGAGACCUAUU | Hs-Mf-Mm | 171 |
| CAGAAAAGCUAAUUGAGACCUAUUU | Hs-Mf-Mm | 172 |
| GCUAAUUGAGACCUAUUUCUCCAAA | Hs-Mf-Mm | 173 |
| CUAAUUGAGACCUAUUUCUCCAAAA | Hs-Mf-Mm | 174 |
| GACUAUGAGUAUCUCAUCAAUGUGA | Hs-Mf | 175 |
| ACUAUGAGUAUCUCAUCAAUGUGAU | Hs-Mf | 176 |
| AUGAGUAUCUCAUCAAUGUGAUCCA | Hs-Mf | 177 |
| GAGUAUCUCAUCAAUGUGAUCCAUG | Hs-Mf | 178 |
| AGUAUCUCAUCAAUGUGAUCCAUGC | Hs-Mf | 179 |
| CUGUGCCUGUGUACAUUUACUUCAA | Hs-Mf | 180 |
| CUGUGUACAUUUACUUCAACACCUG | Hs-Mf-Mm | 181 |
| GUGUACAUUUACUUCAACACCUGGA | Hs-Mf | 182 |
| CCAGAAUGUAUGGUGUUCUCCCAUG | Hs-Mf-Mm | 183 |
| CAGCUGAGUUCCAAAUGACCUUCCA | Hs-Mf-Mm | 184 |
| AAUGACCUUCCACCUGUUUAUUGCU | Hs-Mf-Mm | 185 |
| GACCUUCCACCUGUUUAUUGCUGCA | Hs-Mf-Mm | 186 |
| GCUCACCUUCAUGAUUGCUGCCACU | Hs-Mf-Mm | 187 |
| ACCUUCAUGAUUGCUGCCACUUACA | Hs-Mf-Mm | 188 |
| ACAGAAAAGCUAAUUGAGA | Hs-Mf-Mm | 212 |
| CAGAAAAGCUAAUUGAGAC | Hs-Mf-Mm | 213 |
| GAAAAGCUAAUUGAGACCU | Hs-Mf-Mm | 214 |
| GCUAAUUGAGACCUAUUUC | Hs-Mf-Mm | 215 |
| CUAAUUGAGACCUAUUUCU | Hs-Mf-Mm | 216 |
| GACUAUGAGUAUCUCAUCA | Hs-Mf | 217 |
| ACUAUGAGUAUCUCAUCAA | Hs-Mf | 218 |
| AUGAGUAUCUCAUCAAUGU | Hs-Mf | 219 |
| GAGUAUCUCAUCAAUGUGA | Hs-Mf | 220 |
| AGUAUCUCAUCAAUGUGAU | Hs-Mf | 221 |
| CGGGUGUGUCAUUGUUUGG | Hs-Mf | 222 |
| CUGUGCCUGUGUACAUUUA | Hs-Mf | 223 |
| CUGUGUACAUUUACUUCAA | Hs-Mf-Mm | 224 |
| GUGUACAUUUACUUCAACA | Hs-Mf | 225 |
| CCAGAAUGUAUGGUGUUCU | Hs-Mf-Mm | 226 |
| CAGCUGAGUUCCAAAUGAC | Hs-Mf-Mm | 227 |
| AAUGACCUUCCACCUGUUU | Hs-Mf-Mm | 228 |
| GACCUUCCACCUGUUUAUU | Hs-Mf-Mm | 229 |
| GCUCACCUUCAUGAUUGCU | Hs-Mf-Mm | 230 |
| ACCUUCAUGAUUGCUGCCA | Hs-Mf-Mm | 231 |
| ACAGAUGAUUUUACUUGCU | Mm | 232 |
| CAGAUGAUUUUACUUGCUA | Mm | 233 |
| AAGUUACUGUCUCUUGGUA | Mm | 234 |

Hs-Mf = human/monkey homologous PLP1 mRNA target sequence
Hs-Mf-Mm = human/monkey/mouse homologous PLP1 mRNA target sequence

PLP1 RNAi oligonucleotides were synthesized as described below, each having a unique antisense (guide) strand comprising a region of complementarity to a PLP1 mRNA target sequence identified by the algorithm and having a corresponding passenger (sense) strand comprising the PLP1 mRNA target sequence identified by the algorithm.

### Synthesis of PLP1 RNAi Oligonucleotides

The PLP1 RNAi oligonucleotides were chemically synthesized using methods described herein. Specifically, RNA oligonucleotides were synthesized using solid phase oligonucleotide synthesis methods generally as described for 19-23mer siRNAs (see, e.g., Scaringe et al. (1990) NUCLEIC ACIDS RES. 18:5433-5441 and Usman et al. (1987) J. Am. CHEM. SOC. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158).

Sense and antisense strands were separately synthesized as individual RNA oligonucleotides and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-2684). The RNA oligonucleotides were purified using ion-exchange high performance liquid chromatography (IE-HPLC) on an Amersham Source 15Q column (1.0 cm×25 cm; Amersham Pharmacia Biotech) using a 15-minute step-linear gradient. The gradient varies from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length RNA oligonucleotide species were collected, pooled, desalted on NAP-5 columns, and lyophilized.

The purity of each RNA oligonucleotide was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of RNA oligonucleotide was injected into a capillary, run in an electric field of 444 V/cm and detected by UV absorbance at 260 nm. Denaturing Tris-Borate-7 M-urea running buffer was purchased from Beckman-Coulter. RNA oligonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all RNA oligonucleotides were obtained, often within 0.2% of expected molecular mass.

Single-stranded RNA oligonucleotides were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The double-stranded RNAi oligonucleotides were stored at −20° C. Single-stranded RNA oligonucleotides were stored lyophilized or in nuclease-free water at −80° C.

### Example 2: PLP1 RNAi Oligonucleotides Inhibit Murine Plp1 mRNA Expression in the Central Nervous System

To evaluate the ability of PLP1 RNAi oligonucleotides generated by the methods described in Example 1 to inhibit PLP1 expression in the central nervous system (CNS), mice were treated with PLP1 RNAi oligonucleotides that target murine Plp1 mRNA. Briefly, the PLP1 mRNA target sequences provided in Table 2 were used to generate eighteen (18) PLP1 RNAi oligonucleotides that target murine Plp1 mRNA (Table 3), each comprising a nicked tetraloop structure having a 36-mer passenger strand and a 22-mer guide strand.

TABLE 3
PLP1 RNAi Oligonucleotides Targeting Murine Plp1 mRNA

| | | SEQ ID NO | SEQ ID NO | SEQ ID NO | SEQ ID NO |
|---|---|---|---|---|---|
| Oligo- | | (Sense) | (Antisense) | (Sense) | (Antisense) |
| nucleotide | DP# | Unmodified | | Modified | |
| PLP1-353 | DP17453P:DP17452G | 4 | 5 | 40 | 41 |
| PLP1-356 | DP17443P:DP17442G | 6 | 7 | 42 | 43 |
| PLP1-364 | DP17447P:DP17446G | 8 | 9 | 44 | 45 |
| PLP1-2191 | DP17437P:DP17436G | 10 | 11 | 46 | 47 |
| PLP1-2192 | DP17439P:DP17438G | 12 | 13 | 48 | 49 |
| PLP1-2197 | DP17425P:DP17424G | 14 | 15 | 50 | 51 |
| PLP1-2339 | DP17427P:DP17426G | 16 | 17 | 52 | 53 |
| PLP1-2340 | DP17449P:DP17448G | 18 | 19 | 54 | 55 |
| PLP1-2346 | DP17441P:DP17440G | 20 | 21 | 56 | 57 |
| PLP1-2398 | DP17429P:DP17428G | 22 | 23 | 58 | 59 |
| PLP1-2779 | DP17457P:DP17456G | 24 | 25 | 60 | 61 |
| PLP1-2780 | DP17435P:DP17434G | 26 | 27 | 62 | 63 |
| PLP1-2977 | DP17445P:DP17444G | 28 | 29 | 64 | 65 |
| PLP1-3007 | DP17455P:DP17454G | 30 | 31 | 66 | 67 |
| PLP1-3130 | DP17431P:DP17430G | 32 | 33 | 68 | 69 |
| PLP1-3134 | DP17451P:DP17450G | 34 | 35 | 70 | 71 |
| PLP1-3254 | DP17423P:DP17422G | 36 | 37 | 72 | 73 |
| PLP1-3255 | DP17433P:DP17432G | 38 | 39 | 74 | 75 |

The passenger strand and guide strand of the PLP1 RNAi oligonucleotides provided in Table 3 each comprise a distinct pattern of modified nucleotides and phosphorothioate linkages (SEQ ID Nos: 40-75). The pattern of modified nucleotides and phosphorothioate linkages is illustrated below:

Sense Strand:
5′-mX-S-mX-fX-mX-fX-mX-mX-fX-mX-fX-mX-fX-fX-mX-
fX-mX-fX-mX-mX-mX-mX-S-mX-S-mX-S-mX-S-mX-S-mX-S-
mX-S-mX-S-mX-S-mX-S-mX-S-mX-S-mX-S-mX-S-mX-S-mX-3′

### Hybridized to:

Antisense Strand:
5′-[MePhosphonate-40-mX]-S-fX-S-fX-fX-fX-mX-fX-mX-mX-fX-mX-
mX-mX-fX-mX-fX-mX-mX-fX-mX-S-mX-S-mX-3′
(Modification key: Table 4).

TABLE 4
Modification Key

| Symbol | Modification/linkage |
|---|---|
| mX | 2′-O-methyl modified nucleotide |
| fX | 2′-fluoro modified nucleotide |
| -S- | phosphorothioate linkage |
| — | phosphodiester linkage |
| [MePhosphonate-4O-mX] | 5′-methoxyphosphonate-4-oxy modified nucleotide |

The PLP1 RNAi oligonucleotides provided in Table 3 were administered via intrathecal injection (10 IA) into the lumbar spine of C57/BL6 female mice age 6-8 weeks old at a dose of 250 μg (10 mg/kg) formulated in phosphate buffered saline (PBS) (n=5). A control group of mice (n=5) were administered only PBS. Seven (7) days post-injection, mice were sacrificed using CO₂ asphyxiation followed by decapitation. Whole brain and lumbar spinal cord were dissected and preserved for RT-qPCR analysis. RNA was extracted to determine PLP1 mRNA levels by qPCR (normalized to endogenous housekeeping genes Rp123 or Gapdh, as indicated). The levels of PLP1 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to the region of PLP1 mRNA spanning exons 4-6. The percentage of PLP1 mRNA remaining in samples from the lumbar spinal cord and frontal cortex of mice treated with PLP1 RNAi oligonucleotides was determined using the 2^{-ΔΔCt} (“delta-delta Ct”) method (Livak and Schmittgen (2001) METHODS 25:402-408).

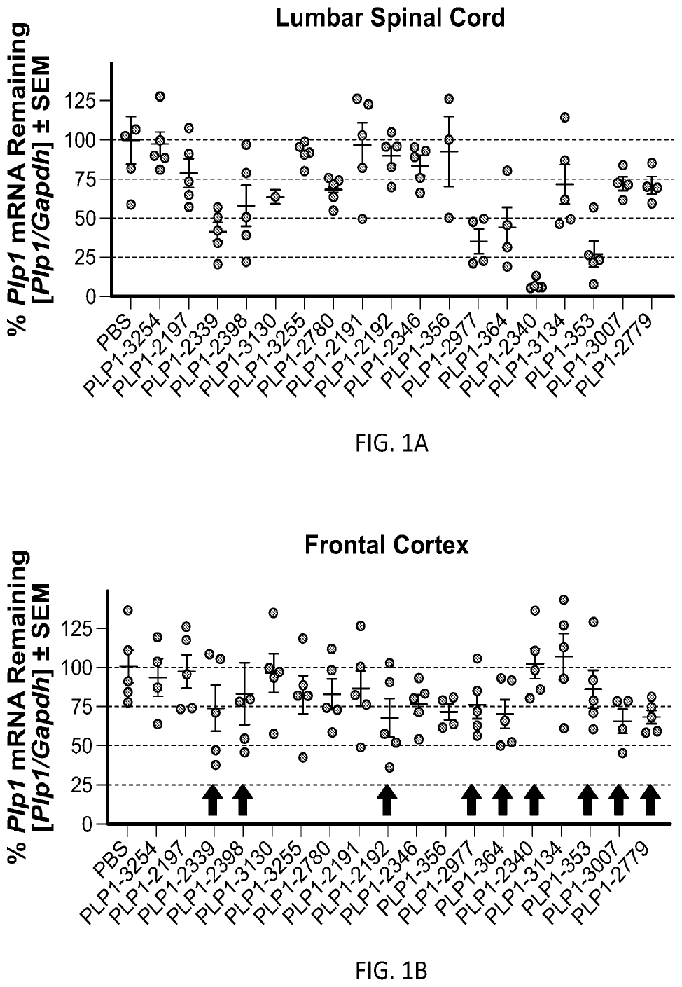

As shown in FIGS. 1A and 1B, multiple PLP1 RNAi oligonucleotides inhibited PLP1 expression in the CNS of mice at regions proximal (e.g. lumbar spinal cord; FIG. 1A) and distal to the site of injection (e.g., frontal cortex; FIG. 1B). Inhibition of PLP1 expression was determined by comparing the percentage of PLP1 mRNA remaining in samples from mice treated with PLP1 RNAi oligonucleotides relative to the percentage of PLP1 mRNA remaining in samples from control mice treated with PBS. These results demonstrate that PLP1 RNAi oligonucleotides inhibit PLP1 expression in different anatomical regions of the CNS following intrathecal injection into the lumbar spine. The PLP1 RNAi oligonucleotides PLP-2339, PLP1-2398, and PLP1-2340 were selected for further evaluation (Example 3).

### Example 3: PLP1 RNAi Oligonucleotides Inhibit Murine Plp1 Expression in a Dose-Dependent Manner

### Phosphorothioate Modified Tetraloop

To further evaluate the ability of PLP1 RNAi oligonucleotides described in Example 2 to inhibit PLP1 expression in the CNS, mice were injected with PLP1 RNAi oligonucleotides at two different dose levels. Specifically, C57/BL6 female mice age 6 to 8 weeks old were injected with a subset of the PLP1 RNAi oligonucleotides described in Example 2 (i.e. phosphorothioate modified tetraloop). In separate treatment groups, three (3) PLP1 RNAi oligonucleotides (PLP1-2339; sense strand SEQ ID NO: 52, antisense strand SEQ ID NO: 53, PLP1-2398; sense strand SEQ ID NO: 58, antisense strand SEQ ID NO: 59, and PLP1-2340; sense strand SEQ ID NO: 54, antisense strand SEQ ID NO: 55) formulated in PBS were administered by intrathecal injection into the lumbar spine at 100 μg or 250 μg doses (4 mg/kg or 10 mg/kg dose level) (n=5 per treatment group). A control group of mice (n=5) were administered only PBS. After seven (7) days post-injection, mice were sacrificed using CO₂ asphyxiation followed by decapitation. Whole brain and lumbar spinal cord were dissected and preserved for RT-qPCR analysis. RNA was extracted and measured as described in Example 2 to determine PLP1 mRNA levels in the lumbar spinal cord, brain stem, hippocampus and frontal cortex.

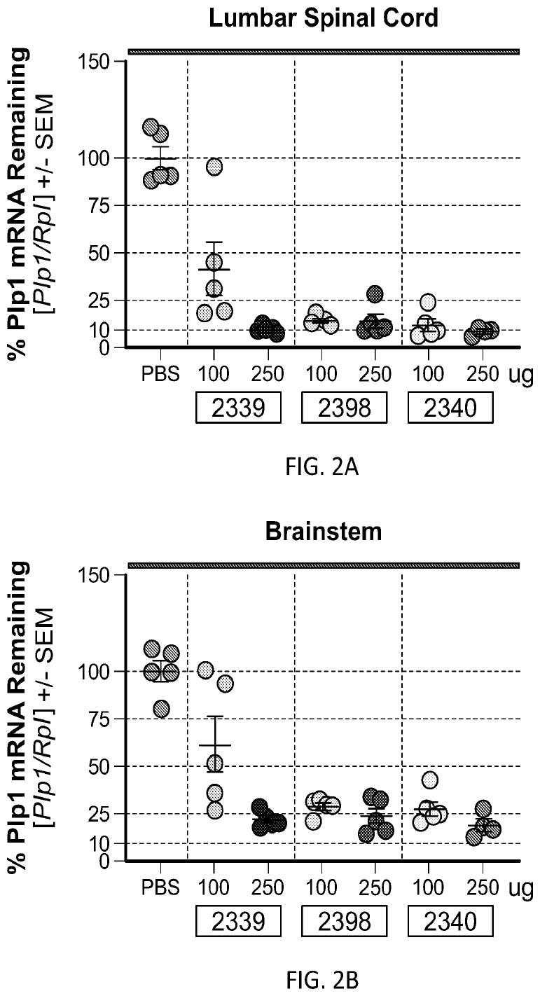

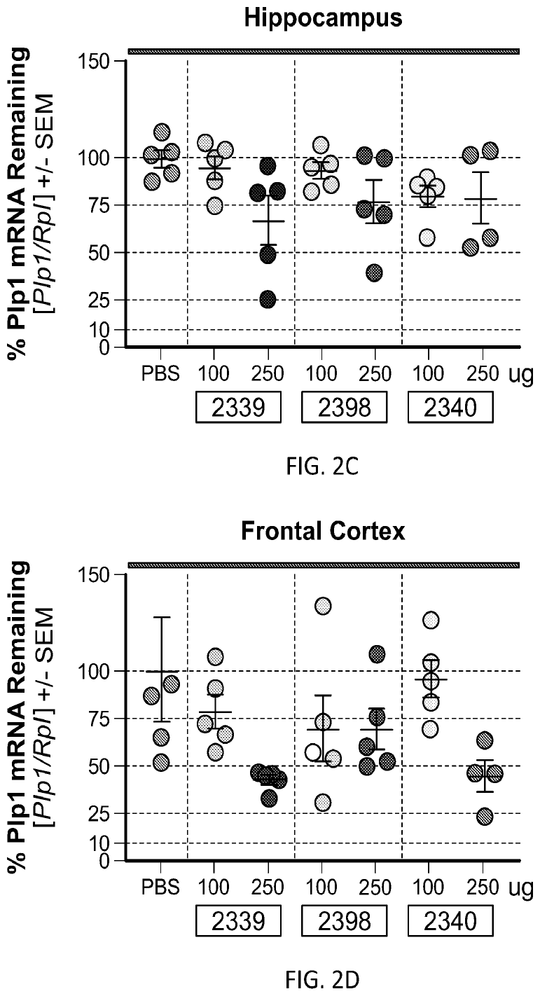

As shown in FIGS. 2A-2D, the indicated PLP1 RNAi oligonucleotides inhibited PLP1 expression in the CNS of mice at regions proximal (e.g., lumbar spinal cord, brain stem; FIGS. 2A-2B) and distal to the site of injection (e.g., hippocampus; frontal cortex; FIGS. 2C-2D), consistent with the results described in Example 2. Further, PLP1-2339 inhibited expression in a dose-dependent manner in regions both proximal and distal to the injection site (e.g., lumbar spinal cord, brain stem, hippocampus and frontal cortex), while the inhibition of PLP1 expression by PLP1-2398 and PLP1-2340 remained approximately equal at both doses in regions proximal to the injection site (e.g., lumbar spinal cord; brain stem). As in Example 2, inhibition of PLP1 expression was determined by comparing the percentage of PLP1 mRNA remaining in samples from mice treated with PLP1 RNAi oligonucleotides relative to the percentage of PLP1 mRNA remaining in samples from control mice treated with PBS. These results demonstrate that PLP1 RNAi oligonucleotides inhibit PLP1 expression in a dose-dependent manner in the CNS following intrathecal injection into the lumbar spine.

### GalNAc Modified Tetraloop

Following validation of delivery to the CNS with a phosphorothioate modified tetraloop, it was evaluated whether additional modification patterns would deliver the described RNAi oligonucleotides to the CNS. Therefore, the 3 oligonucleotides tested above (PLP1-2339, PLP1-2398, and PLP1-2340) were modified using the pattern depicted in FIG. 3. Specifically, three of the nucleotides comprising the tetraloop were each conjugated to a GalNAc moiety (CAS #14131-60-3). The molecular structure of which is illustrated below:

Sense Strand:
5′ mX-S-mX-mX-mX-mX-mX-mX-fX-fX-fX-fX[-mX-]₁₆-[ademX-GalNAc]-
[ademX-GalNAc]-[ademX-GalNAc]-mX-mX-mX-mX-mX-mX 3′
Hybridized to:
Antisense Strand:
5′ [MePhosphonate-40-mX]-S-fX-S-fX-fX-fX-mX-fX-mX-mX-fX-mX-mX-
mX-fX-mX-mX-mX-mX-mX-mX-S-mX-S-mX 3′
(Modification key: Table 4 and [ademX-GalNAc] = GalNAc-conjugated nucleotide)

Similar to above, in separate treatment groups, three (3) PLP1 RNAi oligonucleotides (PLP1-2339 sense strand SEQ ID NO: 193, antisense strand SEQ ID NO: 200, PLP1-2398 sense strand SEQ ID NO: 195, antisense strand SEQ ID NO: 202, and PLP1-2340 sense strand SEQ ID NO: 194, antisense strand SEQ ID NO: 201) formulated in PBS were administered by intrathecal injection into the lumbar spine at 30 μg, 100 μg, or 300 μg doses (n=4-5 per treatment group). A control group of mice (n=5) were administered only PBS. After seven (7) days post-injection, mice were sacrificed using CO₂ asphyxiation followed by decapitation. Whole brain and lumbar spinal cord were dissected and preserved for RT-qPCR analysis. RNA was extracted and measured as described in Example 2 to determine PLP1 mRNA levels in the lumbar spinal cord, brain stem, hippocampus and frontal cortex.

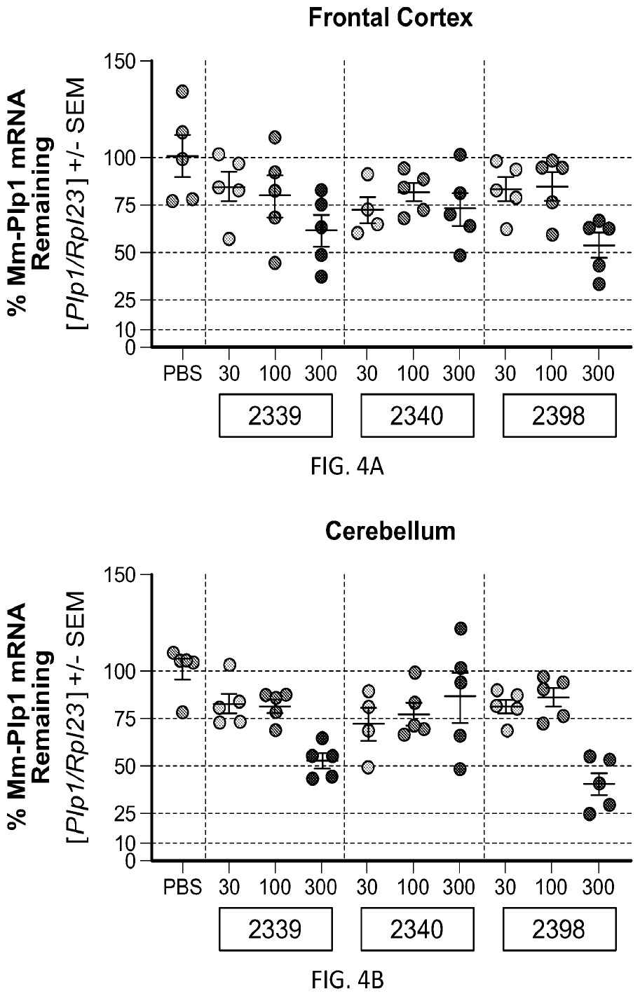

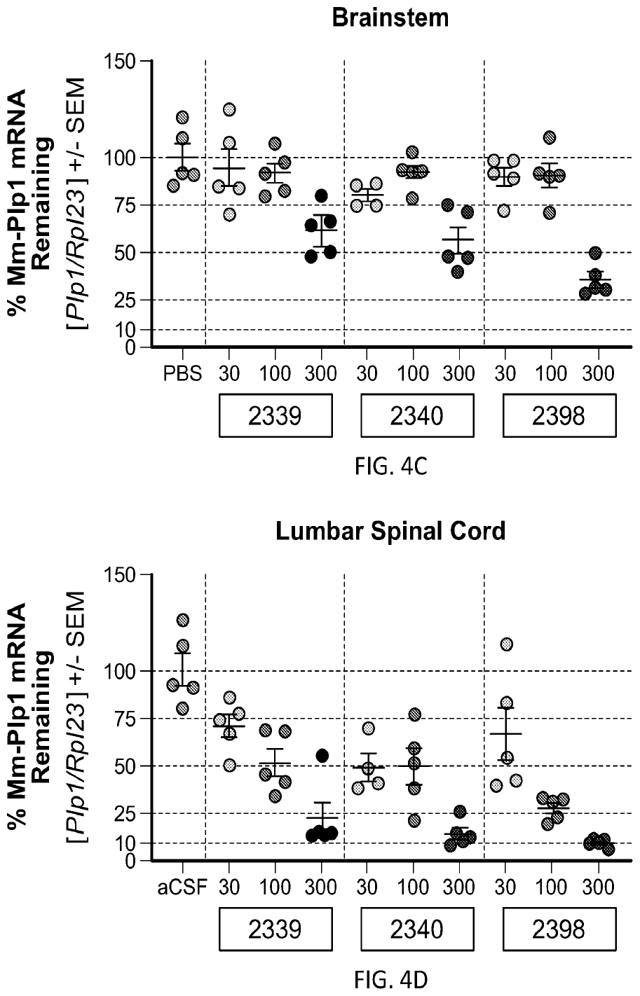

As shown in FIGS. 4A-4D, the indicated PLP1 RNAi oligonucleotides inhibited PLP1 expression in the CNS of mice at regions proximal (e.g., lumbar spinal cord, brain stem; FIGS. 4A-4B) and distal to the site of injection (e.g., hippocampus; frontal cortex; FIGS. 4C-4D), consistent with the results described in FIGS. 2A-2D. As in Example 2, inhibition of PLP1 expression was determined by comparing the percentage of PLP1 mRNA remaining in samples from mice treated with PLP1 RNAi oligonucleotides relative to the percentage of PLP1 mRNA remaining in samples from control mice treated with PBS. These results demonstrate that PLP RNAi oligonucleotides with GalNAc modified tetraloop inhibit PLP1 expression in a dose-dependent manner similar to PLP1 RNAi oligonucleotides with phosphorothioate modified tetraloop in the CNS following intrathecal injection into the lumbar spine.

### Example 4: GalNAc-Conjugated PLP1 RNAi Oligonucleotides Inhibit Human PLP1 Expression in a Mouse Liver Expression Model

To evaluate the ability of PLP1 RNAi oligonucleotides generated by the methods described in Example 1 to inhibit human PLP1 mRNA expression, a hydrodynamic injection (HDI) mouse model was used to transiently express human PLP1 mRNA in the liver (referred to henceforth in the Examples as “HDI mice”). Specifically, eighteen (18) PLP1 RNAi oligonucleotides that target human and monkey PLP1 mRNA were generated (Table 5).

TABLE 5
GalNAc-Conjugated Human/Monkey PLP1 RNAi Oligonucleotides

| | | | SEQ | SEQ ID | SEQ | SEQ ID |
|---|---|---|---|---|---|---|
| | | | ID NO | NO | ID NO | NO |
| Oligo- | | Exon | (Sense) | (Antisense) | (Sense) | (Antisense) |
| nucleotide | DP# | Target | Unmodified | | Modified | |
| PLP1-436 | DP19173P:DP19172G | 3 | 76 | 77 | 112 | 113 |
| PLP1-437 | DP19181P:DP19180G | 3 | 78 | 79 | 114 | 115 |
| PLP1-444 | DP19183P:DP19182G | 3 | 80 | 81 | 116 | 117 |
| PLP1-445 | DP19179P:DP19178G | 3 | 82 | 83 | 118 | 119 |
| PLP1-478 | DP19169P:DP19168G | 4 | 84 | 85 | 120 | 121 |
| PLP1-479 | DP19191P:DP19190G | 4 | 86 | 87 | 122 | 123 |
| PLP1-482 | DP19171P:DP19170G | 4 | 88 | 89 | 124 | 125 |
| PLP1-484 | DP19167P:DP19166G | 4 | 90 | 91 | 126 | 127 |
| PLP1-485 | DP19165P:DP19164G | 4 | 92 | 93 | 128 | 129 |
| PLP1-821 | DP19163P:DP19162G | 5 | 94 | 95 | 130 | 131 |
| PLP1-827 | DP19195P:DP19194G | 5 | 96 | 97 | 132 | 133 |
| PLP1-829 | DP19177P:DP19176G | 5 | 98 | 99 | 134 | 135 |
| PLP1-920 | DP19175P:DP19174G | 6 | 100 | 101 | 136 | 137 |
| PLP1-998 | DP19193P:DP19192G | 7 | 102 | 103 | 138 | 139 |
| PLP1-1011 | DP19185P:DP19184G | 7 | 104 | 105 | 140 | 141 |
| PLP1-1014 | DP19197P:DP19196G | 7 | 106 | 107 | 142 | 143 |
| PLP1-1071 | DP19187P:DP19186G | 8 | 108 | 109 | 144 | 145 |
| PLP1-1075 | DP19189P:DP19188G | 8 | 110 | 111 | 146 | 147 |

The PLP1 RNAi oligonucleotides provided in Table 5 are double-stranded RNAi oligonucleotides comprising a nicked tetraloop GalNAc-conjugated structure having a 36-mer passenger strand and a 22-mer guide strand. The PLP1 mRNA target sequences provided in Table 2 were used to generate the eighteen (18) PLP1 RNAi oligonucleotides that target human and monkey PLP1 mRNA (Table 3). Further, the nucleotide sequences comprising the passenger strand and guide strand have a distinct pattern of modified nucleotides and phosphorothioate linkages (SEQ ID Nos: 112-147). Three of the nucleotides comprising the tetraloop were each conjugated to a GalNAc moiety (CAS #14131-60-3), as described in Example 3 and depicted in FIG. 3.

The GalNAc-conjugated PLP1 RNAi oligonucleotides listed in Table 5 were evaluated in mice engineered to transiently express human PLP1 mRNA in hepatocytes of the mouse liver. Briefly, 6-8-week-old female CD-1 mice (n=5) were subcutaneously administered the indicated GalNAc-conjugated PLP1 RNAi oligonucleotides at a dose of 3 mg/kg formulated in PBS. A control group of mice (n=5) were administered only PBS. Three days later (72 hours), the mice were hydrodynamically injected (HDI) with a DNA plasmid encoding the full human PLP1 gene (25 μg) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine PLP1 mRNA levels as described in Example 2. The values were normalized for transfection efficiency using the NeoR gene included on the DNA plasmid.

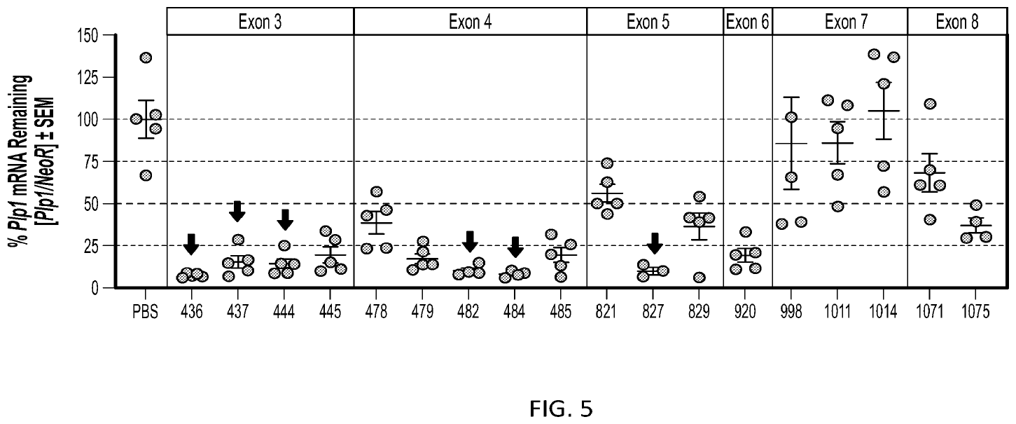

Of the eighteen (18) GalNAc-conjugated PLP1 RNAi oligonucleotides tested, thirteen (13) showed an ED₅₀ below 3 mg/kg (FIG. 5). Potent activity (as determined by >75% PLP1 mRNA silencing, <25% PLP1 mRNA remaining) was observed in ten (10) of the GalNAc-conjugated PLP1 RNAi oligonucleotides tested at 3 mg/kg. These results demonstrate that GalNAc-conjugated PLP1 RNAi oligonucleotides designed to target human PLP1 mRNA inhibited human PLP1 mRNA expression in HDI mice, as determined by a reduction in the amount of human PLP1 mRNA expression in liver samples from HDI mice treated with GalNAc-conjugated PLP1 RNAi oligonucleotides relative to control HDI mice treated with only PBS. As in Example 2, inhibition of PLP1 expression is shown in FIG. 5 by comparing the percentage of PLP1 mRNA remaining in liver samples from HDI mice treated with PLP1 RNAi oligonucleotides to the percentage of PLP1 mRNA remaining in samples from control HDI mice treated with only PBS. Six (6) of the GalNAc-conjugated PLP1 oligonucleotides (PLP-436, PLP1-437, PLP1-444, PLP1-482, PLP1-484 and PLP1-827) were selected for further evaluation.

### Example 5: GalNAc-Conjugated PLP1 RNAi Oligonucleotides Inhibit Human PLP1 Expression in a Dose-Dependent Manner

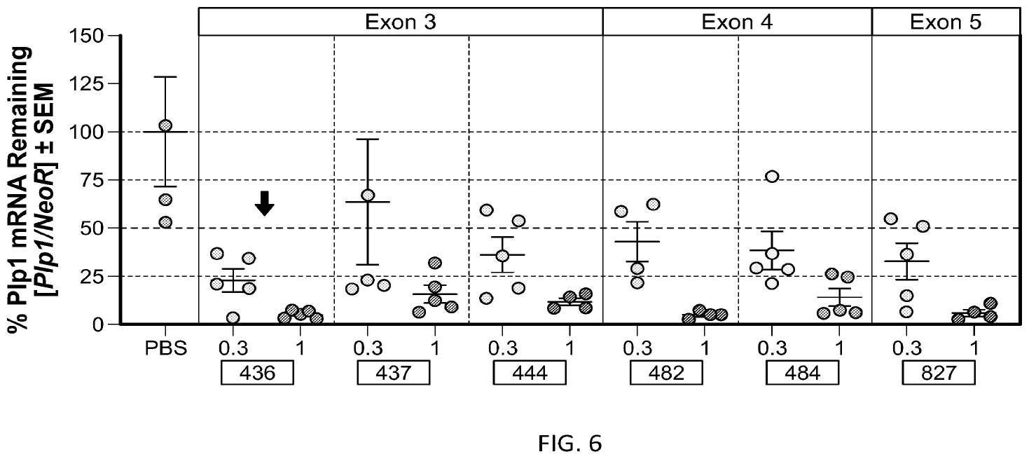

To further evaluate the ability of GalNAc-conjugated PLP1 RNAi oligonucleotides described in Example 4 to inhibit PLP1 expression, mice were treated with GalNAc-conjugated PLP1 RNAi oligonucleotides at two different dose levels. Specifically, in separate treatment groups, GalNAc-conjugated PLP1 RNAi oligonucleotides PLP1-436, PLP1-437, PLP1-444, PLP1-482, PLP1-484, and PLP1-827 formulated in PBS were administered to CD-1 mice as described in Example 4 at dose level of 0.3 mg/kg or 1 mg/kg subcutaneously. A human PLP1 DNA expression plasmid was administered to the mice and liver was collected for qRT-PCR as described in Example 4. As shown in FIG. 6, all of the GalNAc-conjugated PLP1 RNAi oligonucleotides tested inhibited human PLP1 expression in a dose-dependent manner. As in Example 2, inhibition of human PLP1 expression is shown in FIG. 6 by comparing the percentage of PLP1 mRNA remaining in liver samples from HDI mice treated with PLP1 RNAi oligonucleotides to the percentage of PLP1 mRNA remaining in samples from control HDI mice treated with only PBS.

### Example 6: PLP1 RNAi Oligonucleotides Comprising a 2′-O-Methyl Tetraloop Inhibit PLP1 Expression and Demonstrates Tolerability in Mice

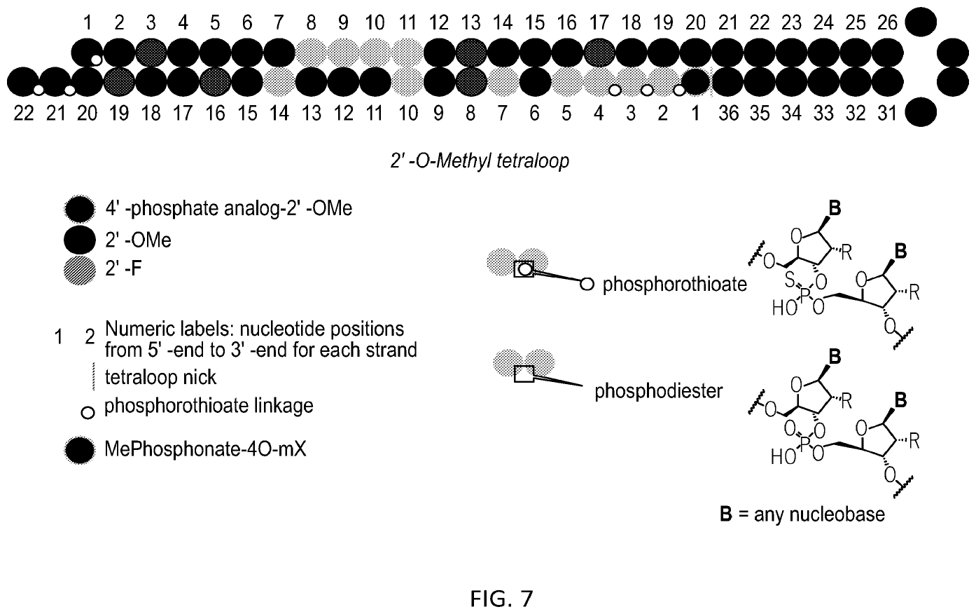

To further evaluate the ability of RNAi oligonucleotides to inhibit PLP1 expression in the central nervous system, an RNAi oligonucleotide targeting PLP1 mRNA with a 2′-O-methyl modified tetraloop was generated. The PLP1-targeting RNAi oligonucleotide was modified such that the tetraloop has 2′-O-methyl modified nucleotides (e.g., see FIG. 7 for a depiction of the generic structure and chemical modification pattern of the modified PLP1 RNAi oligonucleotide). The molecular structure of an RNAi oligonucleotide comprising a 2′-O-methyl tetraloop is illustrated below:

Sense Strand:
5′ mX-S-mX-mX-mX-mX-mX-mX-fX-fX-fX-fX[-mX-]₁₆-mX-mX-mX-mX-
mX-mX-mX-mX-mX 3′
Hybridized to:
Antisense Strand:
5′ [MePhosphonate-40-mX]-S-fX-S-fX-S-fX-fX-mX-fX-mX-mX-fX-mX-
mX-mX-fX-mX-mX-mX-mX-mX-mX-S-mX-S-mX 3′
(Modification key: Table 4)

Mice were treated as described in Example 3 with three groups, aCSF, PLP1-2340 (with the modification pattern depicted in FIG. 3; sense strand SEQ ID NO:194, antisense strand SEQ ID NO: 201), and PLP1-2340 (with the modification pattern depicted in FIG. 7; sense strand SEQ ID NO: 199, antisense SEQ ID NO: 206). Following treatment, on day 7, day 28 and day 56, RNA was extracted from lumbar spinal cord tissue samples to determine PLP1 mRNA levels by qPCR (normalized to endogenous housekeeping gene RPL23). The levels of PLP1 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to PLP1 mRNA. The percentage of PLP1 mRNA remaining in the samples from treated mice was determined using the 2^{-ΔΔCt} (“delta-delta Ct”) method (Livak and Schmittgen (2001) Methods 25:402-08).

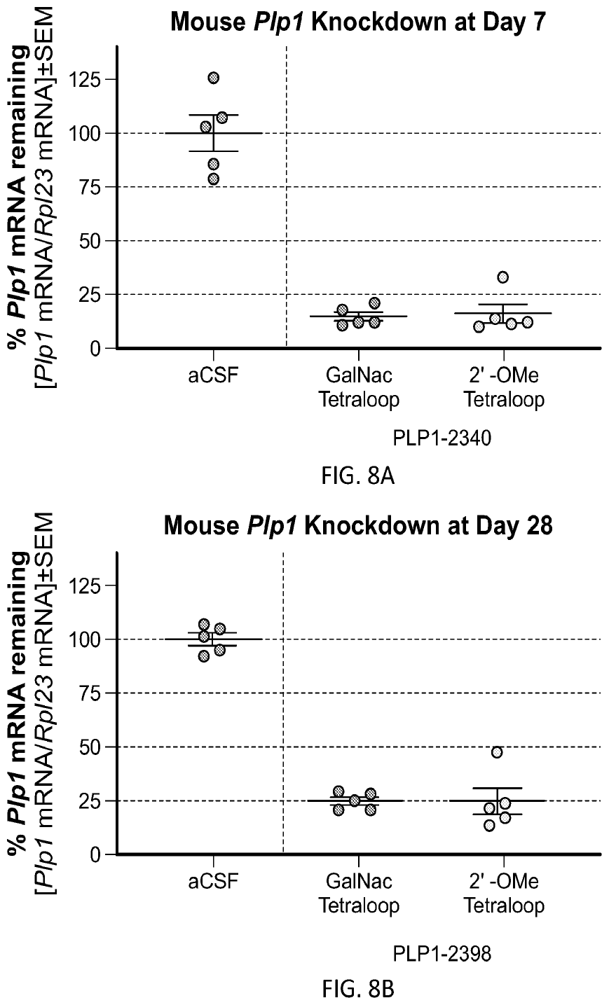

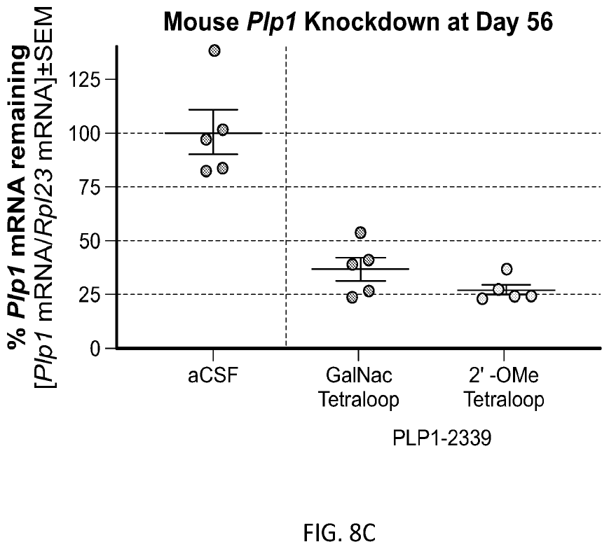

As shown in FIGS. 8A-8C, the indicated PLP1 RNAi oligonucleotides (modified with a GalNAc tetraloop or 2′-OMe tetraloop) inhibited PLP1 expression in the CNS of mice at similar levels.

### Example 7: PLP1 RNAi Oligonucleotides Comprising a 2′-O-Methyl Tetraloop Inhibit PLP1 Expression in Non-Human Primate Central Nervous System

The ability of PLP1 RNAi oligonucleotides comprising a 2′-O-methyl tetraloop (as depicted in FIG. 7) to inhibit PLP1 expression in non-human primates (NHP) was evaluated. The PLP1-targeting RNAi oligonucleotide comprising a 2′-O-methyl modified tetraloop used in this Example has a sense and antisense strand as set forth in SEQ ID NOs: 191 and 192 or 191 and 207, respectively (PLP1-436). The PLP1-targeting RNAi oligonucleotide or control (artificial cerebral spinal fluid (aCSF)) was administered to non-human primates (cynomolgus monkeys) via a single-dose or multi-dose injection into the cisterna magna (i.c.m) (1.5 mL at 30 mg/mL). Animals were given a 45 mg dose on day 0 (single dose) or a 45 mg dose on days 0 and 7 (multi-dose). Twenty-eight (28) days or eighty-four (84) days post-injection, whole brain and lumbar spinal cord were dissected and preserved for RT-qPCR analysis. RNA was extracted from tissue samples from the frontal cortex, parietal cortex, temporal cortex, occipital cortex, cerebellum, brainstem, cervical, thoracic, and lumbar spinal cord, and lumbar dorsal root ganglion to determine PLP1 mRNA levels by qPCR (normalized to endogenous housekeeping genes RPL23 and GAPDH, as indicated). The levels of PLP1 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to PLP1 mRNA. The percentage of PLP1 mRNA remaining in the samples from treated NHPs was determined using the 2^{-ΔΔCt} (“delta-delta Ct”) method (Livak and Schmittgen (2001) Methods 25:402-08).

TABLE 6
Study Design

| | Test | DP | | | | | Dose | Duration |
|---|---|---|---|---|---|---|---|---|
| Cohort | Article | Number | Dose | Volume | ROA | N | on Day | (days) |
| A | aCSF | | — | 1.5 mL | i.c.m. bolus | 3 | 0 | 28 |
| B | GalXC- | DP21591P: | 45 mg | 1.5 mL | i.c.m. bolus | 4 | 0 | 28 |
| | PLP1-436 | DP20254G | | | | | | |
| C | GalXC- | DP21591P: | 45 mg | 1.5 mL | i.c.m. bolus | 4 | 0, 7 | 28 |
| | PLP1-436 | DP20254G | | | | | | |
| D | aCSF | | — | 1.5 mL | i.c.m. bolus | 3 | 0 | 84 |
| E | GalXC- | DP21591P: | 45 mg | 1.5 mL | i.c.m. bolus | 4 | 0 | 84 |
| | PLP1-436 | DP20254G | | | | | | |
| F | GalXC- | DP21591P: | 45 mg | 1.5 mL | i.c.m. bolus | 4 | 0, 7 | 84 |
| | PLP1-436 | DP20254G | | | | | | |

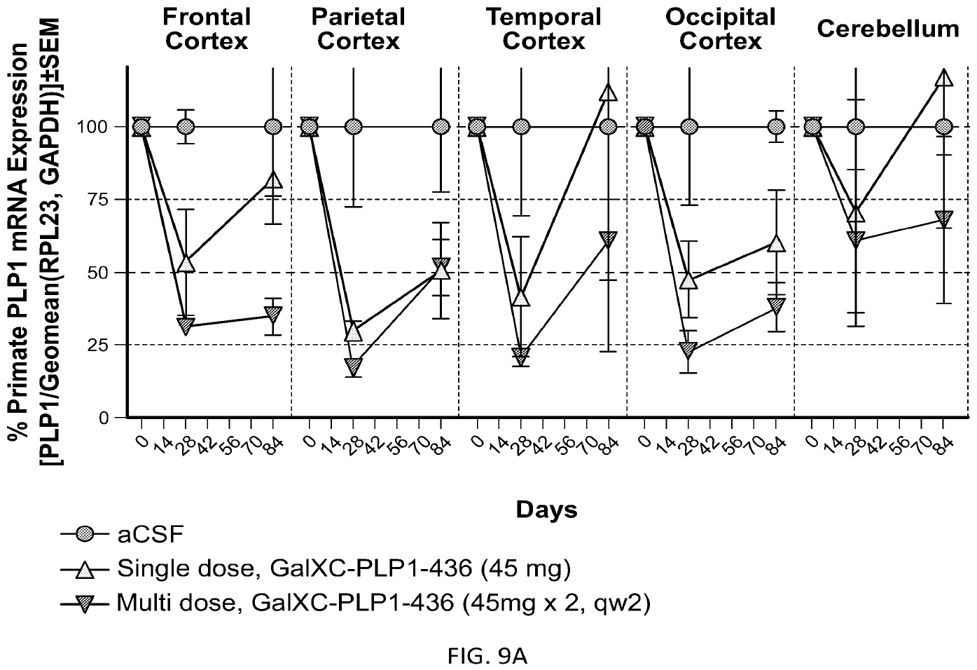

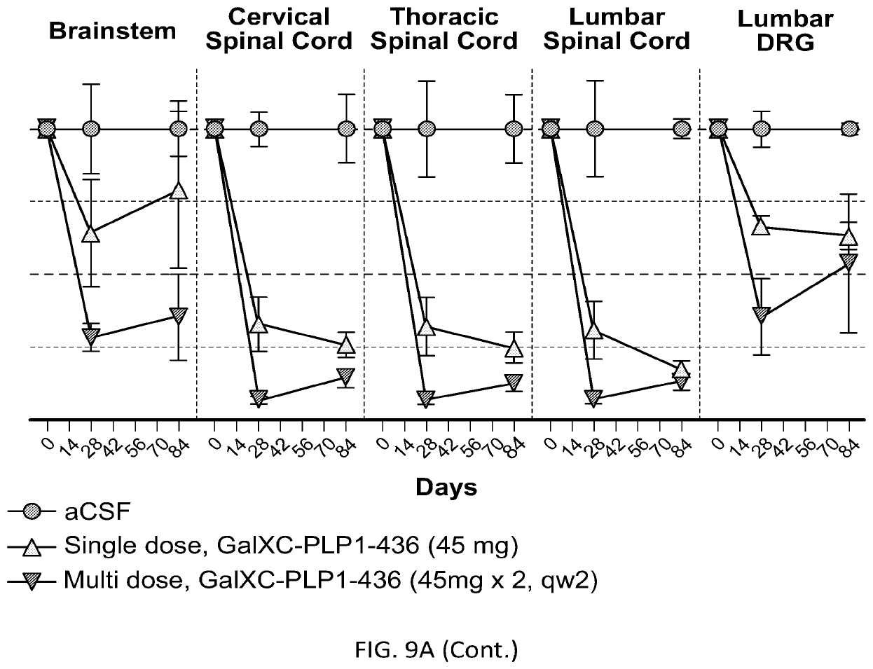

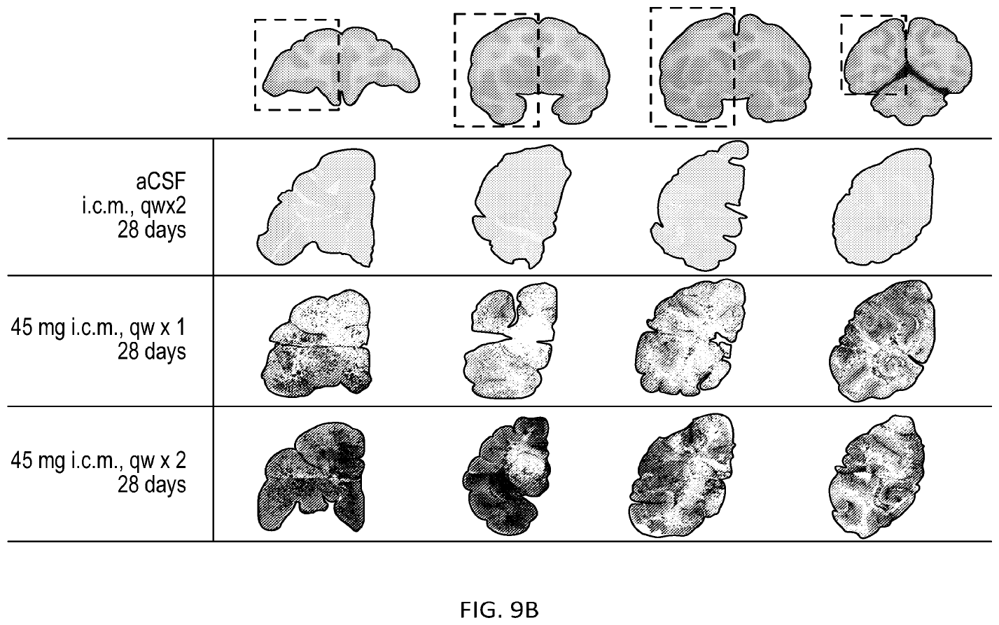

Both the single dose and multi-dose treatments resulted in a reduction in PLP1 mRNA expression in the CNS of NHPs, with increased reduction observed in the multi-dose treatment on days 28 and 84 in multiple brain regions (FIG. 9A). Results of the qPCR analysis were further validated using in situ hybridization labelling of PLP1 mRNA expression in whole brain. Reduction of PLP1 was observed across brain hemispheres, in the cervical spinal cord, and in both grey and white matter (data not shown). Additionally, in situ hybridization of whole brain slices 28-days after single or multi-dose administration of PLP1-436 demonstrated broad distribution of the PLP1 targeting RNAi oligonucleotide across several regions of the brain (FIG. 9B). No adverse clinical observations were seen for either cohort. This study demonstrates that RNAi oligonucleotides targeting PLP1 mRNA comprising a 2′-O-methyl tetraloop and having no targeting ligands reduce PLP1 mRNA expression in the CNS. Further, these results show that a reduction of target gene expression in the CNS is measurable for at least three (3) months following administration of a single or repeated dose, demonstrating the ability of PLP1-targeting RNAi oligonucleotides to provide an extended pharmacodynamic durability in the CNS.

### Example 8: Selection of Reference Dose for Intracerebroventricular Administration to Inhibit PLP1 Expression

To further evaluate the ability of RNAi oligonucleotides to inhibit PLP1 expression in the central nervous system, an RNAi oligonucleotide targeting PLP1 mRNA with a 2′-O-methyl modified tetraloop was generated for intracerebroventricular (i.c.v) administration to mice. The PLP1-targeting RNAi oligonucleotide was modified such that the tetraloop has 2′-O-methyl modified nucleotides as described in Example 6. The PLP1-targeting RNAi oligonucleotide comprising a 2′-O-methyl modified tetraloop used in this Example has an unmodified sense and antisense strand as set forth in SEQ ID NOs: 18 and 19, respectively; and a modified sense and antisense strand as set forth in SEQ ID NOs: 199 and 206, respectively (PLP1-2340). The Plp1-targeting RNAi oligonucleotide or control (artificial cerebral spinal fluid (aCSF)) was administered to 6-week old CD-1 female mice via a single-bolus i.c.v injection at 10 μg, 30 μg, 100 μg, or 300 μg (in 10 μl). Animals were dosed on day 0. Seven days post-injection, whole brain and lumbar spinal cord were dissected and preserved for RT-qPCR analysis. RNA was extracted from tissue samples from the frontal cortex, hippocampus, brainstem, and lumbar spinal cord, to determine Plp1 mRNA levels by qPCR (normalized to endogenous housekeeping gene RPL23). The levels of Pip/mRNA were determined as described in Example 6.

TABLE 7
Study Design

| | | | | | | | Dose | Dur- |
|---|---|---|---|---|---|---|---|---|
| | Test | DP | Dose | Volume | | | on | ation |
| Cohort | Article | Number | (ug) | (uL) | ROA | N | Day | (days) |
| A | aCSF | aCSF | NA | 10 | i.c.v. | 4 | 0 | 7 |
| | | | | | bolus | | | |
| B | 2'OMe | DP19070P: | 10 | 10 | i.c.v. | 4 | 0 | 7 |
| | PLP1 | DP19069G | | | bolus | | | |
| | (2340) | | | | | | | |
| C | 2'OMe | DP19070P: | 30 | 10 | i.c.v. | 4 | 0 | 7 |
| | PLP1 | DP19069G | | | bolus | | | |
| | (2340) | | | | | | | |
| D | 2'OMe | DP19070P: | 100 | 10 | i.c.v. | 4 | 0 | 7 |
| | PLP1 | DP19069G | | | bolus | | | |
| | (2340) | | | | | | | |
| E | 2'OMe | DP19070P: | 300 | 10 | i.v.m. | 4 | 0 | 7 |
| | PLP1 | DP19069G | | | bolus | | | |
| | (2340) | | | | | | | |

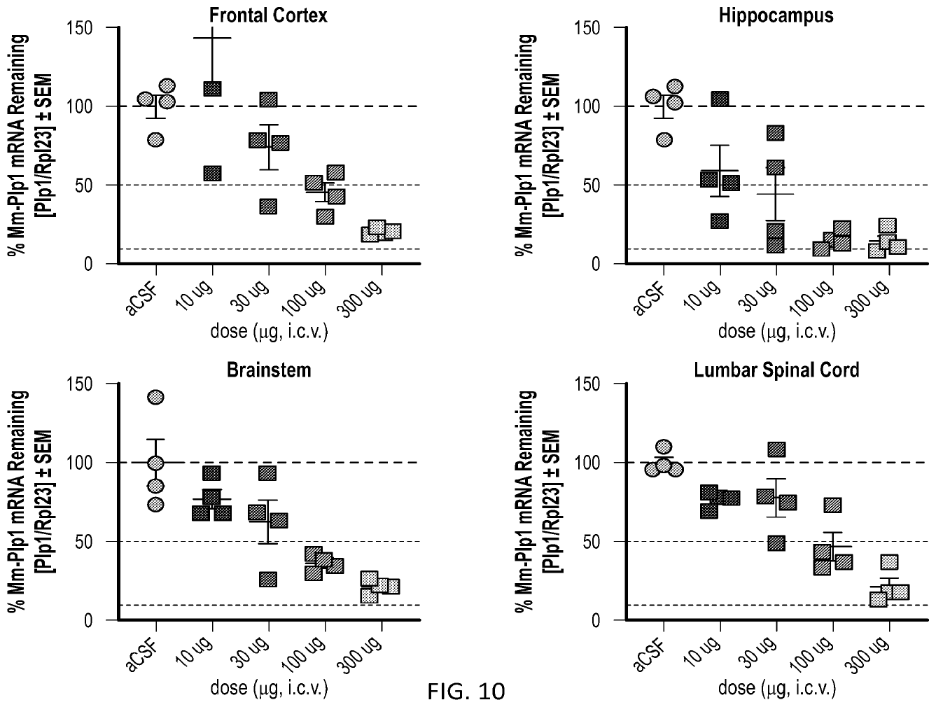

Treatment resulted in a reduction in Plp1 mRNA expression in the CNS of mice with increasing doses of PLP1 RNAi oligonucleotide (FIG. 10). This study demonstrates that a single i.c.v. administration enables reduction of Pip/in the CNS of mice.

### Example 9: PLP1 RNAi Oligonucleotides Comprising a 2′-O-Methyl Tetraloop Inhibit Mouse Plp1 Expression in a Dose-Dependent Manner in Plp1-Dup Mice

Mutations leading to PLP1 duplication are common in human patients with Pelizaeus-Merzbacher disease. To assess the efficiency of Plp1 RNAi oligonucleotides to reduce Plp1 expression in the presence of increased expression (i.e. duplication) of Plp1, Plp1-dup mice were used (Clark et al. (2013) The Journal of Neuroscience, 33(29): 11788-99). Plp1-dup are known to express upwards of a 1000% increase in Plp1 compared to wild type mice. To establish an effective dose for treatment, the PLP1-targeting RNAi oligonucleotide (described in Example 8; PLP1-2340 with a sense and antisense strand of SEQ ID NOs: 199 and 206, respectively) or control (artificial cerebral spinal fluid (aCSF)) was administered to 11-12 week old C57B/L or Plp1-dup male mice via a single-bolus i.c.v injection at 30 μg, 100 μg, 300 μg, or 500 μg (in 10 μl). Animals were dosed on day 0, and seven days post-injection, whole brain and lumbar spinal cord were dissected and preserved for RT-qPCR analysis. RNA was extracted from tissue samples from the frontal cortex, hippocampus, brainstem, somatosensory cortex, cerebellum, and lumbar spinal cord, to determine Plp1 mRNA levels by qPCR (normalized to endogenous housekeeping gene RPL23). The levels of Pip/mRNA were determined as described in Example 6.

TABLE 8
Study Design

| | | | | | | | Dur- |
|---|---|---|---|---|---|---|---|
| | Test | DP | Dose | | | | ation |
| Cohort | Article | Number | (ug) | Strain | ROA | N | (days) |
| A | aCSF | N/A | NA | C57BL/6 | i.c.v. bolus | 8 | 7 |
| B | aCSF | N/A | NA | Plp1-dup | i.c.v. bolus | 8 | |
| C | 2'OMe | DP19070P: | 30 | C57BL/6 | i.c.v. bolus | 8 | 7 |
| | PLP1 | DP19069G | | | | | |
| | (2340) | | | | | | |
| D | 2'OMe | DP19070P: | 100 | C57BL/6 | i.c.v. bolus | 8 | 7 |
| | PLP1 | DP19069G | | | | | |
| | (2340) | | | | | | |
| E | 2'OMe | DP19070P: | 300 | C57BL/6 | i.c.v. bolus | 8 | 7 |
| | PLP1 | DP19069G | | | | | |
| | (2340) | | | | | | |
| F | 2'OMe | DP19070P: | 500 | C57BL/6 | i.c.v. bolus | 8 | 7 |
| | PLP1 | DP19069G | | | | | |
| | (2340) | | | | | | |
| G | aCSF | N/A | NA | C57BL/6 | i.c.v. bolus | 8 | 7 |
| H | aCSF | N/A | NA | Plp1-dup | i.c.v. bolus | 8 | 7 |
| I | 2'OMe | DP19070P: | 30 | Plp1-dup | i.c.v. bolus | 8 | 7 |
| | PLP1 | DP19069G | | | | | |
| | (2340) | | | | | | |
| J | 2'OMe | DP19070P: | 100 | Plp1-dup | i.c.v. bolus | 8 | 7 |
| | PLP1 | DP19069G | | | | | |
| | (2340) | | | | | | |
| K | 2'OMe | DP19070P: | 300 | Plp1-dup | i.c.v. bolus | 8 | 7 |
| | PLP1 | DP19069G | | | | | |
| | (2340) | | | | | | |
| L | 2'OMe | DP19070P: | 500 | Plp1-dup | i.c.v. bolus | 8 | 7 |
| | PLP1 | DP19069G | | | | | |
| | (2340) | | | | | | |

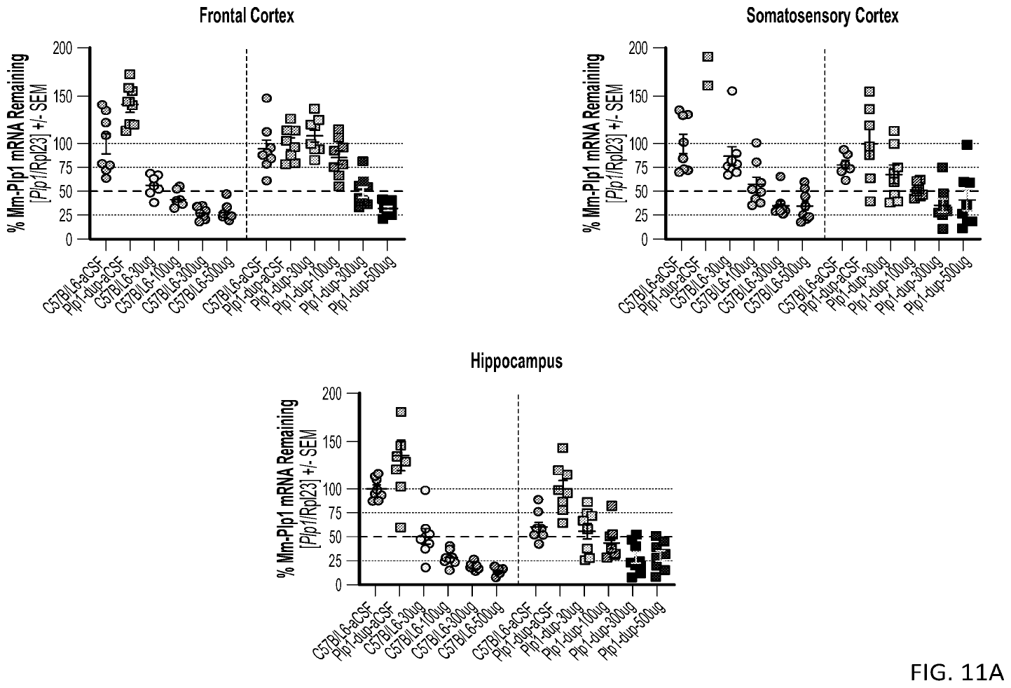

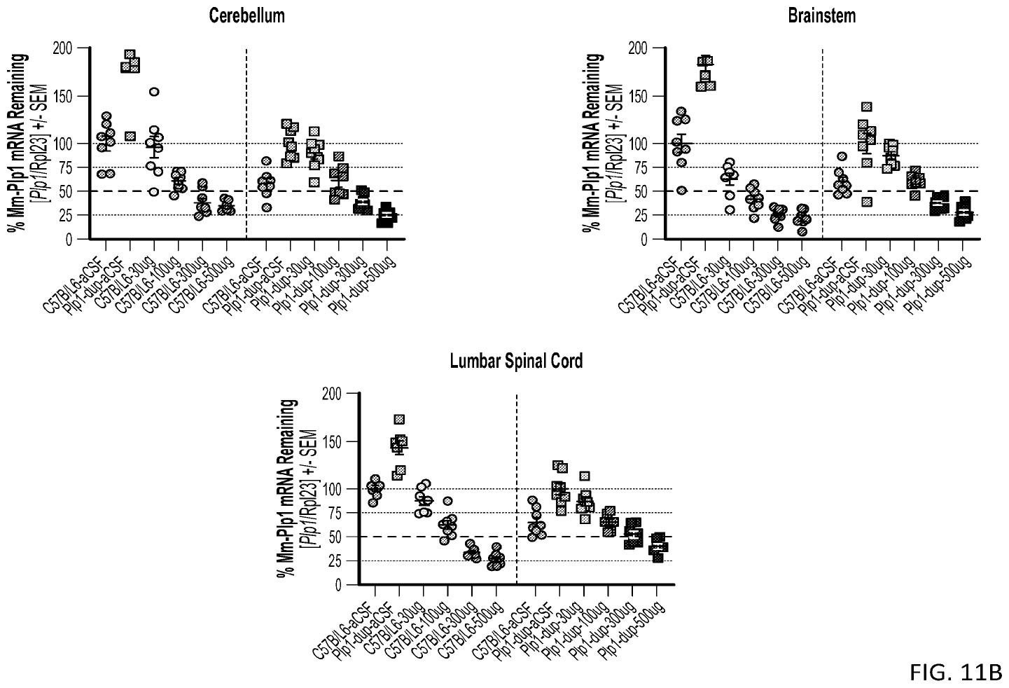

Treatment resulted in a reduction in Plp1 mRNA expression in the CNS of both wild-type (C57B/L) and Plp1-dup mice with increasing doses of PLP1 RNAi oligonucleotide (FIGS. 11A and 11B). This study demonstrates that a single i.c.v. administration enables reduction of Plp1 in the CNS. Specifically, potent knock-down is observed in mice expressing a Plp1 duplication demonstrating the efficiency of the Plp1 RNAi oligonucleotide to reduce Plp1 expression. Notably, at some doses Plp1 is not knocked down below wild-type levels as some expression of Plp1 is needed in the CNS for proper brain function.

### Example 10: PLP1 RNAi Oligonucleotides Comprising a 2′-O-Methyl Tetraloop Inhibit Plp1 Expression and Reduces Astrogliosis

Astrogliosis is a response in the central nervous system due to damage after injury (e.g., a neurodegenerative disease such as Pelizaeus-Merzbacher disease). Through this process, astrocytes change their molecular expression and morphology including but not limited to increased proliferation and hypertrophy. A known marker of astrogliosis is an increase in glial fibrillary acid protein (GFAP). As an intermediate filament, GFAP maintains cell cytoarchitecture and mechanical strength of astrocytes thereby providing stability for the astrocyte response to damage.

As expected, Plp1-dup mice express higher levels of Plp1 mRNA and Plp1 protein throughout the brain when compared to wild-type mice (data not shown). As Plp1 is exclusively expressed in oligodendrocytes, the number of oligodendrocytes in the Plp1-dup mice was investigated. The increase in Plp1 did not appear to be due to an increase in oligodendrocytes (data not shown). However, with increased Plp1, apparent myelin degeneration and morphological disruption is observed in the corpus callosum of 91-day old mice. Additionally, profound astrocyte activation (as measured through GFAP expression) is observed throughout the CNS (e.g., in the brainstem and spinal cord grey matter) (data not shown). This data demonstrates increased astrogliosis (i.e. increased GFAP) in mice expressing high levels of Plp1.

The ability of Plp1 RNAi oligonucleotides to reduce Plp1 expression, and ultimately reduce GFAP expression over time, was assessed. Specifically, the PLP1-targeting RNAi oligonucleotide (described in Example 8; PLP1-2340 with a sense and antisense strand of SEQ ID NOs: 199 and 206, respectively) or control (artificial cerebral spinal fluid (aCSF)) was administered to 11-12 week old C57B/L or Plp1-dup male mice via a single-bolus i.c.v injection at 500 μg. Animals were dosed on day 0. Whole brain and lumbar spinal cord were dissected on days 7, 14, 28, 56, and 84 post-injection from a cohort of mice and preserved for RT-qPCR analysis. RNA was extracted from tissue samples from the frontal cortex, hippocampus, brainstem, cerebellum, and lumbar spinal cord, to determine Plp1 mRNA levels by qPCR (normalized to endogenous housekeeping gene RPL23). The levels of Plp1 mRNA were determined as described in Example 6.

TABLE 9
Study Design

| Dose | Dose | | | | Duration | |
|---|---|---|---|---|---|---|
| (ug) | (ug) | Strain | ROA | Test Article | (weeks) | N |
| A | N/A | C57B1/6 | ICV | aCSF | 1 | 9 |
| B | N/A | Plpl-dup | ICV | aCSF | 1 | 9 |
| C | 500 | Plp1-dup | ICV | PLP1-2340 | 1 | 9 |
| D | N/A | C57B1/6 | ICV | aCSF | 2 | 9 |
| E | N/A | Plp1-dup | ICV | aCSF | 2 | 9 |
| F | 500 | Plpl-dup | ICV | PLP1-2340 | 2 | 9 |
| G | N/A | C57B1/6 | ICV | aCSF | 4 | 9 |
| H | N/A | Plp1-dup | ICV | aCSF | 4 | 9 |
| I | 500 | Plp1-dup | ICV | PLP1-2340 | 4 | 9 |
| J | N/A | C57B1/6 | ICV | aCSF | 8 | 9 |
| K | N/A | Plpl-dup | ICV | aCSF | 8 | 9 |
| L | 500 | Plp1-dup | ICV | PLP1-2340 | 8 | 9 |
| M | N/A | C57B1/6 | ICV | aCSF | 12 | 9 |
| N | N/A | Plp1-dup | ICV | aCSF | 12 | 9 |
| O | 500 | Plp1-dup | ICV | PLP1-2340 | 12 | 9 |
| P | N/A | C57B1/6 | ICV | aCSF | 16 | 9 |
| Q | N/A | Plp1-dup | ICV | aCSF | 16 | 9 |
| R | 500 | Plp1-dup | PLP1-2340 | PLP1-2340 | 16 | 9 |

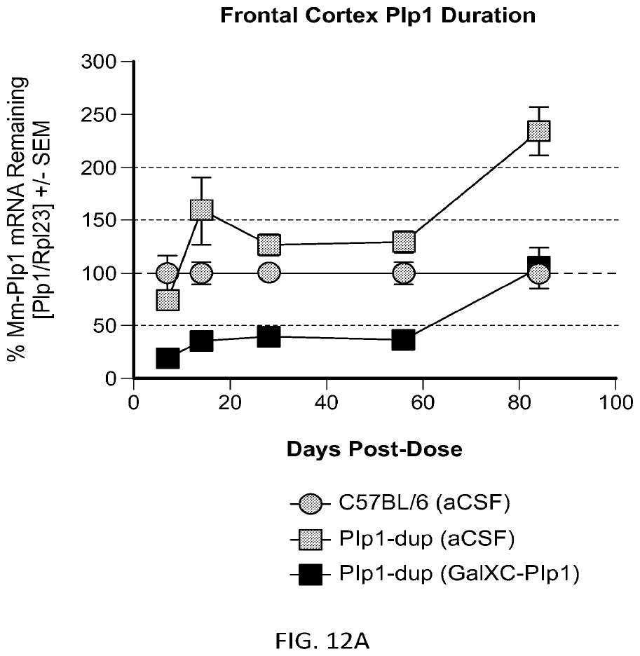

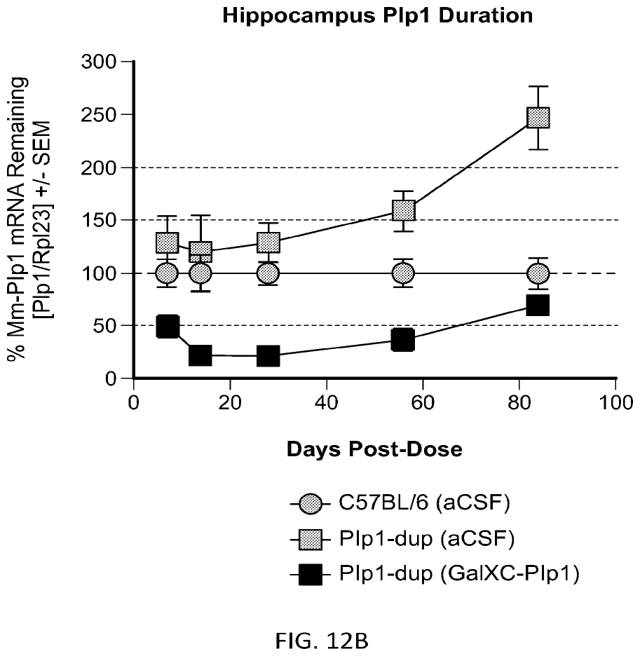

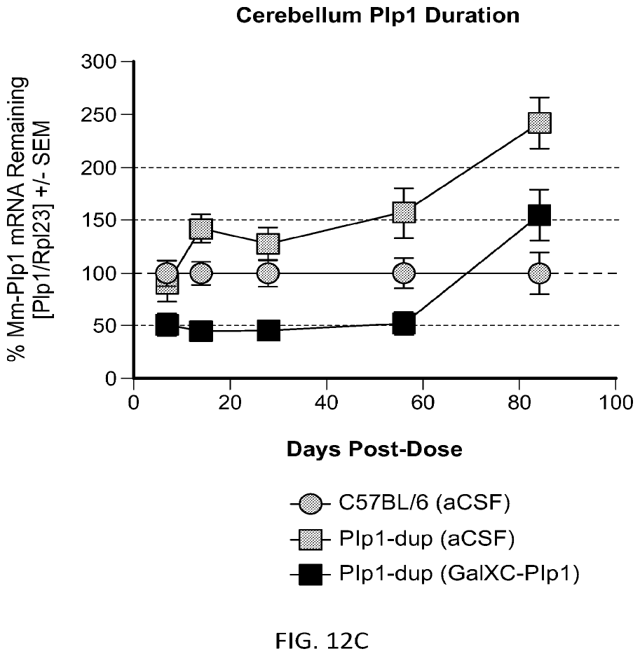

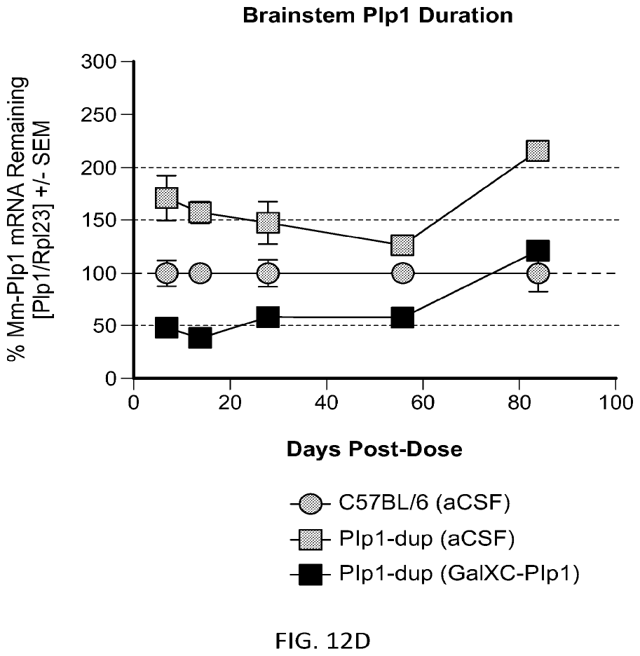

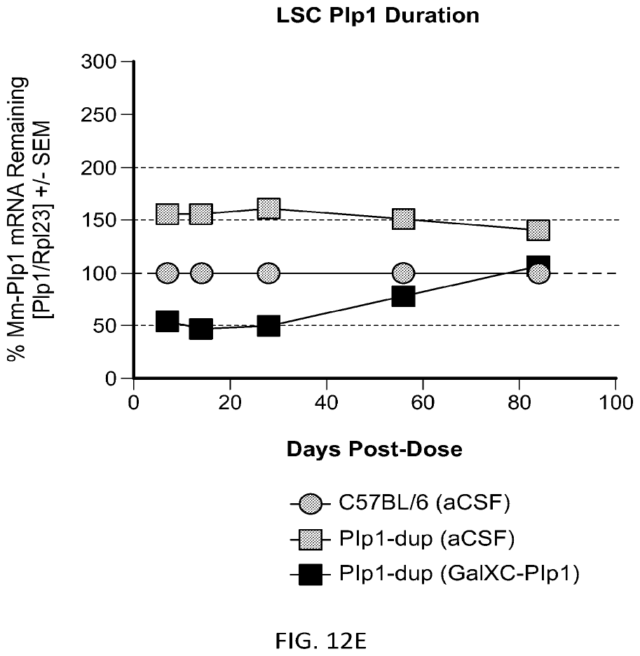

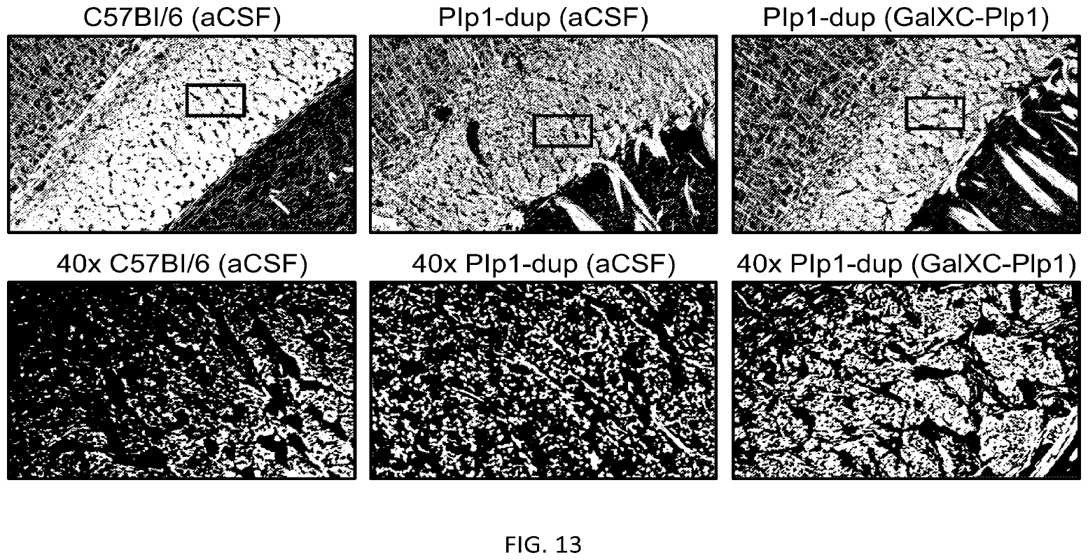

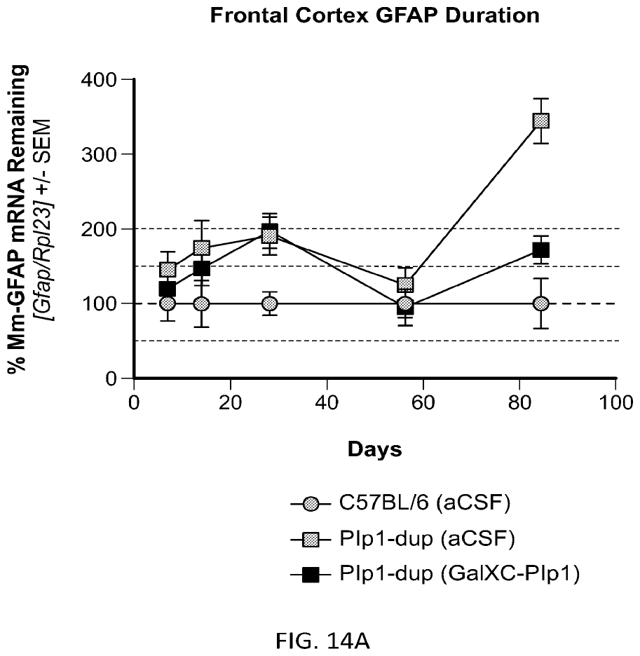

Treatment resulted in a reduction in Plp1 mRNA expression in the CNS (frontal cortex, hippocampus, cerebellum, brainstem, and lumbar spinal cord) of Plp1-dup mice (FIGS. 12A-12E). Specifically, up to 75% Plp1 silence was observed at day 7, greater than 80% at day 14, up to 85% at day 28, and up to 75% silencing was observed through day 56 and 84. Reduced PLP1 protein expression in the corpus callosum was also observed (FIG. 13). This study demonstrates that a single i.c.v. administration enables sustained reduction of Plp1 in the CNS over time. Specifically, potent knock-down is observed in mice expressing a Plp1 duplication demonstrating the efficiency of the Plp1 RNAi oligonucleotide to reduce Plp1 expression.

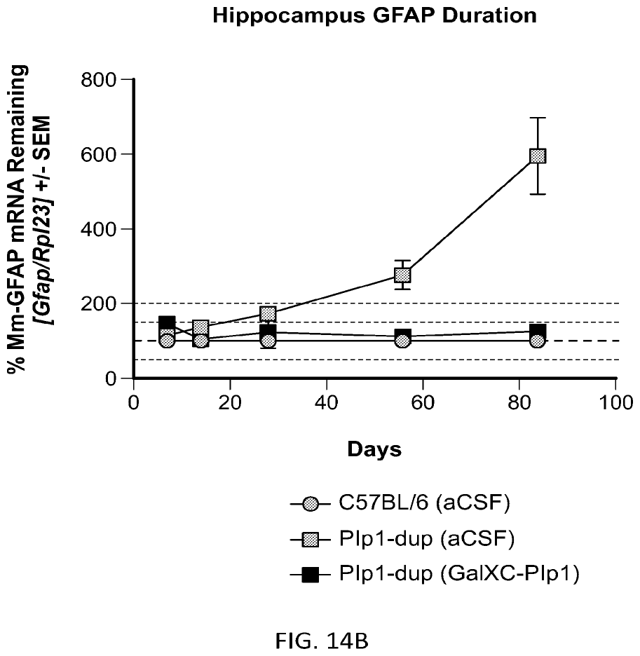

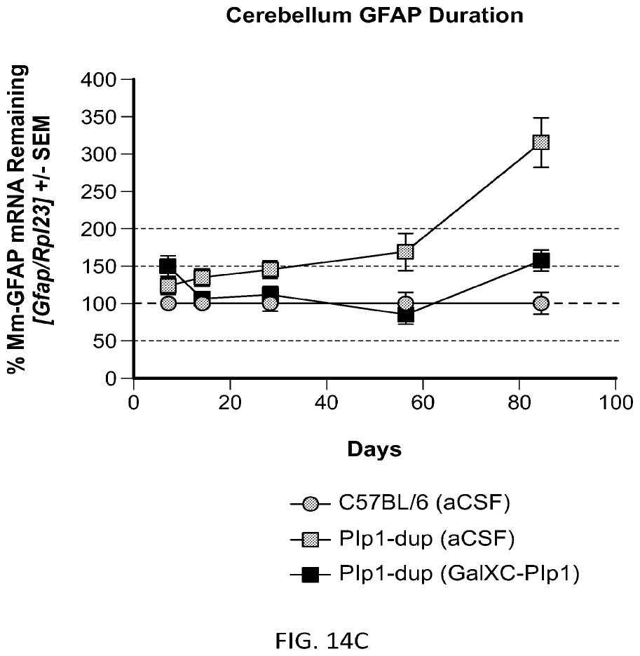

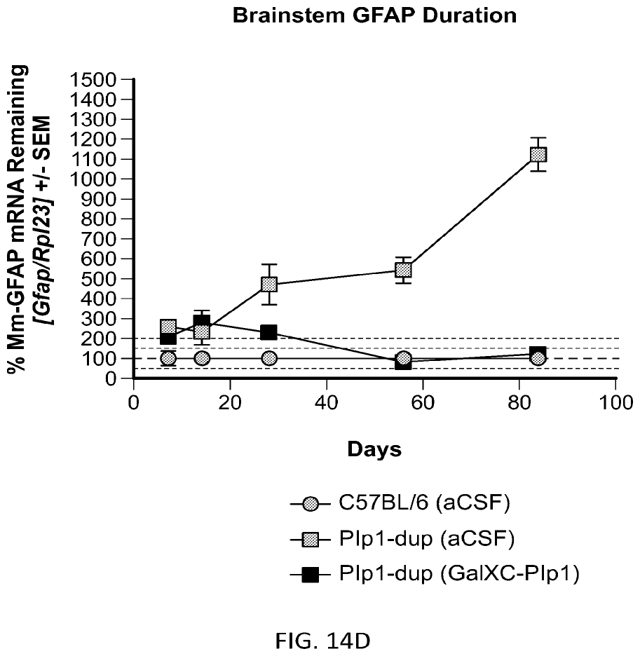

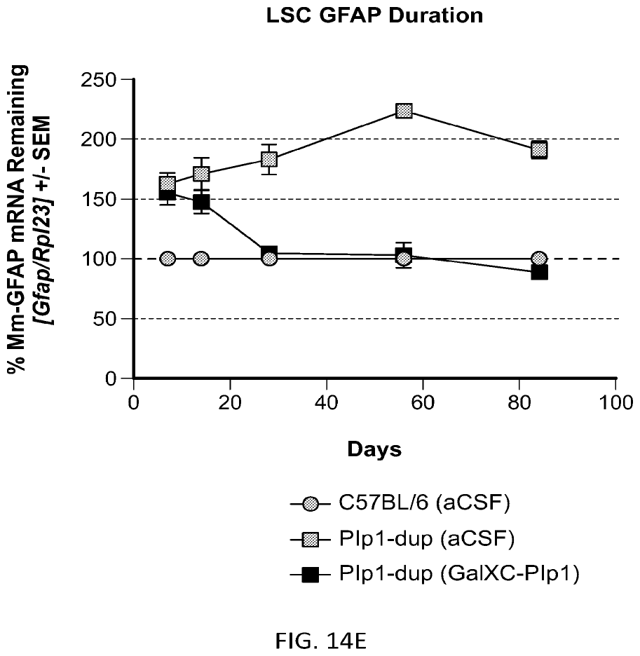

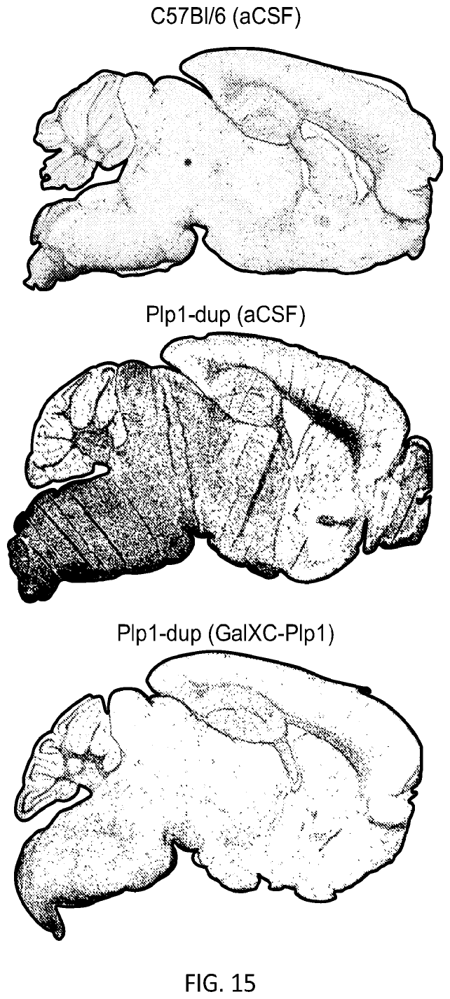

Treatment with the Plp1 RNAi oligonucleotide also resulted in reduction of Gfap expression. When compared to C57BL/6, Plp1-dup mice have up to about 1100% overexpression of Gfap. Following administration with PLP1-2340, Gfap expression was reduced down to wild-type levels (FIGS. 14A-14E). As early as day 7, a reduction was observed in some brain regions (e.g. brain stem), whereas reduction in other regions (e.g., hippocampus and cerebellum) were observed as early as day 14. The reduction in Gfap expression and ultimately Gfap protein in the brain (as measured by immunofluorescence of whole brain slices; FIG. 15), corresponded with a reduction/reversal of astrogliosis and dysmyelination in the corpus callosum at least 56 days after administration of the Plp1 RNAi oligonucleotide. This study demonstrates that a single i.c.v administration of PLP1-targeting RNAi oligonucleotide reduces Gfap expression and astrogliosis and dysmyelination.

### Example 11: PLP1 RNAi Oligonucleotides Comprising a 2′-O-Methyl Tetraloop Inhibit Mouse Plp1 Expression in a Dose-Dependent Manner in Neonatal Mice

To determine a potent dose for treating neonatal mice, the PLP1-targeting RNAi oligonucleotide (described in Example 8; PLP1-2340 with a sense and antisense strand of SEQ ID NOs: 199 and 206, respectively) or control (artificial cerebral spinal fluid (aCSF)) was administered to age P4 C57BL/6 male mice via a single-bolus i.c.v injection at 10 μg, 30 μg, 100 μg, or 250 μg. Animals were dosed on day 0, and seven days post-injection, whole brain and spinal cord were dissected and preserved for RT-qPCR analysis. RNA was extracted from the left hemisphere, right hemisphere, and spinal cord, to determine Plp1, Gfap, and Mbp (myelin basic protein) mRNA levels by qPCR (normalized to endogenous housekeeping gene RPL23). The levels of mRNA were determined as described in Example 6.

TABLE 10
Study Design

| | Dose | | | | | | Duration |
|---|---|---|---|---|---|---|---|
| Cohort | (ug) | Strain | ROA | Age | Test Article | N | (days) |
| A | N/A | C57B/L | ICV | P4 | aCSF | 9 | 7 |
| B | 10 | C57B/L | ICV | P4 | PLP1-2340 | 9 | 7 |
| C | 30 | C57B/L | ICV | P4 | PLP1-2340 | 9 | 7 |
| D | 100 | C57B/L | ICV | P4 | PLP1-2340 | 9 | 7 |
| E | 250 | C57B/L | ICV | P4 | PLP1-2340 | 9 | 7 |

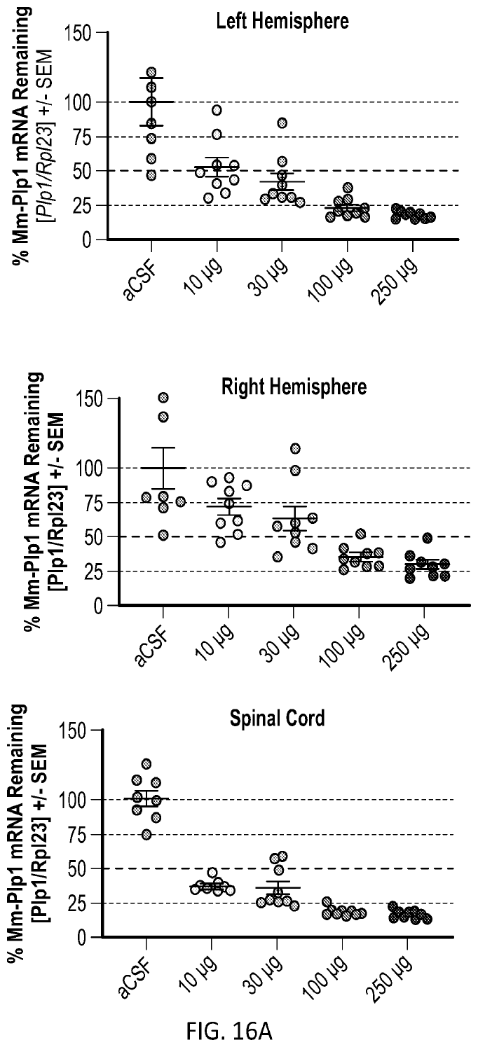

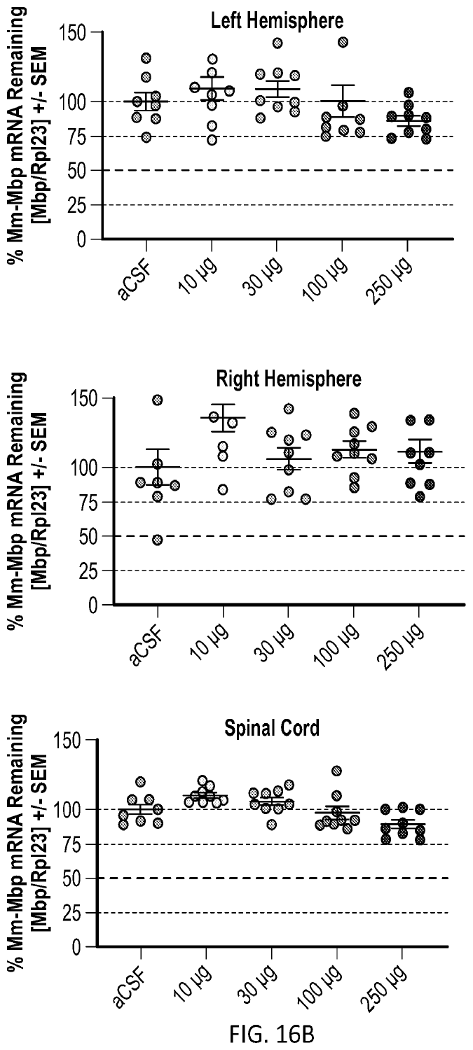

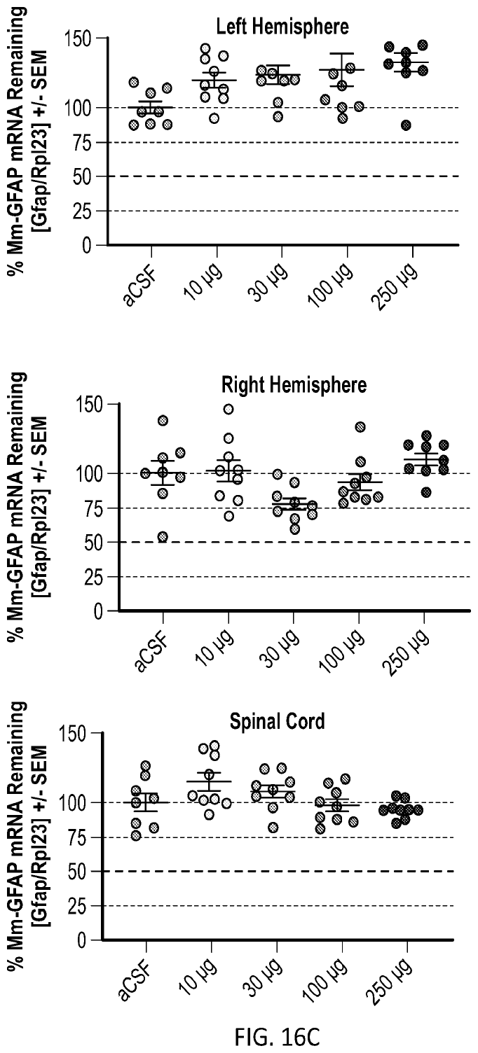

Treatment resulted in a reduction in Plp1 mRNA expression in each brain region with increasing doses of Plp1 RNAi oligonucleotide administered at P4 (FIG. 16A). However, no difference was observed for Mbp or Gfap expression (FIGS. 16B and 16C, respectively) in mice administered at P4, indicating astrogliosis nor oligodendrocyte death was inadvertently induced. This study demonstrates that a single i.c.v. administration enables reduction of Plp1 in the CNS of neonatal mice.

### Example 12: PLP1 RNAi Oligonucleotides Comprising a 2′-O-Methyl Tetraloop Inhibit Mouse Plp1 Expression in Early Intervention for Neonatal Mice

Diseases associated with aberrant PLP1 expression often affect children. To assess the ability of PLP1-targeting RNAi oligonucleotides to act as early intervention for treatment, the PLP1-targeting RNAi oligonucleotide (described in Example 8; PLP1-2340 with a sense and antisense strand of SEQ ID NOs: 199 and 206, respectively) or control (artificial cerebral spinal fluid (aCSF)) was administered to P4 C57Bl/6 and Plp1-dup male mice via a single-bolus i.c.v injection at 250 μg. Animals were dosed on day 0, and 24 days post-injection (age P28) whole brain and spinal cord were dissected and preserved for RT-qPCR analysis. RNA was extracted from the frontal cortex, hippocampus, cerebellum, brain stem, and lumbar spinal cord, to determine Plp1, Gfap, and Mbp mRNA levels by qPCR (normalized to endogenous housekeeping gene RPL23). The levels of mRNA were determined as described in Example 6.

TABLE 11
Study Design

| | Dose | | | | | | Duration |
|---|---|---|---|---|---|---|---|
| Cohort | (ug) | Strain | ROA | Age | Test Article | N | (days) |
| A | N/A | C57B/L | ICV | P4 | aCSF | 10 | 24 (P28) |
| B | N/A | Plp1-dup | ICV | P4 | aCSF | 10 | 24 (P28) |
| C | 250 | Plp1-dup | ICV | P4 | PLP1-2340 | 10 | 24 (P28) |

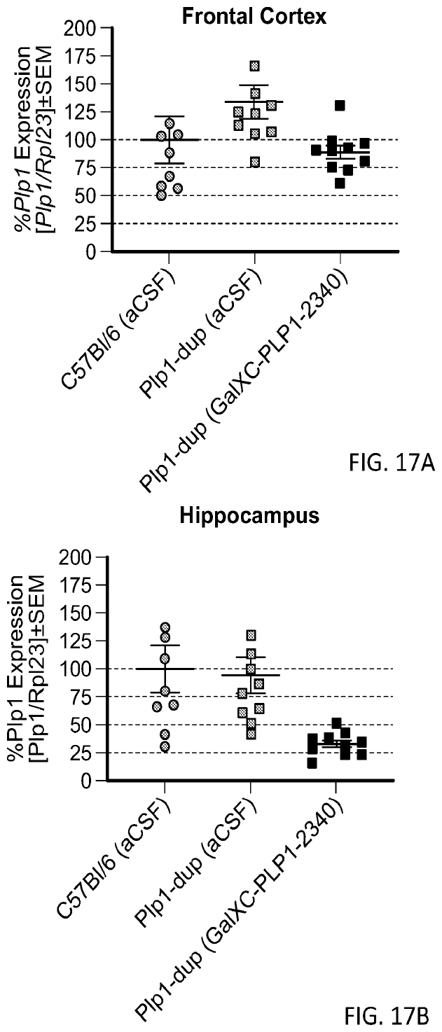

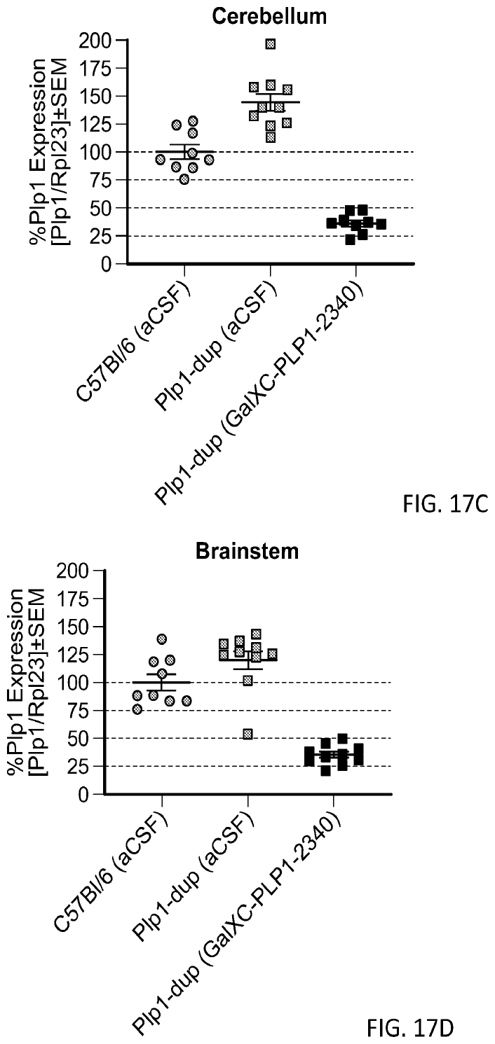

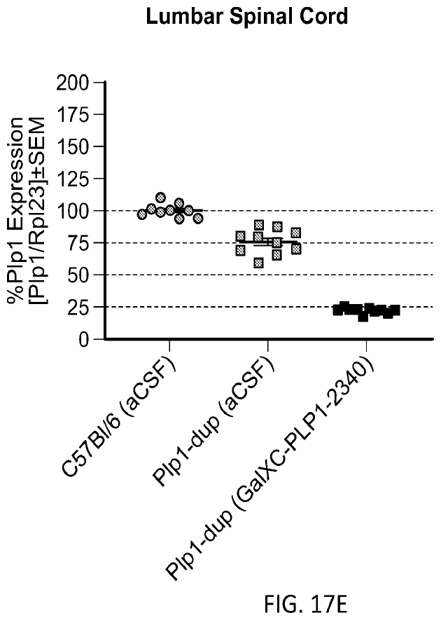

Treatment resulted in a reduction in Plp1 mRNA expression in each brain region (FIGS. 17A-17E). This study demonstrates that a single neonatal i.c.v. administration reduces Pip/expression in the CNS of neonatal mice.

Sequence Listing

| | | | SEQ ID |
|---|---|---|---|
| Name | Description | Sequence | NO |
| Human | human | ACUUUCAUGGCUUCUCACGCUUGUGCUGCAUA | 1 |
| (Hs) | PLP1 | UCCCACACCAAUUAGACCCAAGGAUCAGUUGG | |
| | mRNA | AAGUUUCCAGGACAUCUUCAUUUUAUUUCCAC | |
| | NM_001128834.2 | CCUCAAUCCACAUUUCCAGAUGUCUCUGCAGC | |
| | (GenBank | AAAGCGAAAUUCCAGGAGAAGAGGACAAAGA | |
| | Ref#) | UACUCAGAGAGAAAAAGUAAAAGACCGAAGA | |
| | | AGGAGGCUGGAGAGACCAGGAUCCUUCCAGCU | |
| | | GAACAAAGUCAGCCACAAAGCAGACUAGCCAG | |
| | | CCGGCUACAAUUGGAGUCAGAGUCCCAAAGAC | |
| | | AUGGGCUUGUUAGAGUGCUGUGCAAGAUGUC | |
| | | UGGUAGGGGCCCCCUUUGCUUCCCUGGUGGCC | |
| | | ACUGGAUUGUGUUUCUUUGGGGUGGCACUGU | |
| | | UCUGUGGCUGUGGACAUGAAGCCCUCACUGGC | |
| | | ACAGAAAAGCUAAUUGAGACCUAUUUCUCCAA | |
| | | AAACUACCAAGACUAUGAGUAUCUCAUCAAUG | |
| | | UGAUCCAUGCCUUCCAGUAUGUCAUCUAUGGA | |
| | | ACUGCCUCUUUCUUCUUCCUUUAUGGGGCCCU | |
| | | CCUGCUGGCUGAGGGCUUCUACACCACCGGCG | |
| | | CAGUCAGGCAGAUCUUUGGCGACUACAAGACC | |
| | | ACCAUCUGCGGCAAGGGCCUGAGCGCAACGGU | |
| | | AACAGGGGGCCAGAAGGGGAGGGGUUCCAGA | |
| | | GGCCAACAUCAAGCUCAUUCUUUGGAGCGGGU | |
| | | GUGUCAUUGUUUGGGAAAAUGGCUAGGACAU | |
| | | CCCGACAAGUUUGUGGGCAUCACCUAUGCCCU | |
| | | GACCGUUGUGUGGCUCCUGGUGUUUGCCUGCU | |
| | | CUGCUGUGCCUGUGUACAUUUACUUCAACACC | |
| | | UGGACCACCUGCCAGUCUAUUGCCUUCCCCAG | |
| | | CAAGACCUCUGCCAGUAUAGGCAGUCUCUGUG | |
| | | CUGAUGCCAGAAUGUAUGGUGUUCUCCCAUGG | |
| | | AAUGCUUUCCCUGGCAAGGUUUGUGGCUCCAA | |
| | | CCUUCUGUCCAUCUGCAAAACAGCUGAGUUCC | |
| | | AAAUGACCUUCCACCUGUUUAUUGCUGCAUUU | |
| | | GUGGGGGCUGCAGCUACACUGGUUUCCCUGCU | |
| | | CACCUUCAUGAUUGCUGCCACUUACAACUUUG | |
| | | CCGUCCUUAAACUCAUGGGCCGAGGCACCAAG | |
| | | UUCUGAUCCCCCGUAGAAAUCCCCCUUUCUCU | |
| | | AAUAGCGAGGCUCUAACCACACAGCCUACAAU | |
| | | GCUGCGUCUCCCAUCUUAACUCUUUGCCUUUG | |
| | | CCACCAACUGGCCCUCUUCUUACUUGAUGAGU | |
| | | GUAACAAGAAAGGAGAGUCUUGCAGUGAUUA | |
| | | AGGUCUCUCUUUGGACUCUCCCCUCUUAUGUA | |
| | | CCUCUUUUAGUCAUUUUGCUUCAUAGCUGGUU | |
| | | CCUGCUAGAAAUGGGAAAUGCCUAAGAAGAU | |
| | | GACUUCCCAACUGCAAGUCACAAAGGAAUGGA | |
| | | GGCUCUAAUUGAAUUUUCAAGCAUCUCCUGAG | |
| | | GAUCAGAAAGUAAUUUCUUCUCAAAGGGUAC | |
| | | UUCCACUGAUGGAAACAAAGUGGAAGGAAAG | |
| | | AUGCUCAGGUACAGAGAAGGAAUGUCUUUGG | |
| | | UCCUCUUGCCAUCUAUAGGGGCCAAAUAUAUU | |
| | | CUCUUUGGUGUACAAAAUGGAAUUCAUUCUG | |
| | | GUCUCUCUAUUACCACUGAAGAUAGAAGAAA | |
| | | AAAGAAUGUCAGAAAAACAAUAAGAGCGUUU | |
| | | GCCCAAAUCUGCCUAUUGCAGCUGGGAGAAGG | |
| | | GGGUCAAAGCAAGGAUCUUUCACCCACAGAAA | |
| | | GAGAGCACUGACCCCGAUGGCGAUGGACUACU | |
| | | GAAGCCCUAACUCAGCCAACCUUACUUACAGC | |
| | | AUAAGGGAGCGUAGAAUCUGUGUAGACGAAG | |
| | | GGGGCAUCUGGCCUUACACCUCGUUAGGGAAG | |
| | | AGAAACAGGGUGUUGUCAGCAUCUUCUCACUC | |
| | | CCUUCUCCUUGAUAACAGCUACCAUGACAACC | |
| | | CUGUGGUUUCCAAGGAGCUGAGAAUAGAAGG | |
| | | AAACUAGCUUACAUGAGAACAGACUGGCCUGA | |
| | | GGAGCAGCAGUUGCUGGUGGCUAAUGGUGUA | |
| | | ACCUGAGAUGGCCCUCUGGUAGACACAGGAUA | |
| | | GAUAACUCUUUGGAUAGCAUGUCUUUUUUUC | |
| | | UGUUAAUUAGUUGUGUACUCUGGCCUCUGUC | |
| | | AUAUCUUCACAAUGGUGCUCAUUUCAUGGGG | |
| | | UAUUAUCCAUUCAGUCAUCGUAGGUGAUUUG | |
| | | AAGGUCUUGAUUUGUUUUAGAAUGAUGCACA | |
| | | UUUCAUGUAUUCCAGUUUGUUUAUUACUUAU | |
| | | UUGGGGUUGCAUCAGAAAUGUCUGGAGAAUA | |
| | | AUUCUUUGAUUAUGACUGUUUUUUAAACUAG | |
| | | GAAAAUUGGACAUUAAGCAUCACAAAUGAUA | |
| | | UUAAAAAUUGGCUAGUUGAAUCUAUUGGGAU | |
| | | UUUCUACAAGUAUUCUGCCUUUGCAGAAACAG | |
| | | AUUUGGUGAAUUUGAAUCUCAAUUUGAGUAA | |
| | | UCUGAUCGUUCUUUCUAGCUAAUGGAAAAUG | |
| | | AUUUUACUUAGCAAUGUUAUCUUGGUGUGUU | |
| | | AAGAGUUAGGUUUAACAUAAAGGUUAUUUUC | |
| | | UCCUGAUAUAGAUCACAUAACAGAAUGCACCA | |
| | | GUCAUCAGCUAUUCAGUUGGUAAGCUUCCAGG | |
| | | AAAAAGGACAGGCAGAAAGAGUUUGAGACCU | |
| | | GAAUAGCUCCCAGAUUUCAGUCUUUUCCUGUU | |
| | | UUUGUUAACUUUGGGUUAAAAAAAAAAAAAG | |
| | | UCUGAUUGGUUUUAAUUGAAGGAAAGAUUUG | |
| | | UACUACAGUUCUUUUGUUGUAAAGAGUUGUG | |
| | | UUGUUCUUUUCCCCCAAAGUGGUUUCAGCAAU | |
| | | AUUUAAGGAGAUGUAAGAGCUUUACAAAAAG | |
| | | ACACUUGAUACUUGUUUUCAAACCAGUAUACA | |
| | | AGAUAAGCUUCCAGGCUGCAUAGAAGGAGGA | |
| | | GAGGGAAAAUGUUUUGUAAGAAACCAAUCAA | |
| | | GAUAAAGGACAGUGAAGUAAUCCGUACCUUG | |
| | | UGUUUUGUUUUGAUUUAAUAACAUAACAAAU | |
| | | AACCAACCCUUCCCUGAAAACCUCACAUGCAU | |
| | | ACAUACACAUAUAUACACACACAAAGAGAGUU | |
| | | AAUCAACUGAAAGUGUUUCCUUCAUUUCUGA | |
| | | UAUAGAAUUGCAAUUUUAACACACAUAAAGG | |
| | | AUAAACUUUUAGAAACUUAUCUUACAAAGUG | |
| | | UAUUUUAUAAAAUUAAAGAAAAUAAAAUUAA | |
| | | GAAUGUUCUCAAUCAAAAAAAAAAAAAAA | |
| Mouse | Mouse Plp1 | CUUUUCAUUGCAGGAGAAGAGGACAAAGAUA | 2 |
| (Mm) | mRNA | CUCAGAGAGAAAAAGUAAAGGACAGAAGAAG | |
| | NM_011123.4 | GAGACUGGAGAGACCAGGAUCCUUCCAGCUGA | |
| | (GenBank | GCAAAGUCAGCCGCAAAACAGACUAGCCAACA | |
| | Ref#) | GGCUACAAUUGGAGUCAGAGUGCCAAAGACA | |
| | | UGGGCUUGUUAGAGUGUUGUGCUAGAUGUCU | |
| | | GGUAGGGGCCCCCUUUGCUUCCCUGGUGGCCA | |
| | | CUGGAUUGUGUUUCUUUGGAGUGGCACUGUU | |
| | | CUGUGGAUGUGGACAUGAAGCUCUCACUGGU | |
| | | ACAGAAAAGCUAAUUGAGACCUAUUUCUCCAA | |
| | | AAACUACCAGGACUAUGAGUAUCUCAUUAAU | |
| | | GUGAUUCAUGCUUUCCAGUAUGUCAUCUAUG | |
| | | GAACUGCCUCUUUCUUCUUCCUUUAUGGGGCC | |
| | | CUCCUGCUGGCUGAGGGCUUCUACACCACCGG | |
| | | CGCUGUCAGGCAGAUCUUUGGCGACUACAAGA | |
| | | CCACCAUCUGCGGCAAGGGCCUGAGCGCAACG | |
| | | GUAACAGGGGGCCAGAAGGGGAGGGGUUCCA | |
| | | GAGGCCAACAUCAAGCUCAUUCUUUGGAGCGG | |
| | | GUGUGUCAUUGUUUGGGAAAAUGGCUAGGAC | |
| | | AUCCCGACAAGUUUGUGGGCAUCACCUAUGCC | |
| | | CUGACUGUUGUAUGGCUCCUGGUGUUUGCCUG | |
| | | CUCGGCUGUACCUGUGUACAUUUACUUCAAUA | |
| | | CCUGGACCACCUGUCAGUCUAUUGCCUUCCCU | |
| | | AGCAAGACCUCUGCCAGUAUAGGCAGUCUCUG | |
| | | CGCUGAUGCCAGAAUGUAUGGUGUUCUCCCAU | |
| | | GGAAUGCUUUCCCUGGCAAGGUUUGUGGCUCC | |
| | | AACCUUCUGUCCAUCUGCAAAACAGCUGAGUU | |
| | | CCAAAUGACCUUCCACCUGUUUAUUGCUGCGU | |
| | | UUGUGGGUGCUGCGGCCACACUAGUUUCCCUG | |
| | | CUCACCUUCAUGAUUGCUGCCACUUACAACUU | |
| | | CGCCGUCCUUAAACUCAUGGGCCGAGGCACCA | |
| | | AGUUCUGAGCUCCCAUAGAAACUCCCCUUUGU | |
| | | CUAAUAGCAAGGCUCUAACCACACAGCCUACA | |
| | | GUGUUGUGUUUUAACUCUGCCUUUGCCACUGA | |
| | | UUGGCCCUCUUCUUACUUGAUGAGUAUAACAA | |
| | | GAAAGGAGAGUCUUGCAGUGAUUAAUCUCUC | |
| | | UCUGUGGACUCUCCCUCUUAGUACCUCUUUUA | |
| | | GUCAUUUUGCUCCACAGCAGGCUCCUGCUAGA | |
| | | AAUGGGGGAUGCCUGAGAAGGUGACUCCCCAG | |
| | | CUGCAAGUCGCAGAGGAGUGAAAGCUCUAAU | |
| | | UGAUUUUGCAAGCAUCUCCUGAAGACCAGGAU | |
| | | GUGCUUCCUUCUCAAAGGGCACUUCCAACUGA | |
| | | GGAGAGCAGAACGGAAAGGUUCUCAGGUAGA | |
| | | GAGCAGAAAUGUCCCUGGUCUUCUUGCCAUCA | |
| | | GUAGGAGUCAAAUACAUUCUCUUUGAUGCAC | |
| | | AAAACCAAGAACUCACUCUUACCUUCCUGUUU | |
| | | CCACUGAAGACAGAAGAAAAUAAAAAGAAUG | |
| | | CUAGCAGAGCAAUAUAGCAUUUGCCCAAAUCU | |
| | | GCCUCCUGCAGCUGGGAGAAGGGUGUCAAAGC | |
| | | AAGGAUCUUUCGCCCUUAGAAAGAGAGCUCUG | |
| | | ACGCCAGUGGCAAUGGACUAUUUAAGCCCUAA | |
| | | CUCAGCCAACCUUCCUUACGGCAAUUAGGGAG | |
| | | CACAGUGCCUGUAUAGACAAAGCGGGGCGGAG | |
| | | GGGGGGGGCAUCAUCUGUCCUUAUAGCUCAUU | |
| | | AGGAAGAGAAACAGUGUUGUCAGGAUCAUCU | |
| | | CACUCCCUUCUCCUUGAUAACAGCUACCAUGA | |
| | | CAACCUUGUGGUUUCCAAGGAGCUGAGAAUA | |
| | | GAAAGGAACUAGCUUAUUUGAAAUAAGACUG | |
| | | UGACCUAAGGAGCAUCAGUUGGUGGAUGCUA | |
| | | AAGGUGUAAUUUGAAAUGGCCUUCGGGUAAA | |
| | | UGCAAGAUACUUAACUCUUUGGAUAGCAUGU | |
| | | GUUCUUCCCCCACCCCUAUCCGCUAGUUCUGG | |
| | | CCCCUGGCCUCUGGCAUAAUAUCUUCACAAUG | |
| | | GUGCUUUUUUUCCUGGGGUUUUAUCCAUUCAC | |
| | | UCAUAGCAGGUGAUUAGACGAUCUUGAUUAG | |
| | | UUUCAUAUUUCCCAAUUGUUUAUCUCUUGUU | |
| | | UGGAGUUGUAUCAGAAAGACCUGGAGGAUGA | |
| | | UUCUUUGAGCAUAGUUCUUUUUGAAAACAAG | |
| | | AAAGAGAAACUGGGCAGAAAGCAUCACAAAA | |
| | | AUAUUUGAAAUUGUACGGUCCCAUGAAAUUA | |
| | | UUGGGAAUUCCCCCAAGUAGUCUACCAUUUGU | |
| | | AGAACUAGGCUUGAUAAAUUUGAACCUCAAU | |
| | | UUGAAUAAUUGGUCUGGUAUUUUCUUUUCUA | |
| | | AUAAAUGACAGAUGAUUUUACUUGCUAAUAU | |
| | | UAUCUCAGCAUUUUGAUAAUUUAGGCUUACC | |
| | | AUAGAAGUUACUGUCUCUUGGUAUAUAUAGG | |
| | | UCACAUAAUAGAUUCUGCCAGCUGUUAGCUGU | |
| | | UCAGUUCAUAAGCUUCCAUAGAGCUCUGGAGC | |
| | | CGCAGAGAGGACAGGCAGAAUUUGAAACCUA | |
| | | AAGAACUCCCAGAUUUCAGGCUUAUCCUGUAU | |
| | | UUGUUAACUUUGGGUGAAAGAAAGAAAGAAA | |
| | | GAAAGAAAGAAAGAAAGAAAGAAAGAAAGAA | |
| | | AGAAAGAAAGAAAGAAAGAAAGAAAGAAAGA | |
| | | AAGAAAGAAAGAAAAAGAAAGGAAGGAAGGA | |
| | | AGGAAGGAAGAAAGAAAGAAAGAAAGAAAGA | |
| | | AAGAAAGAAAGAAAGAAAGAAAGAAAGAAAG | |
| | | AAAGAAAGAGAAAAAAAAAGCCCCUGAUCGA | |
| | | AUUUCCUGGAGGAAAAGUUAUUGUAGCUGUU | |
| | | UCAUUGUAGAUUUGUGCUGUCAUUCCCCAAAG | |
| | | UGCUUUCUGCUGUGUUGAAAGAGAUAUAAGA | |
| | | AUUUACAAGAAGACACUUGAGACUUGUUCUU | |
| | | GGGCCAAUAUAUAAGGUAAACAAGCAGGAUG | |
| | | CACAAGAGUGAGGAGAGCUAAAAGGACAUGU | |
| | | AAGAAACCAAUCAAGAUCAAGGAAGGUGAAA | |
| | | UAAUCUAUAUCUUUUAUUUUGUUUUGGUUUA | |
| | | AUAUAACAGAUAACCAACCAUUCCCUUAAAAA | |
| | | UCUCACAUGCACACACACACACACACACACAC | |
| | | ACACGUACAAAGAGAGUUAAUCAACUGCAAG | |
| | | UGUUUCCUUCAUUUCUGAUAGAGAAUUUUGA | |
| | | UUUUAACAACAUAAAGGAUAAACUUUUAGAA | |
| | | ACUCAUCUUACAAAAUGUAUUUUAUAAAAUU | |
| | | AAAGAAAAUAAAAUUAAGAAUGUUCUCAAUC | |
| | | AAACAUCGUGUCCUUUGAGUGAAUUGUUCUA | |
| | | UUUGACCUCAAUAACAGGUACUUAAUUAUAG | |
| | | UUAGCUCGAGGUGCUCAUGUAUCUUUCAGGCC | |
| | | AUGUAAGUUAUUCUUAUACUACUUCUAUGAA | |
| | | AAAUGUAAUAGAUAAUGCAUUAUUAUUAUUA | |
| | | UUGUUUCUUUUUUAUACUAAAGAUAUGAAAA | |
| | | AAUAUAUGCAAAAUGCAAAACAAUUACCGAA | |
| | | AGAAACUCAGUAAAUACUUGUCUCAAAUUGA | |
| Cynomolgus | Monkey | AGAGAGAAAAAGUAAAAGACCGAAGAAGGAG | 3 |
| monkey | PLP1 | GCUGGAGAGACCAGGAUCCUUCUUCCAGCUGA | |
| (Mf) | mRNA | ACAAAGUCAGCCACAAAGCAGACUAGCCAGCC | |
| | NM_001283166.1 | GGCUACAAUUGGAGUCAGAGUCCCAAAGACAU | |
| | (GenBank | GGGCUUGUUAGAGUGCUGUGCAAGAUGUCUG | |
| | Ref#) | GUAGGGGCCCCCUUUGCUUCCCUGGUGGCCAC | |
| | | UGGAUUGUGUUUCUUUGGGGUGGCACUAUUC | |
| | | UGUGGCUGUGGACAUGAAGCCCUCACUGGCAC | |
| | | AGAAAAGCUAAUUGAGACCUAUUUCUCCAAA | |
| | | AACUACCAGGACUAUGAGUAUCUCAUCAAUGU | |
| | | GAUCCAUGCCUUCCAGUAUGUCAUCUAUGGAA | |
| | | CUGCCUCUUUCUUCUUCCUUUAUGGGGCCCUC | |
| | | CUGCUGGCUGAGGGCUUCUACACCACCGGCGC | |
| | | AGUCAGGCAGAUCUUUGGCGACUACAAGACCA | |
| | | CCAUCUGCGGCAAGGGCCUGAGCGCAACGGUA | |
| | | ACAGGGGGCCAGAAGGGGAGGGGUUCCAGAG | |
| | | GCCAACAUCAAGCUCAUUCUUUGGAGCGGGUG | |
| | | UGUCAUUGUUUGGGAAAAUGGCUAGGACAUC | |
| | | CCGACAAGUUUGUGGGCAUCACCUAUGCCCUG | |
| | | ACCGUUGUGUGGCUCCUGGUGUUUGCCUGCUC | |
| | | UGCUGUGCCUGUGUACAUUUACUUCAACACCU | |
| | | GGACCACCUGCCAGUCUAUUGCCUUCCCCAGC | |
| | | AAGACCUCUGCCAGUAUAGGCAGUCUCUGUGC | |
| | | UGAUGCCAGAAUGUAUGGUGUUCUCCCAUGG | |
| | | AAUGCUUUCCCUGGCAAGGUUUGUGGCUCCAA | |
| | | CCUUCUGUCCAUCUGCAAAACAGCUGAGUUCC | |
| | | AAAUGACCUUCCACCUGUUUAUUGCUGCAUUU | |
| | | GUGGGGGCUGCAGCUACACUGAUUUCCCUGCU | |
| | | CACCUUCAUGAUUGCUGCCACUUACAACUUUG | |
| | | CCGUCCUUAAACUCAUGGGCCGAGGCACCAAG | |
| | | UUCUGAUCCCCCAUAGAAAUCCCCCUUUCUCU | |
| | | AAUAGCGAGGCUCUAACCACACAGCCUACAAU | |
| | | GCUGCGUCUCCCAUCUUAACUCUUUGCCUUUG | |
| | | CCACCGACUGGCCCUCUUCUUACUUGACGAGU | |
| | | GUAACAAGAAAGGAGAGUCUUGCAGUGAUUA | |
| | | AGGUCUCUCUUUGGACUCUCCCCUGUUAUGUA | |
| | | CCUCUUUUAGUCAUUUUGCUUCACAGCUGGUU | |
| | | CCUGCUAGAAAUGGGAAAUGCCUAAGAAGAU | |
| | | GACUCCCCAACUGCAAGUCACAAAGGAAUGGA | |
| | | GGCUCUAAUUGAAUUUUCAAGCAUCUCCUGAG | |
| | | GAUCAGAAAGUAAUUUCUUCUCAAAGAGUAC | |
| | | UUCCACUGAUGGAAACAAAGUGGAAGGAAAG | |
| | | AUGCUCAGGUACAGAGAAGGAAUGUCUUUGG | |
| | | UCCCCUUGCCAUUUAUAGGGGCCAAAUAUAUU | |
| | | CUCUUUGGUGUACAA | |
| PLP1- | Unmodified | AUGAGUAUCUCAUUAAUGUAGCAGCCGAAAG | 4 |
| 353 | Sense strand | GCUGC | |
| PLP1- | Unmodified | UACAUUAAUGAGAUACUCAUGG | 5 |
| 353 | antisense | | |
| | strand | | |
| PLP1- | Unmodified | AGUAUCUCAUUAAUGUGAUAGCAGCCGAAAG | 6 |
| 356 | Sense strand | GCUGC | |
| PLP1- | Unmodified | UAUCACAUUAAUGAGAUACUGG | 7 |
| 356 | antisense | | |
| | strand | | |
| PLP1- | Unmodified | AUUAAUGUGAUUCAUGCUUAGCAGCCGAAAG | 8 |
| 364 | Sense strand | GCUGC | |
| PLP1- | Unmodified | UAAGCAUGAAUCACAUUAAUGG | 9 |
| 364 | antisense | | |
| | strand | | |
| PLP1- | Unmodified | AGAAAGCAUCACAAAAAUAAGCAGCCGAAAG | 10 |
| 2191 | Sense strand | GCUGC | |
| PLP1- | Unmodified | UUAUUUUUGUGAUGCUUUCUGG | 11 |
| 2191 | antisense | | |
| | strand | | |
| PLP1- | Unmodified | GAAAGCAUCACAAAAAUAUAGCAGCCGAAAG | 12 |
| 2192 | Sense strand | GCUGC | |
| PLP1- | Unmodified | UAUAUUUUUGUGAUGCUUUCGG | 13 |
| 2192 | antisense | | |
| | strand | | |
| PLP1- | Unmodified | CAUCACAAAAAUAUUUGAAAGCAGCCGAAAG | 14 |
| 2197 | Sense strand | GCUGC | |
| PLP1- | Unmodified | UUUCAAAUAUUUUUGUGAUGGG | 15 |
| 2197 | antisense | | |
| | strand | | |
| PLP1- | Unmodified | ACAGAUGAUUUUACUUGCUAGCAGCCGAAAG | 16 |
| 2339 | Sense strand | GCUGC | |
| PLP1- | Unmodified | UAGCAAGUAAAAUCAUCUGUGG | 17 |
| 2339 | antisense | | |
| | strand | | |
| PLP1- | Unmodified | CAGAUGAUUUUACUUGCUAAGCAGCCGAAAG | 18 |
| 2340 | Sense strand | GCUGC | |
| PLP1- | Unmodified | UUAGCAAGUAAAAUCAUCUGGG | 19 |
| 2340 | antisense | | |
| | strand | | |
| PLP1- | Unmodified | AUUUUACUUGCUAAUAUUAAGCAGCCGAAAG | 20 |
| 2346 | Sense strand | GCUGC | |
| PLP1- | Unmodified | UUAAUAUUAGCAAGUAAAAUGG | 21 |
| 2346 | antisense | | |
| | strand | | |
| PLP1- | Unmodified | AAGUUACUGUCUCUUGGUAAGCAGCCGAAAG | 22 |
| 2398 | Sense strand | GCUGC | |
| PLP1- | Unmodified | UUACCAAGAGACAGUAACUUGG | 23 |
| 2398 | antisense | | |
| | strand | | |
| PLP1- | Unmodified | GGAAAAGUUAUUGUAGCUGAGCAGCCGAAAG | 24 |
| 2779 | Sense strand | GCUGC | |
| PLP1- | Unmodified | UCAGCUACAAUAACUUUUCCGG | 25 |
| 2779 | antisense | | |
| | strand | | |
| PLP1- | Unmodified | GAAAAGUUAUUGUAGCUGUAGCAGCCGAAAG | 26 |
| 2780 | Sense strand | GCUGC | |
| PLP1- | Unmodified | UACAGCUACAAUAACUUUUCGG | 27 |
| 2780 | antisense | | |
| | strand | | |
| PLP1- | Unmodified | GAAGGUGAAAUAAUCUAUAAGCAGCCGAAAG | 28 |
| 2977 | Sense strand | GCUGC | |
| PLP1- | Unmodified | UUAUAGAUUAUUUCACCUUCGG | 29 |
| 2977 | antisense | | |
| | strand | | |
| PLP1- | Unmodified | GUUUUGGUUUAAUAUAACAAGCAGCCGAAAG | 30 |
| 3007 | Sense strand | GCUGC | |
| PLP1- | Unmodified | UUGUUAUAUUAAACCAAAACGG | 31 |
| 3007 | antisense | | |
| | strand | | |
| PLP1- | Unmodified | AUAGAGAAUUUUGAUUUUAAGCAGCCGAAAG | 32 |
| 3130 | Sense strand | GCUGC | |
| PLP1- | Unmodified | UUAAAAUCAAAAUUCUCUAUGG | 33 |
| 3130 | antisense | | |
| | strand | | |
| PLP1- | Unmodified | AGAAUUUUGAUUUUAACAAAGCAGCCGAAAG | 34 |
| 3134 | Sense strand | GCUGC | |
| PLP1- | Unmodified | UUUGUUAAAAUCAAAAUUCUGG | 35 |
| 3134 | antisense | | |
| | strand | | |
| PLP1- | Unmodified | AGUGAAUUGUUCUAUUUGAAGCAGCCGAAAG | 36 |
| 3254 | Sense strand | GCUGC | |
| PLP1- | Unmodified | UUCAAAUAGAACAAUUCACUGG | 37 |
| 3254 | antisense | | |
| | strand | | |
| PLP1- | Unmodified | GUGAAUUGUUCUAUUUGACAGCAGCCGAAAG | 38 |
| 3255 | Sense strand | GCUGC | |
| PLP1- | Unmodified | UGUCAAAUAGAACAAUUCACGG | 39 |
| 3255 | antisense | | |
| | strand | | |
| PLP1- | Modified | [mAs][mU][fG][mA][fG][mU][mA][fU][mC][fU][mC] | 40 |
| 353 | Sense | [fA][fU][mU][fA][mA][fU][mG][mU][mA][mGs][mCs] | |
| | Strand | [mAs][mGs][mCs][mCs][mGs][mAs][mAs][mAs][mGs] | |
| PLP1- | Modified | [MePhosphonate-40- | 41 |
| 353 | antisense | mUs][fAs][fC][fA][fU][mU][fA][mA][mU][fG][mA] | |
| | strand | [mG][mA][fU][mA]fC][mU][mC][fA][mUs][mGs][mG] | |
| PLP1- | Modified | [mAs][mG][fU][mA][fU][mC][mU][fC][mA][fU][mU] | 42 |
| 356 | Sense | [fA][fA][mU][fG][mU][fG][mA][mU][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 43 |
| 356 | antisense | mUs][fAs][fU][fC][fA][mC][fA][mU][mU][fA][mA] | |
| | strand | [mU][mG][fA][mG][fA][mU][mA][fC][mUs][mGs][mG] | |
| PLP1- | Modified | [mAs][mU][fU][mA][fA][mU][mG][fU][mG][fA][mU] | 44 |
| 364 | Sense | [fU][fC][mA][fU][mG][fC][mU][mU][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 45 |
| 364 | antisense | mUs][fAs][fA][fG][fC][mA][fU][mG][mA][fA][mU] | |
| | strand | [mC][mA][fC][mA][fU][mU][mA]fA][mUs][mGs][mG] | |
| PLP1- | Modified | [mAs][mG][fA][mA][fA][mG][mC][fA][mU][fC][mA] | 46 |
| 2191 | Sense | [fC][fA][mA][fA][mA][fA][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 47 |
| 2191 | antisense | mUs][fUs][fA][fU][fU][mU][fU][mU][mG][fU][mG] | |
| | strand | [mA][mU][fG][mC][fU][mU][mU][fC][mUs][mGs][mG] | |
| PLP1- | Modified | [mGs][mA][fA][mA][fG][mC][mA][fU][mC][fA][mC] | 48 |
| 2192 | Sense | [fA][fA][mA][fA][mA][fU][mA][mU][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 49 |
| 2192 | antisense | mUs][fAs][fU][fA][fU][mU][fU][mU][mU][fG][mU] | |
| | strand | [mG][mA][fU][mG][fC][mU][mU][fU][mCs][mGs][mG] | |
| PLP1- | Modified | [mCs][mA][fU][mC][fA][mC][mA][fA][mA][fA][mA] | 50 |
| 2197 | Sense | [fU][fA][mU][fU][mU][fG][mA][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 51 |
| 2197 | antisense | mUs][fUs][fU][fC][fA][mA][fA][mU][mA][fU][mU] | |
| | strand | [mU][mU][fU][mG][fU][mG][mA][fU][mGs][mGs][mG] | |
| PLP1- | Modified | [mAs][mC][fA][mG][fA][mU][mG][fA][mU][fU][mU] | 52 |
| 2339 | Sense | [fU][fA][mC][fU][mU][fG][mC][mU][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 53 |
| 2339 | antisense | mUs][fAs][fG][fC][fA][mA][fG][mU][mA][fA][mA] | |
| | strand | [mA][mU][fC][mA][fU][mC][mU][fG][mUs][mGs][mG] | |
| PLP1- | Modified | [mCs][mA][fG][mA][fU][mG][mA][fU][mU][fU][mU] | 54 |
| 2340 | Sense | [fA][fC][mU][fU][mG][fC][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 55 |
| 2340 | antisense | mUs][fUs][fA][fG][fC][mA][fA][mG][mU][fA][mA] | |
| | strand | [mA][mA][fU][mC][fA][mU][mC][fU][mGs][mGs][mG] | |
| PLP1- | Modified | [mAs][mU][fU][mU][fU][mA][mC][fU][mU][fG][mC] | 56 |
| 2346 | Sense | [fU][fA][mA][fU][mA][fU][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 57 |
| 2346 | antisense | mUs][fUs][fA][fA][fU][mA][fU][mU][mA][fG][mC] | |
| | strand | [mA][mA][fG][mU][fA][mA][mA][fA][mUs][mGs][mG] | |
| PLP1- | Modified | [mAs][mA][fG][mU][fU][mA][mC][fU][mG][fU][mC] | 58 |
| 2398 | Sense | [fU][fC][mU][fU][mG][fG][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 59 |
| 2398 | antisense | mUs][fUs][fA][fC][fC][mA][fA][mG][mA][fG][mA] | |
| | strand | [mC][mA][fG][mU][fA][mA][mC][fU][mUs][mGs][mG] | |
| PLP1- | Modified | [mGs][mG][fA][mA][fA][mA][mG][fU][mU][fA][mU] | 60 |
| 2779 | Sense | [fU][fG][mU][fA][mG][fC][mU][mG][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 61 |
| 2779 | antisense | mUs][fCs][fA][fG][fC][mU][fA][mC][mA][fA][mU] | |
| | strand | [mA][mA][fC][mU][fU][mU][mU][fC][mCs][mGs][mG] | |
| PLP1- | Modified | [mGs][mA][fA][mA][fA][mG][mU][fU][mA][fU][mU] | 62 |
| 2780 | Sense | [fG][fU][mA][fG][mC][fU][mG][mU][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 63 |
| 2780 | antisense | mUs][fAs][fC][fA][fG][mC][fU][mA][mC][fA][mA] | |
| | strand | [mU][mA][fA][mC][fU][mU][mU][fU][mCs][mGs][mG] | |
| PLP1- | Modified | [mGs][mA][fA][mG][fG][mU][mG][fA][mA][fA][mU] | 64 |
| 2977 | Sense | [fA][fA][mU][fC][mU][fA][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 65 |
| 2977 | antisense | mUs][fUs][fA][fU][fA][mG][fA][mU][mU][fA][mU] | |
| | strand | [mU][mU][fC][mA][mC][mC][mU][fU][mCs][mGs][mG] | |
| PLP1- | Modified | [mGs][mU][fU][mU][fU][mG][mG][fU][mU][fU][mA] | 66 |
| 3007 | Sense | [fA][fU][mA][fU][mA][fA][mC][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 67 |
| 3007 | antisense | mUs][fUs][fG][fU][fU][mA][fU][mA][mU][fU][mA] | |
| | strand | [mA][mA][fC][mC][fA][mA][mA][fA][mCs][mGs][mG] | |
| PLP1- | Modified | [mAs][mU][fA][mG][fA][mG][mA][fA][mU][fU][mU] | 68 |
| 3130 | Sense | [fU][fG][mA][fU][mU][fU][mU][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 69 |
| 3130 | antisense | mUs][fUs][fA][fA][fA][mA][fU][mC][mA][fA][mA] | |
| | strand | [mA][mU][fU][mC][fU][mC][mU][fA][mUs][mGs][mG] | |
| PLP1- | Modified | [mAs][mG][fA][mA][fU][mU][mU][fU][mG][fA][mU] | 70 |
| 3134 | Sense | [fU][fU][mU][fA][mA][fC][mA][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 71 |
| 3134 | antisense | mUs][fUs][fU][fG][fU][mU][fA][mA][mA][fA][mU] | |
| | strand | [mC][mA][fA][mA][fA][mU][mU][fC][mUs][mGs][mG] | |
| PLP1- | Modified | [mAs][mG][fU][mG][fA][mA][mU][fU][mG][fU][mU] | 72 |
| 3254 | Sense | [fC][fU][mA][fU][mU][fU][mG][mA][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 73 |
| 3254 | antisense | mUs][fUs][fC][fA][fA][mA][fU][mA][mG][fA][mA] | |
| | strand | [mC][mA][fA][mU][fU][mC]mA][fC][mUs][mGs][mG] | |
| PLP1- | Modified | [mGs][mU][fG][mA][fA][mU][mU][fG][mU][fU][mC] | 74 |
| 3255 | Sense | [fU][fA][mU][fU][mU][fG][mA][mC][mA][mGs][mCs] | |
| | Strand | [mAs][mGs][mCs][mCs][mGs][mAs][mAs][mAs][mGs] | |
| | | [mGs][mCs][mUs][mGs][mC] | |
| PLP1- | Modified | [MePhosphonate-40- | 75 |
| 3255 | antisense | mUs][fGs][fU][fC][fA][mA][fA][mU][mA][fG][mA] | |
| | strand | [mA][mC][fA][mA][fU][mU][mC][fA][mCs][mGs][mG] | |
| PLP1- | Unmodified | ACAGAAAAGCUAAUUGAGAAGCAGCCGAAAG | 76 |
| 436 | Sense strand | GCUGC | |
| PLP1- | Unmodified | UUCUCAAUUAGCUUUUCUGUGG | 77 |
| 436 | antisense | | |
| | strand | | |
| PLP1- | Unmodified | CAGAAAAGCUAAUUGAGACAGCAGCCGAAAG | 78 |
| 437 | Sense strand | GCUGC | |
| PLP1- | Unmodified | UGUCUCAAUUAGCUUUUCUGGG | 79 |
| 437 | antisense | | |
| | strand | | |
| PLP1- | Unmodified | GCUAAUUGAGACCUAUUUCAGCAGCCGAAAGG | 80 |
| 444 | Sense strand | CUGC | |
| PLP1- | Unmodified | UGAAAUAGGUCUCAAUUAGCGG | 81 |
| 444 | antisense | | |
| | strand | | |
| PLP1- | Unmodified | CUAAUUGAGACCUAUUUCUAGCAGCCGAAAGG | 82 |
| 445 | Sense strand | CUGC | |
| PLP1- | Unmodified | UAGAAAUAGGUCUCAAUUAGGG | 83 |
| 445 | antisense | | |
| | strand | | |
| PLP1- | Unmodified | GACUAUGAGUAUCUCAUCAAGCAGCCGAAAGG | 84 |
| 478 | Sense strand | CUGC | |
| PLP1- | Unmodified | UUGAUGAGAUACUCAUAGUCGG | 85 |
| 478 | antisense | | |
| | strand | | |
| PLP1- | Unmodified | ACUAUGAGUAUCUCAUCAAAGCAGCCGAAAGG | 86 |
| 479 | Sense strand | CUGC | |
| PLP1- | Unmodified | UUUGAUGAGAUACUCAUAGUGG | 87 |
| 479 | antisense | | |
| | strand | | |
| PLP1- | Unmodified | AUGAGUAUCUCAUCAAUGUAGCAGCCGAAAG | 88 |
| 482 | Sense strand | GCUGC | |
| PLP1- | Unmodified | UACAUUGAUGAGAUACUCAUGG | 89 |
| 482 | antisense | | |
| | strand | | |
| PLP1- | Unmodified | GAGUAUCUCAUCAAUGUGAAGCAGCCGAAAG | 90 |
| 484 | Sense strand | GCUGC | |
| PLP1- | Unmodified | UUCACAUUGAUGAGAUACUCGG | 91 |
| 484 | antisense | | |
| | strand | | |
| PLP1- | Unmodified | AGUAUCUCAUCAAUGUGAUAGCAGCCGAAAG | 92 |
| 485 | Sense strand | GCUGC | |
| PLP1- | Unmodified | UAUCACAUUGAUGAGAUACUGG | 93 |
| 485 | antisense | | |
| | strand | | |
| PLP1- | Unmodified | CUGUGCCUGUGUACAUUUAAGCAGCCGAAAGG | 94 |
| 821 | Sense strand | CUGC | |
| PLP1- | Unmodified | UUAAAUGUACACAGGCACAGGG | 95 |
| 821 | antisense | | |
| | strand | | |
| PLP1- | Unmodified | CUGUGUACAUUUACUUCAAAGCAGCCGAAAGG | 96 |
| 827 | Sense strand | CUGC | |
| PLP1- | Unmodified | UUUGAAGUAAAUGUACACAGGG | 97 |
| 827 | antisense | | |
| | strand | | |
| PLP1- | Unmodified | GUGUACAUUUACUUCAACAAGCAGCCGAAAGG | 98 |
| 829 | Sense strand | CUGC | |
| PLP1- | Unmodified | UUGUUGAAGUAAAUGUACACGG | 99 |
| 829 | antisense | | |
| | strand | | |
| PLP1- | Unmodified | CCAGAAUGUAUGGUGUUCUAGCAGCCGAAAG | 100 |
| 920 | Sense strand | GCUGC | |
| PLP1- | Unmodified | UAGAACACCAUACAUUCUGGGG | 101 |
| 920 | antisense | | |
| | strand | | |
| PLP1- | Unmodified | CAGCUGAGUUCCAAAUGACAGCAGCCGAAAGG | 102 |
| 998 | Sense strand | CUGC | |
| PLP1- | Unmodified | UGUCAUUUGGAACUCAGCUGGG | 103 |
| 998 | antisense | | |
| | strand | | |
| PLP1- | Unmodified | AAUGACCUUCCACCUGUUUAGCAGCCGAAAGG | 104 |
| 1011 | Sense strand | CUGC | |
| PLP1- | Unmodified | UAAACAGGUGGAAGGUCAUUGG | 105 |
| 1011 | antisense | | |
| | strand | | |
| PLP1- | Unmodified | GACCUUCCACCUGUUUAUUAGCAGCCGAAAGG | 106 |
| 1014 | Sense strand | CUGC | |
| PLP1- | Unmodified | UAAUAAACAGGUGGAAGGUCGG | 107 |
| 1014 | antisense | | |
| | strand | | |
| PLP1- | Unmodified | GCUCACCUUCAUGAUUGCUAGCAGCCGAAAGG | 108 |
| 1071 | Sense strand | CUGC | |
| PLP1- | Unmodified | UAGCAAUCAUGAAGGUGAGCGG | 109 |
| 1071 | antisense | | |
| | strand | | |
| PLP1- | Unmodified | ACCUUCAUGAUUGCUGCCAAGCAGCCGAAAGG | 110 |
| 1075 | Sense strand | CUGC | |
| PLP1- | Unmodified | UUGGCAGCAAUCAUGAAGGUGG | 111 |
| 1075 | antisense | | |
| | strand | | |
| PLP1- | Modified | [mAs][mC][mA][mG][mA][mA][mA][fA][fG][fC][fU] | 112 |
| 436 | Sense | [mA][mA][mU][mU][mG][mA][mG][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 113 |
| 436 | antisense | mUs][fUs][fC][fU][fC][mA][fA][mU][mU][fA][mG] | |
| | strand | [mC][mU][fU][mU][mU][mC][mU][mG][mUs][mGs][mG] | |
| PLP1- | Modified | [mCs][mA][mG][mA][mA][mA][mA][fG][fC][fU][fA] | 114 |
| 437 | Sense | [mA][mU][mU][mG][mA][mG][mA][mC][mA][mG] | |
| | Strand | [mC][mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 115 |
| 437 | antisense | mUs][fGs][fU][fC][fU][mC][fA][mA][mU][fU][mA] | |
| | strand | [mG][mC][fU][mU][mU][mU][mC][mU][mGs][mGs][mG] | |
| PLP1- | Modified | [mGs][mC][mU][mA][mA][mU][mU][fG][fA][fG][fA] | 116 |
| 444 | Sense | [mC][mC][mU][mA][mU][mU][mU][mC][mA][mG] | |
| | Strand | [mC][mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 117 |
| 444 | antisense | mUs][fGs][fA][fA][fA][mU][fA][mG][mG][fU][mC] | |
| | strand | [mU][mC][fA][mA][mU][mU][mA][mG][mCs][mGs][mG] | |
| PLP1- | Modified | [mCs][mU][mA][mA][mU][mU][mG][fA][fG][fA][fC] | 118 |
| 445 | Sense | [mC][mU][mA][mU][mU][mU][mC][mU][mA][mG] | |
| | Strand | [mC][mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 119 |
| 445 | antisense | mUs][fAs][fG][fA][fA][mA][fU][mA][mG][fG][mU] | |
| | strand | [mC][mU][fC][mA][mA][mU][mU][mA][mGs][mGs][mG] | |
| PLP1- | Modified | [mGs][mA][mC][mU][mA][mU][mG][fA][fG][fU][A] | 120 |
| 478 | Sense | [mU][mC][mU][mC][mA][mU][mC][mA][mA][mG] | |
| | Strand | [mC][mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 121 |
| 478 | antisense | mUs][fUs][fG][fA][fU][mG][fA][mG][mA][fU][mA] | |
| | strand | [mC][mU][fC][mA][mU][mA][mG][mU][mCs][mGs][mG] | |
| PLP1- | Modified | [mAs][mC][mU][mA][mU][mG][mA][fG][fU][fA][fU] | 122 |
| 479 | Sense | [mC][mU][mC][mA][mU][mC][mA][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 123 |
| 479 | antisense | mUs][fUs][fU][fG][fA][mU][fG][mA][mG][fA][mU][mA] | |
| | strand | [mC][fU][mC][mA][mU][mA][mG][mUs][mGs][mG] | |
| PLP1- | Modified | [mAs][mu][mg][mA][mg][mu][mA][fu][fc][fu][fc] | 124 |
| 482 | Sense | [mA][mU][mC][mA][mA][mU][mG][mU][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 125 |
| 482 | antisense | mUs][fAs][fC][fA][fU][mU][fG][mA][mU][fG][mA] | |
| | strand | [mG][mA][fU][mA][mC][mU][mC][mA][mUs][mGs][mG] | |
| PLP1- | Modified | [mGs][mA][mG][mU][mA][mU][mC][fU][fC][fA][fU] | 126 |
| 484 | Sense | [mC][mA][mA][mU][mG][mU][mG][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 127 |
| 484 | antisense | mUs][fUs][fC][fA][fC][mA][fU][mU][mG][fA][mU] | |
| | strand | [mG][mA][fG][mA][mU][mA][mC][mU][mCs][mGs][mG] | |
| PLP1- | Modified | [mAs][mG][mU][mA][mU][mC][mU][fC][fA][fU][fC] | 128 |
| 485 | Sense | [mA][mA][mU][mG][mU][mG][mA][mU][mA][mG] | |
| | Strand | [mC][mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 129 |
| 485 | antisense | mUs][fAs][fU][fC][fA][mC][fA][mU][mU][fG][mA] | |
| | strand | [mU][mG][fA][mG][mA][mU][mA][mC][mUs][mGs][mG] | |
| PLP1- | Modified | [mCs][mU][mG][mU][mG][mC][mC][fU][fG][fU][fG] | 130 |
| 821 | Sense | [mU][mA][mC][mA][mU][mU][mU][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 131 |
| 821 | antisense | mUs][fUs][fA][fA][fA][mU][fG][mU][mA][fC][mA] | |
| | strand | [mC][mA][fG][mG][mC][mA][mC][mA][mGs][mGs][mG] | |
| PLP1- | Modified | [mCs][mU][mG][mU][mG][mU][mA][fC][fA][fU][fU] | 132 |
| 827 | Sense | [mU][mA][mC][mU][mU][mC][mA][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 133 |
| 827 | antisense | mUs][fUs][fU][fG][fA][mA][fG][mU][mA][fA][mA] | |
| | strand | [mU][mG][fU][mA][mC][mA][mC][mA][mGs][mGs][mG] | |
| PLP1- | Modified | [mGs][mU][mG][mU][mA][mC][mA][fU][fU][fU][A] | 134 |
| 829 | Sense | [mC][mU][mU][mC][mA][mA][mC][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 135 |
| 829 | antisense | mUs][fUs][fG][fU][fU][mG][fA][mA][mG][fU][mA] | |
| | strand | [mA][mA][fU][mG][mU][mA][mC][mA][mCs][mGs][mG] | |
| PLP1- | Modified | [mCs][mC][mA][mG][mA][mA][mU][fG][fU][fA][fU] | 136 |
| 920 | Sense | [mG][mG][mU][mG][mU][mU][mC][mU][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 137 |
| 920 | antisense | mUs][fAs][fG][fA][fA][mC][fA][mC][mC][fA][mU] | |
| | strand | [mA][mC][fA][mU][mU][mC][mU][mG][mGs][mGs][mG] | |
| PLP1- | Modified | [mCs][mA][mG][mC][mU][mG][mA][fG][fU][fU][fC] | 138 |
| 998 | Sense | [mC][mA][mA][mA][mU][mG][mA][mC][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 139 |
| 998 | antisense | mUs][fGs][fU][fC][fA][mU][fU][mU][mG][fG][mA] | |
| | strand | [mA][mC][fU][mC][mA][mG][mC][mU][mGs][mGs][mG] | |
| PLP1- | Modified | [mAs][mA][mU][mG][mA][mC][mC][fU][fU][fC][fC] | 140 |
| 1011 | Sense | [mA][mC][mC][mU][mG][mU][mU][mU][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 141 |
| 1011 | antisense | mUs][fAs][fA][fA][fC][mA][fG][mG][mU][fG][mG] | |
| | strand | [mA][mA][fG][mG][mU][mC][mA][mU][mUs][mGs][mG] | |
| PLP1- | Modified | [mGs][mA][mC][mC][mU][mU][mC][fC][fA][fC][fC] | 142 |
| 1014 | Sense | [mU][mG][mU][mU][mU][mA][mU][mU][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 143 |
| 1014 | antisense | mUs][fAs][fA][fU][fA][mA][fA][mC][mA][fG][mG] | |
| | strand | [mU][mG][fG][mA][mA][mG][mG][mU][mCs][mGs][mG] | |
| PLP1- | Modified | [mGs][mC][mU][mC][mA][mC][mC][fU][fU][fC][fA] | 144 |
| 1071 | Sense | mU][mG][mA][mU][mU][mG][mC][mU][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 145 |
| 1071 | antisense | mUs][fAs][fG][fC][fA][mA][fU][mC][mA][fU][mG] | |
| | strand | [mA][mA][fG][mG][mU][mG][mA][mG][mCs][mGs][mG] | |
| PLP1- | Modified | [mAs][mC][mC][mU][mU][mC][mA][fU][fG][fA][fU] | 146 |
| 1075 | Sense | [mU][mG][mC][mU][mG][mC][mC][mA][mA][mG][mC] | |
| | Strand | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [MePhosphonate-40- | 147 |
| 1075 | antisense | mUs][fUs][fG][fG][fC][mA][fG][mC][mA][fA][mU] | |
| | strand | [mC][mA][fU][mG][mA][mA][mG][mG][mUs][mGs][mG] | |
| mRNA1 | Mm | AUGAGUAUCUCAUUAAUGUAAUUCA | 148 |
| mRNA2 | Mm | AGUAUCUCAUUAAUGUGAUACAUGC | 149 |
| mRNA3 | Mm | AUUAAUGUGAUUCAUGCUUACCAGT | 150 |
| mRNA4 | Mm | GAGCAUAGUUCUUUUUGAAAACAAG | 151 |
| mRNA5 | Mm | AGCAUAGUUCUUUUUGAAAACAAGA | 152 |
| mRNA6 | Mm | AGAAAGCAUCACAAAAAUAAUUGAA | 153 |
| mRNA7 | Mm | GAAAGCAUCACAAAAAUAUAUGAAA | 154 |
| mRNA8 | Mm | CAUCACAAAAAUAUUUGAAAUUGTA | 155 |
| mRNA9 | Mm | ACAGAUGAUUUUACUUGCUAAUATT | 156 |
| mRNA10 | Mm | CAGAUGAUUUUACUUGCUAAUAUTA | 157 |
| mRNA11 | Mm | AUUUUACUUGCUAAUAUUAACUCAG | 158 |
| mRNA12 | Mm | AAGUUACUGUCUCUUGGUAAAUATA | 159 |
| mRNA13 | Mm | GGAAAAGUUAUUGUAGCUGAUUCAT | 160 |
| mRNA14 | Mm | GAAAAGUUAUUGUAGCUGUAUCATT | 161 |
| mRNA15 | Mm | AAAGUUAUUGUAGCUGUUUAAUUGT | 162 |
| mRNA16 | Mm | AAGUUAUUGUAGCUGUUUCAUUGTA | 163 |
| mRNA17 | Mm | GAAGGUGAAAUAAUCUAUAACUUTT | 164 |
| mRNA18 | Mm | GUUUUGGUUUAAUAUAACAAAUAAC | 165 |
| mRNA19 | Mm | GAUAGAGAAUUUUGAUUUUAACAAC | 166 |
| mRNA20 | Mm | AUAGAGAAUUUUGAUUUUAACAACA | 167 |
| mRNA21 | Mm | AGAAUUUUGAUUUUAACAAAAUAAA | 168 |
| mRNA22 | Mm | AGUGAAUUGUUCUAUUUGAACUCAA | 169 |
| mRNA23 | Mm | GUGAAUUGUUCUAUUUGACAUCAAT | 170 |
| mRNA24 | Hs-Mf-Mm | ACAGAAAAGCUAAUUGAGACCUAUU | 171 |
| mRNA25 | Hs-Mf-Mm | CAGAAAAGCUAAUUGAGACCUAUUU | 172 |
| mRNA26 | Hs-Mf-Mm | GCUAAUUGAGACCUAUUUCUCCAAA | 173 |
| mRNA27 | Hs-Mf-Mm | CUAAUUGAGACCUAUUUCUCCAAAA | 174 |
| mRNA28 | Hs-Mf | GACUAUGAGUAUCUCAUCAAUGUGA | 175 |
| mRNA29 | Hs-Mf | ACUAUGAGUAUCUCAUCAAUGUGAU | 176 |
| mRNA30 | Hs-Mf | AUGAGUAUCUCAUCAAUGUGAUCCA | 177 |
| mRNA31 | Hs-Mf | GAGUAUCUCAUCAAUGUGAUCCAUG | 178 |
| mRNA32 | Hs-Mf | AGUAUCUCAUCAAUGUGAUCCAUGC | 179 |
| mRNA33 | Hs-Mf | CUGUGCCUGUGUACAUUUACUUCAA | 180 |
| mRNA34 | Hs-Mf-Mm | CUGUGUACAUUUACUUCAACACCUG | 181 |
| mRNA35 | Hs-Mf | GUGUACAUUUACUUCAACACCUGGA | 182 |
| mRNA36 | Hs-Mf-Mm | CCAGAAUGUAUGGUGUUCUCCCAUG | 183 |
| mRNA37 | Hs-Mf-Mm | CAGCUGAGUUCCAAAUGACCUUCCA | 184 |
| mRNA38 | Hs-Mf-Mms | AAUGACCUUCCACCUGUUUAUUGCU | 185 |
| mRNA39 | Hs-Mf-Mm | GACCUUCCACCUGUUUAUUGCUGCA | 186 |
| mRNA40 | Hs-Mf-Mm | GCUCACCUUCAUGAUUGCUGCCACU | 187 |
| mRNA41 | Hs-Mf-Mm | ACCUUCAUGAUUGCUGCCACUUACA | 188 |
| Human | PLP1 amino | MGLLECCARCLVGAPFASLVATGLCFFGVALFCG | 189 |
| (Hs) | acid | CGHEALTGTEKLIETYFSKNYQDYEYLINVIHAFQ | |
| | sequence | YVIYGTASFFFLYGALLLAEGFYTTGAVRQIFGDY | |
| | | KTTICGKGLSATVTGGQKGRGSRGQHQAHSLERV | |
| | | CHCLGKWLGHPDKFVGITYALTVVWLLVFACSA | |
| | | VPVYIYFNTWTTCQSIAFPSKTSASIGSLCADARM | |
| | | YGVLPWNAFPGKVCGSNLLSICKTAEFQMTFHLFI | |
| | | AAFVGAAATLVSLLTFMIAATYNFAVLKLMGRG | |
| | | TKF | |
| Stem- | Stem-loop | GCAGCCGAAAGGCUGC | 190 |
| loop | sequence | | |
| GalXC- | Modified | mA][mA][mU][mU][mG][mA][mG][mA][mA][mG] | 191 |
| PLP1- | sense strand | [mC][mA][mG][mC][mC][mG][mA][mA][mA][mG][mG] | |
| 436 | | [mC][mU][mG][mC] | |
| GalXC- | Modified | [MePhosphonate-40- | 192 |
| PLP1- | antisense | mUs][fUs][fC][fU][fC][mA][fA][mU][mU][fA][mG] | |
| 436 | strand | [mC][mU][fU][mU][mU][mC][mU][mG][mUs][mGs][mG] | |
| PLP1- | Modified | [mAs][mC][mA][mG][mA][mU][mG][fA][fU][fU][fU] | 193 |
| 2339 | sense strand | [mU][mA][mC][mU][mU][mG][mC][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [mCs][mA][mG][mA][mU][mG][mA][fU][fU][fU][fU] | 194 |
| 2340 | sense strand | [mA][mC][mU][mU][mG][mC][mU][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [mAs][mA][mG][mU][mU][mA][mC][fU][fG][fU][fC] | 195 |
| 2398 | sense strand | [mU][mC][mU][mU][mG][mG][mU][mA][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [mAs][mA][mG][mU][mU][mA][mC][fU][fG][fU][fC] | 196 |
| 2398 | sense | [mU][mC][mU][mU][mG][mG][mU][mA][mA][mG] | |
| | strand- | [mC][mA][mG][mC][mC][mG][mA][mA][mA][mG][mG] | |
| | | [mC][mU][mG][mC] | |
| PLP1- | Modified | [mCs][mG][mG][mG][mU][mG][mU][fG][fU][fC][fA] | 197 |
| 0718 | sense strand | [mU][mU][mG][mU][mU][mU][mG][mG][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [mGs][mA][mA][mA][mA][mG][mC][fU][fA][fA][fU] | 198 |
| 0439 | sense strand | [mU][mG][mA][mG][mA][mC][mC][mU][mA][mG][mC] | |
| | | [mA][mG][mC][mC][mG][ademA-GalNAc][ademA- | |
| | | GalNAc][ademA- | |
| | | GalNAc][mG][mG][mC][mU][mG][mC] | |
| PLP1- | Modified | [mCs][mA][mG][mA][mU][mG][mA][fU][fU][fU][fU] | 199 |
| 2340 | sense strand | [mA][mC][mU][mU][mG][mC][mU][mA][mA][mG][mC] | |
| | | [mA][mG][mC][mC][mG][mA][mA][mA][mG][mG] | |
| | | [mC][mU][mG][mC] | |
| PLP1- | Modified | [MePhosphonate-40- | 200 |
| 2339 | antisense | mUs][fAs][fG][fC][fA][mA][fG][mU][mA][fA][mA] | |
| | strand | [mA][mU][fC][mA][mU][mC][mU][mG][mUs][mGs][mG] | |
| PLP1- | Modified | [MePhosphonate-40- | 201 |
| 2340 | antisense | mUs][fUs][fA][fG][fC][mA][fA][mG][mU][fA][mA] | |
| | strand | [mA][mA][fU][mC][mA][mU][mC][mU][mGs][mGs][mG] | |
| PLP1- | Modified | [MePhosphonate-40- | 202 |
| 2398 | antisense | mUs][fUs][fA][fC][fC][mA][fA][mG][mA][fG][mA] | |
| | strand | [mC][mA][fG][mU][mA][mA][mC][mU][mUs][mGs][mG] | |
| PLP1- | Modified | [MePhosphonate-40- | 203 |
| 2398 | antisense | mUs][fUs][fAs][fC][fC][mA][fA][mG][mA][fG][mA] | |
| | strand | [mC][mA][fG][mU][mA][mA][mC][mU][mUs][mGs][mG] | |
| PLP1- | Modified | [MePhosphonate-40- | 204 |
| 0718 | antisense | mUs][fCs][fC][fA][fA][mA][fC][mA][mA][fU][mG] | |
| | strand | [mA][mC][fA][mC][mA][mC][mC][mC][mGs][mGs][mG] | |
| PLP1- | Modified | [MePhosphonate-40- | 205 |
| 0439 | antisense | mUs][fAs][fG][fG][fU][mC][fU][mC][mA][fA][mU] | |
| | strand | [mU][mA][fG][mC][mU][mU][mU][mU][mCs][mGs][mG] | |
| PLP1- | Modified | [MePhosphonate-40- | 206 |
| 2340 | antisense | mUs][fUs][fAs][fG][fC][mA][fA][mG][mU][fA][mA] | |
| | strand | [mA][mA][fU][mC][mA][mU][mC][mU][mGs][mGs][mG] | |
| PLP1- | Modified | [MePhosphonate-40- | 207 |
| 0436 | antisense | mUs][fUs][fCs][fU][fC][mA][fA][mU][mU][fA][mG] | |
| | strand | [mC][mU][fU][mU][mU][mC][mU][mG][mUs][mGs][mG] | |
| PLP1- | 36 mer | CGGGUGUGUCAUUGUUUGGAGCAGCCGAAAG | 208 |
| 0718 | Sense strand | GCUGC | |
| PLP1- | 36 mer sense | GAAAAGCUAAUUGAGACCUAGCAGCCGAAAG | 209 |
| 0439 | strand | GCUGC | |
| PLP1- | 22 mer | UCCAAACAAUGACACACCCGGG | 210 |
| 0718 | antisense | | |
| | strand | | |
| PLP1- | 22 mer | UAGGUCUCAAUUAGCUUUUCGG | 211 |
| 0439 | antisense | | |
| | strand | | |
| PLP1- | 19 mer sense | ACAGAAAAGCUAAUUGAGA | 212 |
| 0436 | strand | | |
| PLP1- | 19 mer sense | CAGAAAAGCUAAUUGAGAC | 213 |
| 0437 | strand | | |
| PLP1- | 19 mer sense | GAAAAGCUAAUUGAGACCU | 214 |
| 0439 | strand | | |
| PLP1- | 19 mer sense | GCUAAUUGAGACCUAUUUC | 215 |
| 0444 | strand | | |
| PLP1- | 19 mer sense | CUAAUUGAGACCUAUUUCU | 216 |
| 0445 | strand | | |
| PLP1- | 19 mer sense | GACUAUGAGUAUCUCAUCA | 217 |
| 0478 | strand | | |
| PLP1- | 19 mer sense | ACUAUGAGUAUCUCAUCAA | 218 |
| 0479 | strand | | |
| PLP1- | 19 mer sense | AUGAGUAUCUCAUCAAUGU | 219 |
| 0482 | strand | | |
| PLP1- | 19 mer sense | GAGUAUCUCAUCAAUGUGA | 220 |
| 0484 | strand | | |
| PLP1- | 19 mer sense | AGUAUCUCAUCAAUGUGAU | 221 |
| 0485 | strand | | |
| PLP1- | 19 mer sense | CGGGUGUGUCAUUGUUUGG | 222 |
| 0718 | strand | | |
| PLP1- | 19 mer sense | CUGUGCCUGUGUACAUUUA | 223 |
| 0821 | strand | | |
| PLP1- | 19 mer sense | CUGUGUACAUUUACUUCAA | 224 |
| 0827 | strand | | |
| PLP1- | 19 mer sense | GUGUACAUUUACUUCAACA | 225 |
| 0829 | strand | | |
| PLP1- | 19 mer sense | CCAGAAUGUAUGGUGUUCU | 226 |
| 0920 | strand | | |
| PLP1- | 19 mer sense | CAGCUGAGUUCCAAAUGAC | 227 |
| 0998 | strand | | |
| PLP1- | 19 mer sense | AAUGACCUUCCACCUGUUU | 228 |
| 1011 | strand | | |
| PLP1- | 19 mer sense | GACCUUCCACCUGUUUAUU | 229 |
| 1014 | strand | | |
| PLP1- | 19 mer sense | GCUCACCUUCAUGAUUGCU | 230 |
| 1071 | strand | | |
| PLP1- | 19 mer sense | ACCUUCAUGAUUGCUGCCA | 231 |
| 1075 | strand | | |
| PLP1- | 19 mer sense | ACAGAUGAUUUUACUUGCU | 232 |
| 2339 | strand | | |
| PLP1- | 19 mer sense | CAGAUGAUUUUACUUGCUA | 233 |
| 2340 | strand | | |
| PLP1- | 19 mer sense | AAGUUACUGUCUCUUGGUA | 234 |
| 2398 | strand | | |
| PLP1- | 19 mer anti- | UCUCAAUUAGCUUUUCUGU | 235 |
| 0436 | sense strand | | |
| PLP1- | 19 mer anti- | GUCUCAAUUAGCUUUUCUG | 236 |
| 0437 | sense strand | | |
| PLP1- | 19 mer anti- | AGGUCUCAAUUAGCUUUUC | 237 |
| 0439 | sense strand | | |
| PLP1- | 19 mer anti- | GAAAUAGGUCUCAAUUAGC | 238 |
| 0444 | sense strand | | |
| PLP1- | 19 mer anti- | AGAAAUAGGUCUCAAUUAG | 239 |
| 0445 | sense strand | | |
| PLP1- | 19 mer anti- | UGAUGAGAUACUCAUAGUC | 240 |
| 0478 | sense strand | | |
| PLP1- | 19 mer anti- | UUGAUGAGAUACUCAUAGU | 241 |
| 0479 | sense strand | | |
| PLP1- | 19 mer anti- | ACAUUGAUGAGAUACUCAU | 242 |
| 0482 | sense strand | | |
| PLP1- | 19 mer anti- | UCACAUUGAUGAGAUACUC | 243 |
| 0484 | sense strand | | |
| PLP1- | 19 mer anti- | AUCACAUUGAUGAGAUACU | 244 |
| 0485 | sense strand | | |
| PLP1- | 19 mer anti- | CCAAACAAUGACACACCCG | 245 |
| 0718 | sense strand | | |
| PLP1- | 19 mer anti- | UAAAUGUACACAGGCACAG | 246 |
| 0821 | sense strand | | |
| PLP1- | 19 mer anti- | UUGAAGUAAAUGUACACAG | 247 |
| 0827 | sense strand | | |
| PLP1- | 19 mer anti- | UGUUGAAGUAAAUGUACAC | 248 |
| 0829 | sense strand | | |
| PLP1- | 19mer anti- | AGAACACCAUACAUUCUGG | 249 |
| 0920 | sense strand | | |
| PLP1- | 19 mer anti- | GUCAUUUGGAACUCAGCUG | 250 |
| 0998 | sense strand | | |
| PLP1- | 19 mer anti- | AAACAGGUGGAAGGUCAUU | 251 |
| 1011 | sense strand | | |
| PLP1- | 19 mer anti- | AAUAAACAGGUGGAAGGUC | 252 |
| 1014 | sense strand | | |
| PLP1- | 19 mer anti- | AGCAAUCAUGAAGGUGAGC | 253 |
| 1071 | sense strand | | |
| PLP1- | 19 mer anti- | UGGCAGCAAUCAUGAAGGU | 254 |
| 1075 | sense strand | | |
| PLP1- | 19 mer anti- | AGCAAGUAAAAUCAUCUGU | 255 |
| 2339 | sense strand | | |
| PLP1- | 19 mer anti- | UAGCAAGUAAAAUCAUCUG | 256 |
| 2340 | sense strand | | |
| PLP1- | 19 mer anti- | UACCAAGAGACAGUAACUU | 257 |
| 2398 | sense strand | | |

1. An RNAi oligonucleotide for reducing PLP1 expression, the oligonucleotide comprising a sense strand and an antisense strand, wherein the sense strand and the antisense strand form a duplex region, wherein the antisense strand comprises a region of complementarity to a PLP1 mRNA target sequence of any one of SEQ ID NOs: 171-188 and 212-231, and wherein the region of complementarity is at least 15 contiguous nucleotides in length.

2. The RNAi oligonucleotide of claim 1, wherein (i) the sense strand is 15 to 50 nucleotides in length or 18 to 36 nucleotides in length; and/or (ii) the antisense strand is 15 to 30 nucleotides in length or 22 nucleotides in length.

3.-4. (canceled)

5. The RNAi oligonucleotide of claim 1, wherein the region of complementarity (i) differs by no more than 3 nucleotides in length to the PLP1 mRNA target sequence, or (ii) is fully complementary to the PLP1 mRNA target sequence.

6. The RNAi oligonucleotide of claim 1, wherein the 3′ end of the sense strand comprises a stem-loop set forth as S1-L-S2, wherein S1 is complementary to S2, and wherein L forms a loop between S1 and S2 of 3-5 nucleotides in length.

7. (canceled)

8. The RNAi oligonucleotide of claim 6, wherein at least one nucleotide of L comprises a 2′-O-methyl modification.

9.-10. (canceled)

11. The RNAi oligonucleotide of claim 1, wherein the antisense strand comprises a 3′ overhang sequence of one or more nucleotides in length.

12. The RNAi oligonucleotide of claim 1, wherein the sense strand and/or antisense strand comprise at least one 2′-modified nucleotide selected from the group consisting of 2′-aminoethyl, 2′-fluoro, 2′-O-methyl, 2′-O-methoxyethyl, and 2′-deoxy-2′-fluoro-β-d-arabinonucleic acid.

13.-15. (canceled)

16. The RNAi oligonucleotide of claim 1, wherein (i) the sense strand comprises 36 nucleotides with positions numbered 1-36 from 5′ to 3′, wherein positions 8-11 comprise a 2′-fluoro modification; and/or (ii) the antisense strand comprises 22 nucleotides with positions numbered 1-22 from 5′ to 3′, and wherein positions 2, 3, 4, 5, 7, 10 and 14 comprise a 2′-fluoro modification.

17. The RNAi oligonucleotide of claim 16, wherein the remaining nucleotides of the oligonucleotide comprise a 2′-O-methyl modification.

18. The RNAi oligonucleotide of claim 1, wherein the oligonucleotide comprises at least one modified internucleotide linkage.

19. The RNAi oligonucleotide of claim 1, wherein the 4′-carbon of the sugar of the 5′-nucleotide of the antisense strand comprises a phosphate analog selected from the group consisting of oxymethylphosphonate, vinylphosphonate, and malonylphosphonate.

20.-21. (canceled)

22. The RNAi oligonucleotide of claim 1, wherein
(i) the sense strand comprises a nucleotide sequence selected from any one of SEQ ID NOs: 76, 78, 80, 82, 84, 86, 88, 90, 92, 94, 96, 98, 100, 102, 104, 106, 108, and 110; and/or
(ii) the antisense strand comprises a nucleotide sequence selected from any one of SEQ ID NOs: 77, 79, 81, 83, 85, 87, 89, 91, 93, 95, 97, 99, 101, 103, 105, 107, 109, and 111; or
(iii) the sense strand and antisense strand comprise nucleotide sequences selected from the group consisting of:
(a) SEQ ID NOs: 76 and 77, respectively;
(b) SEQ ID NOs: 78 and 79, respectively;
(c) SEQ ID NOs: 80 and 81, respectively;
(d) SEQ ID NOs: 82 and 83, respectively;
(e) SEQ ID NOs: 84 and 85, respectively;
(f) SEQ ID NOs: 86 and 87, respectively;
(g) SEQ ID NOs: 88 and 89, respectively;
(h) SEQ ID NOs: 90 and 91, respectively;
(i) SEQ ID NOs: 92 and 93, respectively;
(j) SEQ ID NOs: 94 and 95, respectively;
(k) SEQ ID NOs: 96 and 97, respectively;
(l) SEQ ID NOs: 98 and 99, respectively;
(m) SEQ ID NOs: 100 and 101, respectively;
(n) SEQ ID NOs: 102 and 103, respectively;
(o) SEQ ID NOs: 104 and 105, respectively;
(p) SEQ ID NOs: 106 and 107, respectively;
(q) SEQ ID NOs: 108 and 109, respectively; and
(r) SEQ ID NOs: 110 and 111, respectively.

23. The RNAi oligonucleotide of claim 1, wherein:
(i) the sense strand comprises a nucleotide sequence selected from any one of SEQ ID NOs: 112, 114, 116, 118, 120, 122, 124, 126, 128, 130, 132, 134, 136, 138, 140, 142, 144, 146, and 191; and/or
(ii) the antisense strand comprises a nucleotide sequence selected from any one of SEQ ID NOs: 113, 115, 117, 119, 121, 123, 125, 127, 129, 131, 133, 135, 137, 139, 141, 143, 145, 147, and 192; or
(iii) the sense strand and antisense strand comprise nucleotide sequences selected from the group consisting of:
(a) SEQ ID NOs: 112 and 113, respectively;
(b) SEQ ID NOs: 114 and 115, respectively;
(c) SEQ ID NOs: 116 and 117, respectively;
(d) SEQ ID NOs: 118 and 119, respectively;
(e) SEQ ID NOs: 120 and 121, respectively;
(f) SEQ ID NOs: 122 and 123, respectively;
(g) SEQ ID NOs: 124 and 125, respectively;
(h) SEQ ID NOs: 126 and 127, respectively
(i) SEQ ID NOs: 128 and 129, respectively;
(j) SEQ ID NOs: 130 and 131, respectively;
(k) SEQ ID NOs: 131 and 133, respectively;
(l) SEQ ID NOs: 134 and 135, respectively;
(m) SEQ ID NOs: 136 and 137, respectively;
(n) SEQ ID NOs: 138 and 139, respectively;
(o) SEQ ID NOs: 140 and 141, respectively;
(p) SEQ ID NOs: 142 and 143, respectively;
(q) SEQ ID NOs: 144 and 145, respectively;
(r) SEQ ID NOs: 146 and 147, respectively;
(s) SEQ ID NOs: 191 and 192, respectively; and
(t) SEQ ID Nos: 191 and 207, respectively.

24. A pharmaceutical composition comprising the RNAi oligonucleotide of claim 1, and a pharmaceutically acceptable carrier, delivery agent or excipient.

25. The pharmaceutical composition of claim 24, wherein the composition is formulated for administration to the cerebral spinal fluid (CSF).

26. (canceled)

27. A method for treating a subject having a disease, disorder or condition associated with PLP1 expression, the method comprising administering to the subject a therapeutically effective amount of the RNAi oligonucleotide of claim 1, thereby treating the subject.

28. (canceled)

29. The method of claim 27, wherein the disease, disorder or condition associated with PLP1 expression is Pelizaeus-Merzbacher disease (PMD), spastic paraplegia type 2 (SPG2), or reducing astrogliosis or demyelination in the subject.

30.-32. (canceled)

33. A kit comprising the RNAi oligonucleotide of claim 1, a pharmaceutically acceptable carrier, and a package insert comprising instructions for administration to a subject having a disease, disorder or condition associated with PLP1 expression.

34. A method of determining responsiveness to treatment in a patient that has received or is receiving an RNAi oligonucleotide treatment targeting PLP1, wherein the RNAi oligonucleotide is the RNAi oligonucleotide of claim 1, comprising:
determining a level of GFAP expression in a sample from the patient,
wherein reduction in the level of GFAP expression indicates responsiveness to treatment in the patient.

35. A method of determining responsiveness to treatment in a patient with a disease, disorder or condition associated with PLP1 expression, comprising:
(i) administering an RNAi oligonucleotide treatment targeting PLP1 to the patient, wherein the RNAi oligonucleotide is the RNAi oligonucleotide of claim 1; and
(ii) determining a level of GFAP expression in a sample from the patient,
wherein reduction in the level of GFAP expression indicates responsiveness to treatment in the patient.

36. A method of determining responsiveness to treatment in a patient having astrogliosis, comprising:
(i) administering an RNAi oligonucleotide treatment targeting PLP1 to the patient, wherein the RNAi oligonucleotide is the RNAi oligonucleotide of claim 1; and
(ii) determining a level of GFAP expression in a sample from the patient,
wherein reduction in the level of GFAP expression indicates responsiveness to treatment in the patient.

37.-45. (canceled)


SEQUENCE LISTING (257 sequences)

SEQ ID NO: 1 - RNA, 3153 nt, Homo sapiens
acuuucauggcuucucacgcuugugcugcauaucccacaccaauuagacccaaggaucaguuggaaguuuccaggacaucuucauuuuauuuccacccucaauccacauuuccagaugucucugcagcaaagcgaaauuccaggagaagaggacaaagauacucagagagaaaaaguaaaagaccgaagaaggaggcuggagagaccaggauccuuccagcugaacaaagucagccacaaagcagacuagccagccggcuacaauuggagucagagucccaaagacaugggcuuguuagagugcugugcaagaugucugguaggggcccccuuugcuucccugguggccacuggauuguguuucuuugggguggcacuguucuguggcuguggacaugaagcccucacuggcacagaaaagcuaauugagaccuauuucuccaaaaacuaccaagacuaugaguaucucaucaaugugauccaugccuuccaguaugucaucuauggaacugccucuuucuucuuccuuuauggggcccuccugcuggcugagggcuucuacaccaccggcgcagucaggcagaucuuuggcgacuacaagaccaccaucugcggcaagggccugagcgcaacgguaacagggggccagaaggggagggguuccagaggccaacaucaagcucauucuuuggagcgggugugucauuguuugggaaaauggcuaggacaucccgacaaguuugugggcaucaccuaugcccugaccguuguguggcuccugguguuugccugcucugcugugccuguguacauuuacuucaacaccuggaccaccugccagucuauugccuuccccagcaagaccucugccaguauaggcagucucugugcugaugccagaauguaugguguucucccauggaaugcuuucccuggcaagguuuguggcuccaaccuucuguccaucugcaaaacagcugaguuccaaaugaccuuccaccuguuuauugcugcauuugugggggcugcagcuacacugguuucccugcucaccuucaugauugcugccacuuacaacuuugccguccuuaaacucaugggccgaggcaccaaguucugaucccccguagaaaucccccuuucucuaauagcgaggcucuaaccacacagccuacaaugcugcgucucccaucuuaacucuuugccuuugccaccaacuggcccucuucuuacuugaugaguguaacaagaaaggagagucuugcagugauuaaggucucucuuuggacucuccccucuuauguaccucuuuuagucauuuugcuucauagcugguuccugcuagaaaugggaaaugccuaagaagaugacuucccaacugcaagucacaaaggaauggaggcucuaauugaauuuucaagcaucuccugaggaucagaaaguaauuucuucucaaaggguacuuccacugauggaaacaaaguggaaggaaagaugcucagguacagagaaggaaugucuuugguccucuugccaucuauaggggccaaauauauucucuuugguguacaaaauggaauucauucuggucucucuauuaccacugaagauagaagaaaaaagaaugucagaaaaacaauaagagcguuugcccaaaucugccuauugcagcugggagaagggggucaaagcaaggaucuuucacccacagaaagagagcacugaccccgauggcgauggacuacugaagcccuaacucagccaaccuuacuuacagcauaagggagcguagaaucuguguagacgaagggggcaucuggccuuacaccucguuagggaagagaaacaggguguugucagcaucuucucacucccuucuccuugauaacagcuaccaugacaacccugugguuuccaaggagcugagaauagaaggaaacuagcuuacaugagaacagacuggccugaggagcagcaguugcugguggcuaaugguguaaccugagauggcccucugguagacacaggauagauaacucuuuggauagcaugucuuuuuuucuguuaauuaguuguguacucuggccucugucauaucuucacaauggugcucauuucaugggguauuauccauucagucaucguaggugauuugaaggucuugauuuguuuuagaaugaugcacauuucauguauuccaguuuguuuauuacuuauuugggguugcaucagaaaugucuggagaauaauucuuugauuaugacuguuuuuuaaacuaggaaaauuggacauuaagcaucacaaaugauauuaaaaauuggcuaguugaaucuauugggauuuucuacaaguauucugccuuugcagaaacagauuuggugaauuugaaucucaauuugaguaaucugaucguucuuucuagcuaauggaaaaugauuuuacuuagcaauguuaucuugguguguuaagaguuagguuuaacauaaagguuauuuucuccugauauagaucacauaacagaaugcaccagucaucagcuauucaguugguaagcuuccaggaaaaaggacaggcagaaagaguuugagaccugaauagcucccagauuucagucuuuuccuguuuuuguuaacuuuggguuaaaaaaaaaaaaagucugauugguuuuaauugaaggaaagauuuguacuacaguucuuuuguuguaaagaguuguguuguucuuuucccccaaagugguuucagcaauauuuaaggagauguaagagcuuuacaaaaagacacuugauacuuguuuucaaaccaguauacaagauaagcuuccaggcugcauagaaggaggagagggaaaauguuuuguaagaaaccaaucaagauaaaggacagugaaguaauccguaccuuguguuuuguuuugauuuaauaacauaacaaauaaccaacccuucccugaaaaccucacaugcauacauacacauauauacacacacaaagagaguuaaucaacugaaaguguuuccuucauuucugauauagaauugcaauuuuaacacacauaaaggauaaacuuuuagaaacuuaucuuacaaaguguauuuuauaaaauuaaagaaaauaaaauuaagaauguucucaaucaaaaaaaaaaaaaaa

SEQ ID NO: 2 - RNA, 3461 nt, Mus musculus
cuuuucauugcaggagaagaggacaaagauacucagagagaaaaaguaaaggacagaagaaggagacuggagagaccaggauccuuccagcugagcaaagucagccgcaaaacagacuagccaacaggcuacaauuggagucagagugccaaagacaugggcuuguuagaguguugugcuagaugucugguaggggcccccuuugcuucccugguggccacuggauuguguuucuuuggaguggcacuguucuguggauguggacaugaagcucucacugguacagaaaagcuaauugagaccuauuucuccaaaaacuaccaggacuaugaguaucucauuaaugugauucaugcuuuccaguaugucaucuauggaacugccucuuucuucuuccuuuauggggcccuccugcuggcugagggcuucuacaccaccggcgcugucaggcagaucuuuggcgacuacaagaccaccaucugcggcaagggccugagcgcaacgguaacagggggccagaaggggagggguuccagaggccaacaucaagcucauucuuuggagcgggugugucauuguuugggaaaauggcuaggacaucccgacaaguuugugggcaucaccuaugcccugacuguuguauggcuccugguguuugccugcucggcuguaccuguguacauuuacuucaauaccuggaccaccugucagucuauugccuucccuagcaagaccucugccaguauaggcagucucugcgcugaugccagaauguaugguguucucccauggaaugcuuucccuggcaagguuuguggcuccaaccuucuguccaucugcaaaacagcugaguuccaaaugaccuuccaccuguuuauugcugcguuugugggugcugcggccacacuaguuucccugcucaccuucaugauugcugccacuuacaacuucgccguccuuaaacucaugggccgaggcaccaaguucugagcucccauagaaacuccccuuugucuaauagcaaggcucuaaccacacagccuacaguguuguguuuuaacucugccuuugccacugauuggcccucuucuuacuugaugaguauaacaagaaaggagagucuugcagugauuaaucucucucuguggacucucccucuuaguaccucuuuuagucauuuugcuccacagcaggcuccugcuagaaaugggggaugccugagaaggugacuccccagcugcaagucgcagaggagugaaagcucuaauugauuuugcaagcaucuccugaagaccaggaugugcuuccuucucaaagggcacuuccaacugaggagagcagaacggaaagguucucagguagagagcagaaaugucccuggucuucuugccaucaguaggagucaaauacauucucuuugaugcacaaaaccaagaacucacucuuaccuuccuguuuccacugaagacagaagaaaauaaaaagaaugcuagcagagcaauauagcauuugcccaaaucugccuccugcagcugggagaagggugucaaagcaaggaucuuucgcccuuagaaagagagcucugacgccaguggcaauggacuauuuaagcccuaacucagccaaccuuccuuacggcaauuagggagcacagugccuguauagacaaagcggggcggagggggggggcaucaucuguccuuauagcucauuaggaagagaaacaguguugucaggaucaucucacucccuucuccuugauaacagcuaccaugacaaccuugugguuuccaaggagcugagaauagaaaggaacuagcuuauuugaaauaagacugugaccuaaggagcaucaguugguggaugcuaaagguguaauuugaaauggccuucggguaaaugcaagauacuuaacucuuuggauagcauguguucuucccccaccccuauccgcuaguucuggccccuggccucuggcauaauaucuucacaauggugcuuuuuuuccugggguuuuauccauucacucauagcaggugauuagacgaucuugauuaguuucauauuucccaauuguuuaucucuuguuuggaguuguaucagaaagaccuggaggaugauucuuugagcauaguucuuuuugaaaacaagaaagagaaacugggcagaaagcaucacaaaaauauuugaaauuguacggucccaugaaauuauugggaauucccccaaguagucuaccauuuguagaacuaggcuugauaaauuugaaccucaauuugaauaauuggucugguauuuucuuuucuaauaaaugacagaugauuuuacuugcuaauauuaucucagcauuuugauaauuuaggcuuaccauagaaguuacugucucuugguauauauaggucacauaauagauucugccagcuguuagcuguucaguucauaagcuuccauagagcucuggagccgcagagaggacaggcagaauuugaaaccuaaagaacucccagauuucaggcuuauccuguauuuguuaacuuugggugaaagaaagaaagaaagaaagaaagaaagaaagaaagaaagaaagaaagaaagaaagaaagaaagaaagaaagaaagaaagaaagaaagaaaaagaaaggaaggaaggaaggaaggaagaaagaaagaaagaaagaaagaaagaaagaaagaaagaaagaaagaaagaaagaaagaaagagaaaaaaaaagccccugaucgaauuuccuggaggaaaaguuauuguagcuguuucauuguagauuugugcugucauuccccaaagugcuuucugcuguguugaaagagauauaagaauuuacaagaagacacuugagacuuguucuugggccaauauauaagguaaacaagcaggaugcacaagagugaggagagcuaaaaggacauguaagaaaccaaucaagaucaaggaaggugaaauaaucuauaucuuuuauuuuguuuugguuuaauauaacagauaaccaaccauucccuuaaaaaucucacaugcacacacacacacacacacacacacacguacaaagagaguuaaucaacugcaaguguuuccuucauuucugauagagaauuuugauuuuaacaacauaaaggauaaacuuuuagaaacucaucuuacaaaauguauuuuauaaaauuaaagaaaauaaaauuaagaauguucucaaucaaacaucguguccuuugagugaauuguucuauuugaccucaauaacagguacuuaauuauaguuagcucgaggugcucauguaucuuucaggccauguaaguuauucuuauacuacuucuaugaaaaauguaauagauaaugcauuauuauuauuauuguuucuuuuuuauacuaaagauaugaaaaaauauaugcaaaaugcaaaacaauuaccgaaagaaacucaguaaauacuugucucaaauuga

SEQ ID NO: 3 - RNA, 1411 nt, Macaca fascicularis
agagagaaaaaguaaaagaccgaagaaggaggcuggagagaccaggauccuucuuccagcugaacaaagucagccacaaagcagacuagccagccggcuacaauuggagucagagucccaaagacaugggcuuguuagagugcugugcaagaugucugguaggggcccccuuugcuucccugguggccacuggauuguguuucuuugggguggcacuauucuguggcuguggacaugaagcccucacuggcacagaaaagcuaauugagaccuauuucuccaaaaacuaccaggacuaugaguaucucaucaaugugauccaugccuuccaguaugucaucuauggaacugccucuuucuucuuccuuuauggggcccuccugcuggcugagggcuucuacaccaccggcgcagucaggcagaucuuuggcgacuacaagaccaccaucugcggcaagggccugagcgcaacgguaacagggggccagaaggggagggguuccagaggccaacaucaagcucauucuuuggagcgggugugucauuguuugggaaaauggcuaggacaucccgacaaguuugugggcaucaccuaugcccugaccguuguguggcuccugguguuugccugcucugcugugccuguguacauuuacuucaacaccuggaccaccugccagucuauugccuuccccagcaagaccucugccaguauaggcagucucugugcugaugccagaauguaugguguucucccauggaaugcuuucccuggcaagguuuguggcuccaaccuucuguccaucugcaaaacagcugaguuccaaaugaccuuccaccuguuuauugcugcauuugugggggcugcagcuacacugauuucccugcucaccuucaugauugcugccacuuacaacuuugccguccuuaaacucaugggccgaggcaccaaguucugaucccccauagaaaucccccuuucucuaauagcgaggcucuaaccacacagccuacaaugcugcgucucccaucuuaacucuuugccuuugccaccgacuggcccucuucuuacuugacgaguguaacaagaaaggagagucuugcagugauuaaggucucucuuuggacucuccccuguuauguaccucuuuuagucauuuugcuucacagcugguuccugcuagaaaugggaaaugccuaagaagaugacuccccaacugcaagucacaaaggaauggaggcucuaauugaauuuucaagcaucuccugaggaucagaaaguaauuucuucucaaagaguacuuccacugauggaaacaaaguggaaggaaagaugcucagguacagagaaggaaugucuuugguccccuugccauuuauaggggccaaauauauucucuuugguguacaa

SEQ ID NO: 4 - RNA, 36 nt, Artificial Sequence
  synthetic construct
augaguaucucauuaauguagcagccgaaaggcugc

SEQ ID NO: 5 - RNA, 22 nt, Artificial Sequence
  synthetic construct
uacauuaaugagauacucaugg

SEQ ID NO: 6 - RNA, 36 nt, Artificial Sequence
  synthetic construct
aguaucucauuaaugugauagcagccgaaaggcugc

SEQ ID NO: 7 - RNA, 22 nt, Artificial Sequence
  synthetic construct
uaucacauuaaugagauacugg

SEQ ID NO: 8 - RNA, 36 nt, Artificial Sequence
  synthetic construct
auuaaugugauucaugcuuagcagccgaaaggcugc

SEQ ID NO: 9 - RNA, 22 nt, Artificial Sequence
  synthetic construct
uaagcaugaaucacauuaaugg

SEQ ID NO: 10 - RNA, 36 nt, Artificial Sequence
  synthetic construct
agaaagcaucacaaaaauaagcagccgaaaggcugc

SEQ ID NO: 11 - RNA, 22 nt, Artificial Sequence
  synthetic construct
uuauuuuugugaugcuuucugg

SEQ ID NO: 12 - RNA, 36 nt, Artificial Sequence
  synthetic construct
gaaagcaucacaaaaauauagcagccgaaaggcugc

SEQ ID NO: 13 - RNA, 22 nt, Artificial Sequence
  synthetic construct
uauauuuuugugaugcuuucgg

SEQ ID NO: 14 - RNA, 36 nt, Artificial Sequence
  synthetic construct
caucacaaaaauauuugaaagcagccgaaaggcugc

SEQ ID NO: 15 - RNA, 22 nt, Artificial Sequence
  synthetic construct
uuucaaauauuuuugugauggg

SEQ ID NO: 16 - RNA, 36 nt, Artificial Sequence
  synthetic construct
acagaugauuuuacuugcuagcagccgaaaggcugc

SEQ ID NO: 17 - RNA, 22 nt, Artificial Sequence
  synthetic construct
uagcaaguaaaaucaucugugg

SEQ ID NO: 18 - RNA, 36 nt, Artificial Sequence
  synthetic construct
cagaugauuuuacuugcuaagcagccgaaaggcugc

SEQ ID NO: 19 - RNA, 22 nt, Artificial Sequence
  synthetic construct
uuagcaaguaaaaucaucuggg

SEQ ID NO: 20 - RNA, 36 nt, Artificial Sequence
  synthetic construct
auuuuacuugcuaauauuaagcagccgaaaggcugc

SEQ ID NO: 21 - RNA, 22 nt, Artificial Sequence
  synthetic construct
uuaauauuagcaaguaaaaugg

SEQ ID NO: 22 - RNA, 36 nt, Artificial Sequence
  synthetic construct
aaguuacugucucuugguaagcagccgaaaggcugc

SEQ ID NO: 23 - RNA, 22 nt, Artificial Sequence
  synthetic construct
uuaccaagagacaguaacuugg

SEQ ID NO: 24 - RNA, 36 nt, Artificial Sequence
  synthetic construct
ggaaaaguuauuguagcugagcagccgaaaggcugc

SEQ ID NO: 25 - RNA, 22 nt, Artificial Sequence
  synthetic construct
ucagcuacaauaacuuuuccgg

SEQ ID NO: 26 - RNA, 36 nt, Artificial Sequence
  synthetic construct
gaaaaguuauuguagcuguagcagccgaaaggcugc

SEQ ID NO: 27 - RNA, 22 nt, Artificial Sequence
  synthetic construct
uacagcuacaauaacuuuucgg

SEQ ID NO: 28 - RNA, 36 nt, Artificial Sequence
  synthetic construct
gaaggugaaauaaucuauaagcagccgaaaggcugc

SEQ ID NO: 29 - RNA, 22 nt, Artificial Sequence
  synthetic construct
uuauagauuauuucaccuucgg

SEQ ID NO: 30 - RNA, 36 nt, Artificial Sequence
  synthetic construct
guuuugguuuaauauaacaagcagccgaaaggcugc

SEQ ID NO: 31 - RNA, 22 nt, Artificial Sequence
  synthetic construct
uuguuauauuaaaccaaaacgg

SEQ ID NO: 32 - RNA, 36 nt, Artificial Sequence
  synthetic construct
auagagaauuuugauuuuaagcagccgaaaggcugc

SEQ ID NO: 33 - RNA, 22 nt, Artificial Sequence
  synthetic construct
uuaaaaucaaaauucucuaugg

SEQ ID NO: 34 - RNA, 36 nt, Artificial Sequence
  synthetic construct
agaauuuugauuuuaacaaagcagccgaaaggcugc

SEQ ID NO: 35 - RNA, 22 nt, Artificial Sequence
  synthetic construct
uuuguuaaaaucaaaauucugg

SEQ ID NO: 36 - RNA, 36 nt, Artificial Sequence
  synthetic construct
agugaauuguucuauuugaagcagccgaaaggcugc

SEQ ID NO: 37 - RNA, 22 nt, Artificial Sequence
  synthetic construct
uucaaauagaacaauucacugg

SEQ ID NO: 38 - RNA, 36 nt, Artificial Sequence
  synthetic construct
gugaauuguucuauuugacagcagccgaaaggcugc

SEQ ID NO: 39 - RNA, 22 nt, Artificial Sequence
  synthetic construct
ugucaaauagaacaauucacgg

SEQ ID NO: 40 - RNA, 36 nt, Artificial Sequence; identical to SEQ ID NO: 4
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 41 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uacauuaaugagauacucaugg

SEQ ID NO: 42 - RNA, 36 nt, Artificial Sequence; identical to SEQ ID NO: 6
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 43 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaucacauuaaugagauacugg

SEQ ID NO: 44 - RNA, 36 nt, Artificial Sequence; identical to SEQ ID NO: 8
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 45 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaagcaugaaucacauuaaugg

SEQ ID NO: 46 - RNA, 36 nt, Artificial Sequence; identical to SEQ ID NO: 10
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 47 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuauuuuugugaugcuuucugg

SEQ ID NO: 48 - RNA, 36 nt, Artificial Sequence; identical to SEQ ID NO: 12
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 49 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uauauuuuugugaugcuuucgg

SEQ ID NO: 50 - RNA, 36 nt, Artificial Sequence; identical to SEQ ID NO: 14
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 51 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuucaaauauuuuugugauggg

SEQ ID NO: 52 - RNA, 36 nt, Artificial Sequence; identical to SEQ ID NO: 16
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 53 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uagcaaguaaaaucaucugugg

SEQ ID NO: 54 - RNA, 36 nt, Artificial Sequence; identical to SEQ ID NO: 18
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 55 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuagcaaguaaaaucaucuggg

SEQ ID NO: 56 - RNA, 36 nt, Artificial Sequence; identical to SEQ ID NO: 20
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 57 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaauauuagcaaguaaaaugg

SEQ ID NO: 58 - RNA, 36 nt, Artificial Sequence; identical to SEQ ID NO: 22
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 59 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaccaagagacaguaacuugg

SEQ ID NO: 60 - RNA, 36 nt, Artificial Sequence; identical to SEQ ID NO: 24
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 61 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ucagcuacaauaacuuuuccgg

SEQ ID NO: 62 - RNA, 36 nt, Artificial Sequence; identical to SEQ ID NO: 26
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 63 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uacagcuacaauaacuuuucgg

SEQ ID NO: 64 - RNA, 36 nt, Artificial Sequence; identical to SEQ ID NO: 28
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 65 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuauagauuauuucaccuucgg

SEQ ID NO: 66 - RNA, 36 nt, Artificial Sequence; identical to SEQ ID NO: 30
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 67 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuguuauauuaaaccaaaacgg

SEQ ID NO: 68 - RNA, 36 nt, Artificial Sequence; identical to SEQ ID NO: 32
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 69 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaaaaucaaaauucucuaugg

SEQ ID NO: 70 - RNA, 36 nt, Artificial Sequence; identical to SEQ ID NO: 34
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 71 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuuguuaaaaucaaaauucugg

SEQ ID NO: 72 - RNA, 36 nt, Artificial Sequence; identical to SEQ ID NO: 36
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 73 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uucaaauagaacaauucacugg

SEQ ID NO: 74 - RNA, 36 nt, Artificial Sequence; identical to SEQ ID NO: 38
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-fluoro modified
  modified_base (4)..(4): 2′-O-methyl modified
  modified_base (5)..(5): 2′-fluoro modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-fluoro modified
  modified_base (13)..(13): 2′-fluoro modified
  modified_base (14)..(14): 2′-O-methyl modified
  modified_base (15)..(15): 2′-fluoro modified
  modified_base (16)..(16): 2′-O-methyl modified
  modified_base (17)..(17): 2′-fluoro modified
  modified_base (18)..(18): 2′-O-methyl modified
  modified_base (19)..(19): 2′-O-methyl modified
  modified_base (20)..(20): 2′-O-methyl modified
  misc_feature (21)..(36): linked via phosphorothioate
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 75 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-fluoro modified
  modified_base (17)..(17): 2′-O-methyl modified
  modified_base (18)..(18): 2′-O-methyl modified
  modified_base (19)..(19): 2′-fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ugucaaauagaacaauucacgg

SEQ ID NO: 76 - RNA, 36 nt, Artificial Sequence
  synthetic construct
acagaaaagcuaauugagaagcagccgaaaggcugc

SEQ ID NO: 77 - RNA, 22 nt, Artificial Sequence
  synthetic construct
uucucaauuagcuuuucugugg

SEQ ID NO: 78 - RNA, 36 nt, Artificial Sequence
  synthetic construct
cagaaaagcuaauugagacagcagccgaaaggcugc

SEQ ID NO: 79 - RNA, 22 nt, Artificial Sequence
  synthetic construct
ugucucaauuagcuuuucuggg

SEQ ID NO: 80 - RNA, 36 nt, Artificial Sequence
  synthetic construct
gcuaauugagaccuauuucagcagccgaaaggcugc

SEQ ID NO: 81 - RNA, 22 nt, Artificial Sequence
  synthetic construct
ugaaauaggucucaauuagcgg

SEQ ID NO: 82 - RNA, 36 nt, Artificial Sequence
  synthetic construct
cuaauugagaccuauuucuagcagccgaaaggcugc

SEQ ID NO: 83 - RNA, 22 nt, Artificial Sequence
  synthetic construct
uagaaauaggucucaauuaggg

SEQ ID NO: 84 - RNA, 36 nt, Artificial Sequence
  synthetic construct
gacuaugaguaucucaucaagcagccgaaaggcugc

SEQ ID NO: 85 - RNA, 22 nt, Artificial Sequence
  synthetic construct
uugaugagauacucauagucgg

SEQ ID NO: 86 - RNA, 36 nt, Artificial Sequence
  synthetic construct
acuaugaguaucucaucaaagcagccgaaaggcugc

SEQ ID NO: 87 - RNA, 22 nt, Artificial Sequence
  synthetic construct
uuugaugagauacucauagugg

SEQ ID NO: 88 - RNA, 36 nt, Artificial Sequence
  synthetic construct
augaguaucucaucaauguagcagccgaaaggcugc

SEQ ID NO: 89 - RNA, 22 nt, Artificial Sequence
  synthetic construct
uacauugaugagauacucaugg

SEQ ID NO: 90 - RNA, 36 nt, Artificial Sequence
  synthetic construct
gaguaucucaucaaugugaagcagccgaaaggcugc

SEQ ID NO: 91 - RNA, 22 nt, Artificial Sequence
  synthetic construct
uucacauugaugagauacucgg

SEQ ID NO: 92 - RNA, 36 nt, Artificial Sequence
  synthetic construct
aguaucucaucaaugugauagcagccgaaaggcugc

SEQ ID NO: 93 - RNA, 22 nt, Artificial Sequence
  synthetic construct
uaucacauugaugagauacugg

SEQ ID NO: 94 - RNA, 36 nt, Artificial Sequence
  synthetic construct
cugugccuguguacauuuaagcagccgaaaggcugc

SEQ ID NO: 95 - RNA, 22 nt, Artificial Sequence
  synthetic construct
uuaaauguacacaggcacaggg

SEQ ID NO: 96 - RNA, 36 nt, Artificial Sequence
  synthetic construct
cuguguacauuuacuucaaagcagccgaaaggcugc

SEQ ID NO: 97 - RNA, 22 nt, Artificial Sequence
  synthetic construct
uuugaaguaaauguacacaggg

SEQ ID NO: 98 - RNA, 36 nt, Artificial Sequence
  synthetic construct
guguacauuuacuucaacaagcagccgaaaggcugc

SEQ ID NO: 99 - RNA, 22 nt, Artificial Sequence
  synthetic construct
uuguugaaguaaauguacacgg

SEQ ID NO: 100 - RNA, 36 nt, Artificial Sequence
  synthetic construct
ccagaauguaugguguucuagcagccgaaaggcugc

SEQ ID NO: 101 - RNA, 22 nt, Artificial Sequence
  synthetic construct
uagaacaccauacauucugggg

SEQ ID NO: 102 - RNA, 36 nt, Artificial Sequence
  synthetic construct
cagcugaguuccaaaugacagcagccgaaaggcugc

SEQ ID NO: 103 - RNA, 22 nt, Artificial Sequence
  synthetic construct
ugucauuuggaacucagcuggg

SEQ ID NO: 104 - RNA, 36 nt, Artificial Sequence
  synthetic construct
aaugaccuuccaccuguuuagcagccgaaaggcugc

SEQ ID NO: 105 - RNA, 22 nt, Artificial Sequence
  synthetic construct
uaaacagguggaaggucauugg

SEQ ID NO: 106 - RNA, 36 nt, Artificial Sequence
  synthetic construct
gaccuuccaccuguuuauuagcagccgaaaggcugc

SEQ ID NO: 107 - RNA, 22 nt, Artificial Sequence
  synthetic construct
uaauaaacagguggaaggucgg

SEQ ID NO: 108 - RNA, 36 nt, Artificial Sequence
  synthetic construct
gcucaccuucaugauugcuagcagccgaaaggcugc

SEQ ID NO: 109 - RNA, 22 nt, Artificial Sequence
  synthetic construct
uagcaaucaugaaggugagcgg

SEQ ID NO: 110 - RNA, 36 nt, Artificial Sequence
  synthetic construct
accuucaugauugcugccaagcagccgaaaggcugc

SEQ ID NO: 111 - RNA, 22 nt, Artificial Sequence
  synthetic construct
uuggcagcaaucaugaaggugg

SEQ ID NO: 112 - RNA, 36 nt, Artificial Sequence; identical to SEQ ID NO: 76
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 113 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uucucaauuagcuuuucugugg

SEQ ID NO: 114 - RNA, 36 nt, Artificial Sequence; identical to SEQ ID NO: 78
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 115 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ugucucaauuagcuuuucuggg

SEQ ID NO: 116 - RNA, 36 nt, Artificial Sequence; identical to SEQ ID NO: 80
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 117 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ugaaauaggucucaauuagcgg

SEQ ID NO: 118 - RNA, 36 nt, Artificial Sequence; identical to SEQ ID NO: 82
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 119 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uagaaauaggucucaauuaggg

SEQ ID NO: 120 - RNA, 36 nt, Artificial Sequence; identical to SEQ ID NO: 84
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 121 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uugaugagauacucauagucgg

SEQ ID NO: 122 - RNA, 36 nt, Artificial Sequence; identical to SEQ ID NO: 86
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 123 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuugaugagauacucauagugg

SEQ ID NO: 124 - RNA, 36 nt, Artificial Sequence; identical to SEQ ID NO: 88
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 125 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uacauugaugagauacucaugg

SEQ ID NO: 126 - RNA, 36 nt, Artificial Sequence; identical to SEQ ID NO: 90
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 127 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uucacauugaugagauacucgg

SEQ ID NO: 128 - RNA, 36 nt, Artificial Sequence; identical to SEQ ID NO: 92
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 129 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaucacauugaugagauacugg

SEQ ID NO: 130 - RNA, 36 nt, Artificial Sequence; identical to SEQ ID NO: 94
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 131 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaaauguacacaggcacaggg

SEQ ID NO: 132 - RNA, 36 nt, Artificial Sequence; identical to SEQ ID NO: 96
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 133 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuugaaguaaauguacacaggg

SEQ ID NO: 134 - RNA, 36 nt, Artificial Sequence; identical to SEQ ID NO: 98
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 135 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuguugaaguaaauguacacgg

SEQ ID NO: 136 - RNA, 36 nt, Artificial Sequence; identical to SEQ ID NO: 100
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 137 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uagaacaccauacauucugggg

SEQ ID NO: 138 - RNA, 36 nt, Artificial Sequence; identical to SEQ ID NO: 102
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 139 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
ugucauuuggaacucagcuggg

SEQ ID NO: 140 - RNA, 36 nt, Artificial Sequence; identical to SEQ ID NO: 104
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 141 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaaacagguggaaggucauugg

SEQ ID NO: 142 - RNA, 36 nt, Artificial Sequence; identical to SEQ ID NO: 106
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 143 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaauaaacagguggaaggucgg

SEQ ID NO: 144 - RNA, 36 nt, Artificial Sequence; identical to SEQ ID NO: 108
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 145 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uagcaaucaugaaggugagcgg

SEQ ID NO: 146 - RNA, 36 nt, Artificial Sequence; identical to SEQ ID NO: 110
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 147 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuggcagcaaucaugaaggugg

SEQ ID NO: 148 - RNA, 25 nt, Artificial Sequence
  synthetic construct
augaguaucucauuaauguaauuca

SEQ ID NO: 149 - RNA, 25 nt, Artificial Sequence
  synthetic construct
aguaucucauuaaugugauacaugc

SEQ ID NO: 150 - DNA, 25 bp, Artificial Sequence
  synthetic construct
  misc_feature (1)..(24): ribonucleotides
auuaaugugauucaugcuuaccagt

SEQ ID NO: 151 - RNA, 25 nt, Artificial Sequence
  synthetic construct
gagcauaguucuuuuugaaaacaag

SEQ ID NO: 152 - RNA, 25 nt, Artificial Sequence
  synthetic construct
agcauaguucuuuuugaaaacaaga

SEQ ID NO: 153 - RNA, 25 nt, Artificial Sequence
  synthetic construct
agaaagcaucacaaaaauaauugaa

SEQ ID NO: 154 - RNA, 25 nt, Artificial Sequence
  synthetic construct
gaaagcaucacaaaaauauaugaaa

SEQ ID NO: 155 - DNA, 25 bp, Artificial Sequence
  synthetic construct
  misc_feature (1)..(23): ribonucleotides
  misc_feature (25)..(25): ribonucleotides
caucacaaaaauauuugaaauugta

SEQ ID NO: 156 - DNA, 25 bp, Artificial Sequence
  synthetic construct
  misc_feature (1)..(23): ribonucleotides
acagaugauuuuacuugcuaauatt

SEQ ID NO: 157 - DNA, 25 bp, Artificial Sequence
  synthetic construct
  misc_feature (1)..(23): ribonucleotides
  misc_feature (25)..(25): ribonucleotides
cagaugauuuuacuugcuaauauta

SEQ ID NO: 158 - RNA, 25 nt, Artificial Sequence
  synthetic construct
auuuuacuugcuaauauuaacucag

SEQ ID NO: 159 - DNA, 25 bp, Artificial Sequence
  synthetic construct
  misc_feature (1)..(23): ribonucleotides
  misc_feature (25)..(25): ribonucleotides
aaguuacugucucuugguaaauata

SEQ ID NO: 160 - DNA, 25 bp, Artificial Sequence
  synthetic construct
  misc_feature (1)..(24): ribonucleotides
ggaaaaguuauuguagcugauucat

SEQ ID NO: 161 - DNA, 25 bp, Artificial Sequence
  synthetic construct
  misc_feature (1)..(23): ribonucleotides
gaaaaguuauuguagcuguaucatt

SEQ ID NO: 162 - DNA, 25 bp, Artificial Sequence
  synthetic construct
  misc_feature (1)..(24): ribonucleotides
aaaguuauuguagcuguuuaauugt

SEQ ID NO: 163 - DNA, 25 bp, Artificial Sequence
  synthetic construct
  misc_feature (1)..(23): ribonucleotides
  misc_feature (25)..(25): ribonucleotides
aaguuauuguagcuguuucauugta

SEQ ID NO: 164 - DNA, 25 bp, Artificial Sequence
  synthetic construct
  misc_feature (1)..(23): ribonucleotides
gaaggugaaauaaucuauaacuutt

SEQ ID NO: 165 - RNA, 25 nt, Artificial Sequence
  synthetic construct
guuuugguuuaauauaacaaauaac

SEQ ID NO: 166 - RNA, 25 nt, Artificial Sequence
  synthetic construct
gauagagaauuuugauuuuaacaac

SEQ ID NO: 167 - RNA, 25 nt, Artificial Sequence
  synthetic construct
auagagaauuuugauuuuaacaaca

SEQ ID NO: 168 - RNA, 25 nt, Artificial Sequence
  synthetic construct
agaauuuugauuuuaacaaaauaaa

SEQ ID NO: 169 - RNA, 25 nt, Artificial Sequence
  synthetic construct
agugaauuguucuauuugaacucaa

SEQ ID NO: 170 - DNA, 25 bp, Artificial Sequence
  synthetic construct
gugaauuguucuauuugacaucaat

SEQ ID NO: 171 - RNA, 25 nt, Artificial Sequence
  synthetic construct
acagaaaagcuaauugagaccuauu

SEQ ID NO: 172 - RNA, 25 nt, Artificial Sequence
  synthetic construct
cagaaaagcuaauugagaccuauuu

SEQ ID NO: 173 - RNA, 25 nt, Artificial Sequence
  synthetic construct
gcuaauugagaccuauuucuccaaa

SEQ ID NO: 174 - RNA, 25 nt, Artificial Sequence
  synthetic construct
cuaauugagaccuauuucuccaaaa

SEQ ID NO: 175 - RNA, 25 nt, Artificial Sequence
  synthetic construct
gacuaugaguaucucaucaauguga

SEQ ID NO: 176 - RNA, 25 nt, Artificial Sequence
  synthetic construct
acuaugaguaucucaucaaugugau

SEQ ID NO: 177 - RNA, 25 nt, Artificial Sequence
  synthetic construct
augaguaucucaucaaugugaucca

SEQ ID NO: 178 - RNA, 25 nt, Artificial Sequence
  synthetic construct
gaguaucucaucaaugugauccaug

SEQ ID NO: 179 - RNA, 25 nt, Artificial Sequence
  synthetic construct
aguaucucaucaaugugauccaugc

SEQ ID NO: 180 - RNA, 25 nt, Artificial Sequence
  synthetic construct
cugugccuguguacauuuacuucaa

SEQ ID NO: 181 - RNA, 25 nt, Artificial Sequence
  synthetic construct
cuguguacauuuacuucaacaccug

SEQ ID NO: 182 - RNA, 25 nt, Artificial Sequence
  synthetic construct
guguacauuuacuucaacaccugga

SEQ ID NO: 183 - RNA, 25 nt, Artificial Sequence
  synthetic construct
ccagaauguaugguguucucccaug

SEQ ID NO: 184 - RNA, 25 nt, Artificial Sequence
  synthetic construct
cagcugaguuccaaaugaccuucca

SEQ ID NO: 185 - RNA, 25 nt, Artificial Sequence
  synthetic construct
aaugaccuuccaccuguuuauugcu

SEQ ID NO: 186 - RNA, 25 nt, Artificial Sequence
  synthetic construct
gaccuuccaccuguuuauugcugca

SEQ ID NO: 187 - RNA, 25 nt, Artificial Sequence
  synthetic construct
gcucaccuucaugauugcugccacu

SEQ ID NO: 188 - RNA, 25 nt, Artificial Sequence
  synthetic construct
accuucaugauugcugccacuuaca

SEQ ID NO: 189 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 190 - RNA, 16 nt, Artificial Sequence
  synthetic construct
gcagccgaaaggcugc

SEQ ID NO: 191 - RNA, 36 nt, Artificial Sequence; identical to SEQ ID NO: 76
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 192 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uucucaauuagcuuuucugugg

SEQ ID NO: 193 - RNA, 36 nt, Artificial Sequence; identical to SEQ ID NO: 16
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 194 - RNA, 36 nt, Artificial Sequence; identical to SEQ ID NO: 18
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 195 - RNA, 36 nt, Artificial Sequence; identical to SEQ ID NO: 22
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 196 - RNA, 36 nt, Artificial Sequence; identical to SEQ ID NO: 22
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 197 - RNA, 36 nt, Artificial Sequence
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified
cgggugugucauuguuuggagcagccgaaaggcugc

SEQ ID NO: 198 - RNA, 36 nt, Artificial Sequence
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified
gaaaagcuaauugagaccuagcagccgaaaggcugc

SEQ ID NO: 199 - RNA, 36 nt, Artificial Sequence; identical to SEQ ID NO: 18
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′-O-methyl modified
  modified_base (4)..(4): 2′-O-methyl modified
  modified_base (5)..(5): 2′-O-methyl modified
  modified_base (6)..(6): 2′-O-methyl modified
  modified_base (7)..(7): 2′-O-methyl modified
  modified_base (8)..(8): 2′-fluoro modified
  modified_base (9)..(9): 2′-fluoro modified
  modified_base (10)..(10): 2′-fluoro modified
  modified_base (11)..(11): 2′-fluoro modified
  modified_base (12)..(12): 2′-O-methyl modified
  modified_base (13)..(13): 2′-O-methyl modified
  modified_base (14)..(14): 2′-O-methyl modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): 2′-O-methyl modified
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 200 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uagcaaguaaaaucaucugugg

SEQ ID NO: 201 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuagcaaguaaaaucaucuggg

SEQ ID NO: 202 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaccaagagacaguaacuugg

SEQ ID NO: 203 - RNA, 22 nt, Artificial Sequence
  synthetic construct
  misc_feature (1)..(4): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuaccaagagacaguaacuugg

SEQ ID NO: 204 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uccaaacaaugacacacccggg

SEQ ID NO: 205 - RNA, 22 nt, Artificial Sequence
  synthetic construct
  misc_feature (1)..(3): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaggucucaauuagcuuuucgg

SEQ ID NO: 206 - RNA, 22 nt, Artificial Sequence
  synthetic construct
  misc_feature (1)..(4): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uuagcaaguaaaaucaucuggg

SEQ ID NO: 207 - RNA, 22 nt, Artificial Sequence
  synthetic construct
  misc_feature (1)..(4): linked via phosphorothioate
  modified_base (1)..(1): 4′-monomoethylphosphonate-2′-O-methyl modified
  modified_base (1)..(1): 2′-O-methyl modified
  modified_base (2)..(2): 2′-fluoro modified
  modified_base (3)..(3): 2′-fluoro modified
  modified_base (4)..(4): 2′-fluoro modified
  modified_base (5)..(5): 2′-fluoro modified
  modified_base (6)..(6): 2′-O-methyl modified
  modified_base (7)..(7): 2′-fluoro modified
  modified_base (8)..(8): 2′-O-methyl modified
  modified_base (9)..(9): 2′-O-methyl modified
  modified_base (10)..(10): 2′-fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′-fluoro modified
  modified_base (15)..(15): 2′-O-methyl modified
  modified_base (16)..(16): 2′-O-methyl modified
  modified_base (17)..(17): 2′-O-methyl modified
  modified_base (18)..(18): 2′-O-methyl modified
  modified_base (19)..(19): 2′-O-methyl modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uucucaauuagcuuuucugugg

SEQ ID NO: 208 - RNA, 36 nt, Artificial Sequence; identical to SEQ ID NO: 197
  synthetic construct

SEQ ID NO: 209 - RNA, 36 nt, Artificial Sequence; identical to SEQ ID NO: 198
  synthetic construct

SEQ ID NO: 210 - RNA, 22 nt, Artificial Sequence
  synthetic construct
uccaaacaaugacacacccggg

SEQ ID NO: 211 - RNA, 22 nt, Artificial Sequence
  synthetic construct
uaggucucaauuagcuuuucgg

SEQ ID NO: 212 - RNA, 19 nt, Artificial Sequence
  synthetic construct
acagaaaagcuaauugaga

SEQ ID NO: 213 - RNA, 19 nt, Artificial Sequence
  synthetic construct
cagaaaagcuaauugagac

SEQ ID NO: 214 - RNA, 19 nt, Artificial Sequence
  synthetic construct
gaaaagcuaauugagaccu

SEQ ID NO: 215 - RNA, 19 nt, Artificial Sequence
  synthetic construct
gcuaauugagaccuauuuc

SEQ ID NO: 216 - RNA, 19 nt, Artificial Sequence
  synthetic construct
cuaauugagaccuauuucu

SEQ ID NO: 217 - RNA, 19 nt, Artificial Sequence
  synthetic construct
gacuaugaguaucucauca

SEQ ID NO: 218 - RNA, 19 nt, Artificial Sequence
  synthetic construct
acuaugaguaucucaucaa

SEQ ID NO: 219 - RNA, 19 nt, Artificial Sequence
  synthetic construct
augaguaucucaucaaugu

SEQ ID NO: 220 - RNA, 19 nt, Artificial Sequence
  synthetic construct
gaguaucucaucaauguga

SEQ ID NO: 221 - RNA, 19 nt, Artificial Sequence
  synthetic construct
aguaucucaucaaugugau

SEQ ID NO: 222 - RNA, 19 nt, Artificial Sequence
  synthetic construct
cgggugugucauuguuugg

SEQ ID NO: 223 - RNA, 19 nt, Artificial Sequence
  synthetic construct
cugugccuguguacauuua

SEQ ID NO: 224 - RNA, 19 nt, Artificial Sequence
  synthetic construct
cuguguacauuuacuucaa

SEQ ID NO: 225 - RNA, 19 nt, Artificial Sequence
  synthetic construct
guguacauuuacuucaaca

SEQ ID NO: 226 - RNA, 19 nt, Artificial Sequence
  synthetic construct
ccagaauguaugguguucu

SEQ ID NO: 227 - RNA, 19 nt, Artificial Sequence
  synthetic construct
cagcugaguuccaaaugac

SEQ ID NO: 228 - RNA, 19 nt, Artificial Sequence
  synthetic construct
aaugaccuuccaccuguuu

SEQ ID NO: 229 - RNA, 19 nt, Artificial Sequence
  synthetic construct
gaccuuccaccuguuuauu

SEQ ID NO: 230 - RNA, 19 nt, Artificial Sequence
  synthetic construct
gcucaccuucaugauugcu

SEQ ID NO: 231 - RNA, 19 nt, Artificial Sequence
  synthetic construct
accuucaugauugcugcca

SEQ ID NO: 232 - RNA, 19 nt, Artificial Sequence
  synthetic construct
acagaugauuuuacuugcu

SEQ ID NO: 233 - RNA, 19 nt, Artificial Sequence
  synthetic construct
cagaugauuuuacuugcua

SEQ ID NO: 234 - RNA, 19 nt, Artificial Sequence
  synthetic construct
aaguuacugucucuuggua

SEQ ID NO: 235 - RNA, 19 nt, Artificial Sequence
  synthetic construct
ucucaauuagcuuuucugu

SEQ ID NO: 236 - RNA, 19 nt, Artificial Sequence
  synthetic construct
gucucaauuagcuuuucug

SEQ ID NO: 237 - RNA, 19 nt, Artificial Sequence
  synthetic construct
aggucucaauuagcuuuuc

SEQ ID NO: 238 - RNA, 19 nt, Artificial Sequence
  synthetic construct
gaaauaggucucaauuagc

SEQ ID NO: 239 - RNA, 19 nt, Artificial Sequence
  synthetic construct
agaaauaggucucaauuag

SEQ ID NO: 240 - RNA, 19 nt, Artificial Sequence
  synthetic construct
ugaugagauacucauaguc

SEQ ID NO: 241 - RNA, 19 nt, Artificial Sequence
  synthetic construct
uugaugagauacucauagu

SEQ ID NO: 242 - RNA, 19 nt, Artificial Sequence
  synthetic construct
acauugaugagauacucau

SEQ ID NO: 243 - RNA, 19 nt, Artificial Sequence
  synthetic construct
ucacauugaugagauacuc

SEQ ID NO: 244 - RNA, 19 nt, Artificial Sequence
  synthetic construct
aucacauugaugagauacu

SEQ ID NO: 245 - RNA, 19 nt, Artificial Sequence
  synthetic construct
ccaaacaaugacacacccg

SEQ ID NO: 246 - RNA, 19 nt, Artificial Sequence
  synthetic construct
uaaauguacacaggcacag

SEQ ID NO: 247 - RNA, 19 nt, Artificial Sequence
  synthetic construct
uugaaguaaauguacacag

SEQ ID NO: 248 - RNA, 19 nt, Artificial Sequence
  synthetic construct
uguugaaguaaauguacac

SEQ ID NO: 249 - RNA, 19 nt, Artificial Sequence
  synthetic construct
agaacaccauacauucugg

SEQ ID NO: 250 - RNA, 19 nt, Artificial Sequence
  synthetic construct
gucauuuggaacucagcug

SEQ ID NO: 251 - RNA, 19 nt, Artificial Sequence
  synthetic construct
aaacagguggaaggucauu

SEQ ID NO: 252 - RNA, 19 nt, Artificial Sequence
  synthetic construct
aauaaacagguggaagguc

SEQ ID NO: 253 - RNA, 19 nt, Artificial Sequence
  synthetic construct
agcaaucaugaaggugagc

SEQ ID NO: 254 - RNA, 19 nt, Artificial Sequence
  synthetic construct
uggcagcaaucaugaaggu

SEQ ID NO: 255 - RNA, 19 nt, Artificial Sequence
  synthetic construct
agcaaguaaaaucaucugu

SEQ ID NO: 256 - RNA, 19 nt, Artificial Sequence
  synthetic construct
uagcaaguaaaaucaucug

SEQ ID NO: 257 - RNA, 19 nt, Artificial Sequence
  synthetic construct
uaccaagagacaguaacuu